SEQUENCE LISTING (47 sequences)

SEQ ID NO: 1 - protein, 8 aa, Artificial Sequence
  Synthetic Construct
IAAAEEEE

SEQ ID NO: 2 - protein, 8 aa, Artificial Sequence
  Synthetic Construct
FAAAEEEE

SEQ ID NO: 3 - protein, 8 aa, Artificial Sequence
  Synthetic Construct
VAAAEEEE

SEQ ID NO: 4 - protein, 8 aa, Artificial Sequence
  Synthetic Construct
YAAAEEEE

SEQ ID NO: 5 - protein, 9 aa, Artificial Sequence
  Synthetic Construct
IAAAEEEEK

SEQ ID NO: 6 - protein, 9 aa, Artificial Sequence
  Synthetic Construct
VAAAEEEEK

SEQ ID NO: 7 - protein, 9 aa, Artificial Sequence
  Synthetic Construct
YAAAEEEEK

SEQ ID NO: 8 - protein, 9 aa, Artificial Sequence
  Synthetic Construct
AYAAEEEEK

SEQ ID NO: 9 - protein, 9 aa, Artificial Sequence
  Synthetic Construct
AAYAEEEEK

SEQ ID NO: 10 - protein, 9 aa, Artificial Sequence
  Synthetic Construct
AAAYEEEEK

SEQ ID NO: 11 - protein, 8 aa, Artificial Sequence
  Synthetic Construct
YAAAEEEK

SEQ ID NO: 12 - protein, 5 aa, Artificial Sequence
  Synthetic Construct
IAAAK

SEQ ID NO: 13 - protein, 7 aa, Artificial Sequence
  Synthetic Construct
IAAAEEK

SEQ ID NO: 14 - protein, 6 aa, Artificial Sequence
  Synthetic Construct
IEEEEK

SEQ ID NO: 15 - protein, 7 aa, Artificial Sequence
  Synthetic Construct
IAEEEEK

SEQ ID NO: 16 - protein, 8 aa, Artificial Sequence
  Synthetic Construct
IAAAEEEK

SEQ ID NO: 17 - protein, 8 aa, Artificial Sequence
  Synthetic Construct
AYAAEEEK

SEQ ID NO: 18 - protein, 8 aa, Artificial Sequence
  Synthetic Construct
AAYAEEEK

SEQ ID NO: 19 - protein, 8 aa, Artificial Sequence
  Synthetic Construct
AAAYEEEK

SEQ ID NO: 20 - protein, 5 aa, Artificial Sequence
  Synthetic Construct
IAAAK

SEQ ID NO: 21 - protein, 7 aa, Artificial Sequence
  Synthetic Construct
IAAAEEK

SEQ ID NO: 22 - protein, 5 aa, Artificial Sequence
  Synthetic Construct
IEEEK

SEQ ID NO: 23 - protein, 6 aa, Artificial Sequence
  Synthetic Construct
IAEEEK

SEQ ID NO: 24 - protein, 7 aa, Artificial Sequence
  Synthetic Construct
IAAAEEK

SEQ ID NO: 25 - protein, 10 aa, Artificial Sequence
  Synthetic Construct
VVAAEEEEEK

SEQ ID NO: 26 - protein, 10 aa, Artificial Sequence
  Synthetic Construct
YYAAEEEEEK

SEQ ID NO: 27 - protein, 11 aa, Artificial Sequence
  Synthetic Construct
YYAAEEEEEEK

SEQ ID NO: 28 - protein, 12 aa, Artificial Sequence
  Synthetic Construct
YYAAEEEEEEEK

SEQ ID NO: 29 - protein, 10 aa, Artificial Sequence
  Synthetic Construct
YYAAAEEEEK

SEQ ID NO: 30 - protein, 8 aa, Artificial Sequence
  Synthetic Construct
VVAAEEEE

SEQ ID NO: 31 - protein, 8 aa, Artificial Sequence
  Synthetic Construct
AAVVEEEE

SEQ ID NO: 32 - protein, 8 aa, Artificial Sequence
  Synthetic Construct
AAYYEEEE

SEQ ID NO: 33 - protein, 8 aa, Artificial Sequence
  Synthetic Construct
AAIIEEEE

SEQ ID NO: 34 - protein, 8 aa, Artificial Sequence
  Synthetic Construct
YYYYEEEE

SEQ ID NO: 35 - protein, 10 aa, Artificial Sequence
  Synthetic Construct
IAAAEEEEYK

SEQ ID NO: 36 - protein, 7 aa, Artificial Sequence
  Synthetic Construct
TTTEEEE

SEQ ID NO: 37 - protein, 8 aa, Artificial Sequence
  Synthetic Construct
AATTEEEE

SEQ ID NO: 38 - protein, 5 aa, Artificial Sequence
  Synthetic Construct
IAAAE

SEQ ID NO: 39 - protein, 5 aa, Artificial Sequence
  Synthetic Construct
MAAAE

SEQ ID NO: 40 - protein, 9 aa, Artificial Sequence
  Synthetic Construct
MAAAEEEEK

SEQ ID NO: 41 - protein, 8 aa, Artificial Sequence
  Synthetic Construct
AIAAEEEE

SEQ ID NO: 42 - protein, 8 aa, Artificial Sequence
  Synthetic Construct
AAIAEEEE

SEQ ID NO: 43 - protein, 8 aa, Artificial Sequence
  Synthetic Construct
AAAIEEEE

SEQ ID NO: 44 - protein, 9 aa, Artificial Sequence
  Synthetic Construct
AIAAEEEEK

SEQ ID NO: 45 - protein, 9 aa, Artificial Sequence
  Synthetic Construct
VVVAAAEEE

SEQ ID NO: 46 - protein, 9 aa, Artificial Sequence
  Synthetic Construct
YYAAEEEEK

SEQ ID NO: 47 - protein, 10 aa, Artificial Sequence
  Synthetic Construct
YAAAEEEEEK

(19) United States
(12) Patent Application Publication
(10) Pub. No.: US 2017/0224833 A1
(43) Pub. Date: Aug. 10, 2017

(54) SELF-ASSEMBLING MOLECULES THAT ACCUMULATE IN ACIDIC TUMOR MICROENVIRONMENTS

(71) Applicant: OHIO STATE INNOVATION FOUNDATION, Columbus, OH (US)

(72) Inventors: Joshua E. Goldberger, Columbus, OH (US); Michael F. Tweedle, Columbus, OH (US)

(21) Appl. No.: 15/502,363

(22) PCT Filed: Aug. 7, 2015
(86) PCT No.: PCT/US15/44346

§ 371 Date: Feb. 7, 2017

Related U.S. Application Data
(60) Provisional application No. 62/034,681, filed on Aug. 7, 2014.

(51) Int. Cl. A61B 5/055; A61K 49/00; A61K 9/107; A61K 49/14; A61K 49/18

(52) U.S. Cl. CPC A61K 47/48246; A61K 49/14; A61K 49/0056; A61K 49/0082; A61K 49/1809; A61K 9/1075

(57) ABSTRACT

Disclosed are compositions that contain a plurality of biocompatible self-assembling molecules that transform from isolated molecules or spherical micelles while in blood serum into cylindrical nanofibers in the acidic extracellular environment of tumors, which can be used to achieve a higher relative concentration of imaging drug delivery, or radiotherapeutic agents at the tumor site compared to non-tumor tissues. This transition is rapid and reversible, indicating the system is in thermodynamic equilibrium.

### CROSS-REFERENCE TO RELATED APPLICATIONS

This application claims benefit of U.S. Provisional Application No. 62/034,681, filed Aug. 7, 2014, which is hereby incorporated herein by reference in its entirety.

### BACKGROUND

Early detection and identification of a suspected tumor in a localized stage significantly improves the chances for successful treatment and elimination of the cancerous tissue. A large number of imaging strategies have therefore been designed, using a variety of techniques and modalities to aid the physician in making an accurate diagnosis as early as possible. Unfortunately, conventional imaging techniques such as computerized tomography (CT) and magnetic resonance imaging (MRI) are limited in their ability to afford a conclusive diagnosis of a suspected lesion, since they are only capable of observing differences in the density or morphology of tissues. A more invasive and costly biopsy procedure is often necessary to provide a definitive diagnosis. In contrast, nuclear medicine techniques such as positron emission tomography (PET) and single photon emission tomography (SPECT) can provide functional or biochemical information about a particular organ or area of interest. However, the success of these nuclear imaging techniques depends in large part on the selective uptake and detection of appropriate radiopharmaceuticals and on the spatial resolution of the imaging technique. Spatial resolution limits the sensitivity of SPECT and PET to lesions >1 cm in diameter. Selective uptake, in turn, depends upon the development of radiopharmaceuticals with a high degree of specificity for the target tissue. Tumor-localizing agents developed thus far for oncological use have had only limited application. In PET imaging, fluorodeoxyglucose (¹⁸F-FDG) is used for imaging tumors in oncology, where a static FDG PET scan is performed and the tumor FDG uptake is analyzed in terms of Standardized Uptake Value (SUV). ¹⁸F-FDG has had the widest application, despite some limitations in specificity and the aforementioned limitation in sensitivity. There is therefore a need in the art for imaging and therapeutic agents that accumulate in tumor tissue but exhibit a rapid clearance from non-target tissues, and also a need for such agents that can be imaged by higher spatial resolution imaging scanners like MRI and CT. Such agents could assist in the non-invasive imaging of primary tumors and metastases and could serve as carriers for cytotoxic agents for site-specific eradication of malignant tumor tissue or acidic inflamed tissue.

### SUMMARY

Disclosed are compositions that transform into larger, bulky, more slowly diffusing materials upon reaching an acidic extracellular tissue environment, which will cause a higher relative concentration in the acidic environment for imaging, non radioactive drug delivery, or radiotherapeutic agents at the tissue site compared to the surrounding tissue or circulation. In particular, self-assembling molecules are disclosed that transform from isolated molecules or spherical micelles while in blood serum into cylindrical nanofibers in an acidic extracellular microenvironment (e.g., malignant tumor tissue or inflamed joints). This transition is rapid and reversible, indicating the system is in thermodynamic equilibrium. A composition is therefore disclosed that contains a plurality of biocompatible self-assembling molecules that are present as isolated molecules or spherical micelles in the neutral pH and isotonic conditions of blood serum and normal extracellular environment, and that transform into cylindrical nanofibers in an acidic extracellular environment. At least a portion of the plurality of biocompatible self-assembling molecules may be conjugated to a diagnostic or therapeutic agent such that self assembly of the molecules in the acidic environment of a tissue results in accumulation of the diagnostic or therapeutic agent in the tissue.

### DESCRIPTION OF DRAWINGS

FIG. 1 is a schematic of target reversible, pH-triggered morphological transition of self-assembling molecules from single molecules or spherical micelles (neutral or basic pH) to cylindrical nanofibers (acidic pH) in a physiological solution.

FIGS. 2A and 2B are graphs showing the circular dichroism (CD) spectra of PA1 molecules to characterize the morphology of the molecule at various pH values. FIG. 2B shows the CD spectra of the same PA1 molecules at alternating pH to show the reversibility of the pH-triggered morphology transition. FIG. 2C is a graph showing critical aggregation concentration (CAC) of PA1 at pH 6.6 using the pyrene 1:3 method. All CD and CAC samples were prepared in 150 mM NaC, and 2.2 mM CaCl₂.

FIGS. 3A and 3B are transmission electron microscopy (TEM) images of 0.5 mM of PA1, measured at pH 6.0 (FIG. 3A) and pH 10.0 (FIG. 3B). FIG. 3C is a graph showing concentration-pH self-assembly phase diagram of PA1 as determined via CAC (diamonds), and CD (squares) measurements. All samples were prepared in 150 mM NaCl, and 2.2 mM CaCl₂. The white area corresponds to a region where the self-assembled morphology is uncertain due to the lack of suitable experimental techniques.

FIGS. 4A and 4B are TEM images of 0.5 mM of PA5, measured at pH 4.0 (FIG. 4A), and pH 10.0 (FIG. 4B). FIG. 4C is a graph showing concentration-pH self-assembly phase diagram of PA5 as determined via CAC (diamonds), and CD (square) measurements. All samples were prepared in 150 mM NaCl, and 2.2 mM CaCl₂.

FIG. 5 is a synthesis scheme for a protected tri-tert-butyl ester 1-substituted 1,4,7,-tricarboxymethyl 1,4,7,10 tetraazacyclododecane triacetic acid (DO3A) derivative.

FIGS. 6A and 6B are TEM images of 10 μM PA1 at pH 6 (FIG. 6A) and pH 8 (FIG. 6B) from samples that were dropcast three minutes after pH adjustment. Solutions were prepared in 150 mM NaCl, 2.2 mM CaCl₂. No fibers and only staining artifacts were observed across the TEM grid at pH of 8.

FIG. 7 is a graph showing CAC of PA1 at pH 6.0 (triangle) and pH 7.8 (square) using the pyrene 1:3 method.

FIGS. 8A and 8B are graphs showing the CD spectra of 30 μM PA1 (FIG. 8A) or 15 μM PA1 (FIG. 8B) at different basic pH values. All samples were prepared in 150 mM NaCl, and 2.2 mM CaCl₂.

FIGS. 9A to 9C are graphs showing the CD spectra of 10 μM PA2 (FIG. 9A), PA3 (FIG. 9B), or PA4 (FIG. 9C) at different pH values. All samples were prepared at in 150 mM NaCl, and 2.2 mM CaCl₂.

FIG. 10 is a graph showing the CD spectra of 10 μM PA3 at alternating pH to show the reversibility of the pH-triggered morphology transition. All samples were prepared at in 150 mM NaCl, and 2.2 mM CaCl₂.

FIGS. 11A to 11D are pH titration curves of 10 μM PA1 (FIG. 11A), PA2 (FIG. 11B), PA3 (FIG. 11C), and PA4 (FIG. 11D) in 150 mM NaCl, 2.2 mM CaCl₂ against NaOH.

FIG. 12 is a graph showing the CD spectra of 10 μM PA5 at different pH values. All samples were prepared at in 150 mM NaCl, and 2.2 mM CaCl₂.

FIGS. 13A and 13B are graphs showing the CD spectra of 20 μM PA5 (FIG. 13A) or 500 μM PA5 (FIG. 13B) at different pH values. All samples were prepared at in 150 mM NaCl, and 2.2 mM CaCl₂.

FIGS. 14A and 14B are graphs showing the CD spectra of 10 μM PA6 (FIG. 14A) or 500 μM PA6 (FIG. 14B) at different pH values. All samples were prepared at in 150 mM NaCl, and 2.2 mM CaCl₂.

FIG. 15 is a graph showing CAC of PA5 at pH 6.0 (triangle) and pH 7.6 (square) using the pyrene 1:3 method.

FIG. 16 is a concentration-pH phase diagram of pure PA5 (light grey) and the PAmix1 (dark grey) as determined via CD (solid diamonds) and CAC (hollow diamonds) measurements, respectively. All measurements were done in 150 mM NaCl and 2.2 mM CaCl₂.

FIG. 17A is a graph showing pH dependent fluorescence anisotropy (FA) of 100 μM PAmix1. The inset shows the fluorescence emission from the Ru(bipy)₃ label in PA66 in the mixture. FIG. 17B shows pH dependent CD spectra of 100 μM PAmix1.

FIG. 18A shows fluorescence emission from 100 μM PAmix1 in pure serum along with serum auto-fluorescence background. FIG. 18B shows pH dependent FA of 100 μM PAmix1 and PAmix2 in salts and pure serum. FIG. 18C shows pH reversibility of morphology transition in 100 μM PAmix2 in serum. FIG. 18D shows time-dependent stability of spherical micelles and nanofibers in 100 μM PAmix2 in serum via FA measurements.

FIG. 19A shows pH dependent FA of 100 μM PAmix1 in 0-4% v/v diluted serum solutions. FIG. 19B shows kinetics of morphology switch of 100 μM PAmix1 in 1.5% serum via time-dependent FA measurements. FIGS. 19C and 19D show TEM images of 100 μM PAmix1 in 1.5% serum at pH 6.85 (FIG. 19C) and pH 9.21 (FIG. 19D).

FIG. 20 is a graph showing pH dependent FA of 100 μM PAmix1 in salts, 1.5% serum, 7.8 μM MSA and 26 μM PEG.

FIG. 21 shows concentration dependent CD transition pH values of PAmix2 in artificial and real serum overlaid on the phase diagram of 100 μM PA65 in artificial serum.

FIGS. 22A-22D are reverse-phase HPLC traces of PA5 (FIG. 22A), PA 66 (FIG. 22B), PA65 (FIG. 22C), and PA67 (FIG. 22D). The elution gradient changes linearly from 10% v/v MeCN in water (containing 0.1%, v/v NH₄OH) at 0 min to 100% MeCN (containing 0.1% v/v NH₄OH) over a period of 60 minutes.

FIGS. 23A-23D are ESI-mass spectra of PA5 (FIG. 23A, M.W. 1767 g·mol), PA 66 (FIG. 23B, M.W. 1835 g·mol), PA65 (FIG. 23C, M.W. 1784 g·mol), and PA67 (FIG. 23D, M.W. 1854 g·mol).

FIG. 24A is a plot showing pH dependent CAC of PAmix1 using the pyrene 1:3 method in 150 mM NaCl, 2.2 mM CaCl₂ at pH 5 and pH 10. FIGS. 24B and 24C are pyrene fluorescence spectra for different PAmix1 concentrations at pH 5 (FIG. 24B) and pH 10 (FIG. 24C).

FIGS. 25A-25C are CD spectra of 10 μM (FIG. 25A), 50 μM (FIG. 25B), and 50 μM (FIG. 25C) PAmix1 at different pH values in 150 mM NaCl, 2.2 mM CaCl₂.

FIG. 26 is a CD spectrum showing reversibility of pH-triggered morphology transition in 100 μM PAmix1 in 150 mM NaCl, 2.2 mM CaCl₂. The CD traces were collected within 2-3 min of pH adjustment.

FIG. 27 shows PA66 concentration dependent fluorescence of 100 μM PAmix1 in pure mouse blood serum.

FIG. 28 shows pH dependent fluorescence of 100 μM PAmix1 in 150 mM NaCl, 2.2 mM CaCl₂.

FIG. 29 shows time and pH dependent FA of MSA-dye conjugate control.

FIG. 30 is a TEM image of 1.5% v/v serum in 150 mM NaCl, 2.2 mM CaCl₂ at pH 6.0.

FIG. 31 shows pH dependent FA of 100 μM PAmix1 in 1.8 mM PEG. 150 mM NaCl and 2.2 mM CaCl₂.

FIG. 32A shows CD transition points of 100 μM PAmix1 in 3.0 (grey) and 4.0 (black) mM CaCl₂, 150 mM NaCl overlaid on the phase diagram of PA5 in 2.2 mM CaCl₂, 150 mM NaCl. FIGS. 32B and 32C show pH dependent CD spectra for the 3.0 mM CaCl₂ (FIG. 32B) and 4.0 mM CaCl₂ (FIG. 32C).

FIGS. 33A-33B show CD spectra of 10 μM (FIG. 33A) and 500 μM (FIG. 33B) PA65 in 1.8 mM PEG, 150 mM NaCl and 2.2 mM CaCl₂.

FIG. 34 shows pH dependent CAC of PA65 in 1.8 mM PEG, 150 mM NaCl and 2.2 mM CaCl₂ (CACs at pH 5.5 and pH 10.0.

FIG. 35 shows concentration dependent FA of 20 μM, 50 μM, and 100 μM PAmix2 in pure serum.

FIGS. 36A-36B show concentration dependent CD spectra of 20 μM (FIG. 36A) and 100 μM (FIG. 36B) PAmix2 in 1.8 mM PEG. 150 mM NaCl and 2.2 mM CaCl₂.

FIG. 37 shows CD spectrum of 1.5% PA66 only in 150 mM NaCl, 2.2 mM CaCl₂.

FIGS. 38A and 38B are TEM images of 100 μM PA68 at pH 5.0 (FIG. 38A) and pH 8.3 (FIG. 38B). FIG. 38C shows concentration-pH phase diagram of PA68 (black) overlaid on the same for PA1 (gray), as determined via CD (squares) and CAC (triangles) measurements. For both PAs, the top area corresponds to nanofiber morphologies, and the bottom area corresponds to unassembled single molecules. The self-assembled morphology in the region of the phase diagram between these two regions is uncertain due to the lack of experimental techniques in this concentration range. All measurements were performed in 150 mM NaCl and 2.2 mM CaCl₂.

FIGS. 39A and 39B are TEM images of PA71 at 150 M, pH 6.6 (FIG. 39A), and 1 mM, pH 10.0 (FIG. 39B). FIG. 39C shows concentration-pH phase diagram of PA 71 (black) overlaid on the same for PA5 (gray), as determined via CD (squares) and CAC (triangles) measurements. All measurements were performed in 150 mM NaCl and 2.2 mM CaCl₂.

FIGS. 40A and 40B are TEM images of 100 μM PA69 at pH 8.0 (FIG. 40A) and PA70 at pH 8.0 (FIG. 40B). pH-dependent CD spectra of 5 μM PA69 (FIG. 40C) and 10 μM PA70 (FIG. 40D). All measurements were conducted in 150 mM NaCl and 2.2 mM CaCl₂.

FIGS. 41A to 41D are reverse phase HPLC chromatograms of synthesized PA68 (FIG. 41A), PA69 (FIG. 41B), PA70 (FIG. 41C), and PA71 (FIG. 41D).

FIGS. 42A to 42D are electro-spray Ionization Mass spectra of synthesized PA68 (FIG. 42A), PA69 (FIG. 42B), PA70 (FIG. 42C), and PA71 (FIG. 42D).

FIGS. 43A and 43B are pH dependent CAC for PA68 (FIG. 43A) and PA71 (FIG. 43B) using the pyrene 1:3 method (two pH points shown for clarity) in 150 mM NaCl and 2.2 mM CaCl₂.

FIGS. 44A to 44D are pH-dependent Circular Dichroism spectra of PA68 at 2.8 μM (FIG. 44A), 5.6 μM (FIG. 44B), 8.4 μM (FIG. 44C), 16.8 μM (FIG. 44D) in 150 mM NaCl and 2.2 mM CaCl₂.

FIGS. 45A to 45F are pH-dependent Circular Dichroism spectra of PA71 at 4.5 μM (FIG. 45A), 8.9 μM (FIG. 45B), 22.3 μM (FIG. 45C), 45 μM (FIG. 45D), 134 μM (FIG. 45E), 0.89 mM (FIG. 45F) in 150 mM NaCl and 2.2 mM CaCl₂.

FIGS. 46A and 46B are pH-dependent Circular Dichroism spectra of PA69 at 6.3 μM (FIG. 46A) and 19 μM (FIG. 46B).

FIG. 47 is a pH-dependent Circular Dichroism spectra of 30 μM PA70.

FIGS. 48A and 48B are TEM images of 500 μM PA1 at pH 10 (FIG. 48A) and pH 5 (FIG. 48B). FIG. 48C is a pH-dependent Circular Dichroism spectra of 10 μM PA73 at pH values ranging from 5-11 in 150 mM NaCl, 2.2 mM CaCl₂.

FIG. 49A is a pH-dependent Circular Dichroism spectra of 10 μM PA74. FIG. 49B shows the ratio of pyrene fluorescence emission at 376 nm and 392 nm at various concentrations of PA74 in order to determine the critical aggregation concentrations (dashed line) of PA74 at pH of 7 (squares) and pH of 9 (triangles). All samples measured in 150 mM NaCl, 2.2 mM CaCl₂.

FIG. 50A is a concentration-pH phase diagram of PA74 and PA75 in 150 mM NaCl and 2.2 mM CaCl₂. FIGS. 50B and 50C are TEM images of PA74 at 500 μM at pH 5 depicting nanofiber morphology (FIG. 50B) and 500 μM at pH 9 depicting spherical micelle morphology (FIG. 50C). FIGS. 50D and 50E are TEM images of PA75 at 100 μM at pH 5 depicting nanofiber morphology (FIG. 50D) and 100 μM at pH 9 depicting spherical micelle morphology (FIG. 50E).

FIG. 51 is a pH-dependent Circular Dichroism spectra of a mixture of 10% PA79 and 90% PA5, dissolved in artificial mouse serum (150 mM NaCl, 2.2 mM CaCl₂, and 1.8 mM of 20 kDa PEG salt solution). The transition pH occurs between pH 7.04 and 8.31.

FIG. 52 is a pH-dependent Circular Dichroism spectra of a 100 M solution of mixed PA that is 13% PA79 and 87% PA5, dissolved in artificial mouse serum. The transition pH occurs between pH 7.11 and 6.82.

FIG. 53 is a pH-dependent Circular Dichroism spectra of a mixture of 15% PA79 and 85% PA5, dissolved in artificial mouse serum. The transition pH occurs between pH 6.85 and 6.47.

FIG. 54 is a pH-dependent Circular Dichroism spectra of a 100 jiM solution of mixed PA that is 5% PA79 and PA6, dissolved in artificial mouse serum. The transition pH occurs between pH 6.75 and 7.31.

### DETAILED DESCRIPTION

Disclosed is a composition that, upon reaching the acidic extracellular tumor environment, transforms into a bulky, more slowly diffusing material, which can be used to achieve a higher relative concentration of diagnostic or therapeutic drugs for imaging, non-radioactive drug delivery, or radiotherapeutic agents at the tumor site compared to the bloodstream. In particular, disclosed are self-assembling molecules that transform from isolated molecules or spherical micelles while in blood serum into nanofibers in the acidic extracellular microenvironment of malignant tumor tissue or acidic inflamed tissues. This transition is rapid and reversible, indicating the system is in thermodynamic equilibrium.

The disclosed self-assembling molecules are able to circulate through the vasculature until they encounter an acidic environment. However, since kidneys can also contain an acidic microenvironment, the disclosed self-assembling molecules preferably do not pass through the glomerular basement membrane. Therefore, the self-assembling molecules may have a size and/or charge that reduces glomerular filtration. For example, the self-assembling molecules may have a molecular weight of at least 50 kD, 75 kD, or 100 kD.

In some cases, the self-assembling molecule is conjugated to a macromolecule or particle, such as serum albumin, a polymeric micelle, a liposome, or a polymeric nanoparticle (e.g., a biodegradable polymeric nanoparticle), which due its size and/or charge is excluded from the glomerular filtrate. In other embodiments, the self-assembling molecules are designed to form spherical micelles in blood serum that do not pass through the glomerular basement membrane. Typical micelle sizes are about 10 nm, and the range is from 5 nm to 100 nm.

A composition is therefore disclosed that contains a plurality of biocompatible self-assembling molecules that are isolated molecules or spherical micelles in the neutral pH and isotonic conditions of blood serum, and which transform into cylindrical nanofibers in the acidic extracellular environment of tumors. For example, the plurality of peptide amphiphiles can exist as spherical micelles when in a physiological environment having a pH of 7.30 to 7.45, and transform into cylindrical nanofibers when in a physiological environment having a pH less than 7.3, e.g., environments with a pH of about 5.1 to 7.3, or preferably about 6.4 to 7.3.

At least a portion of the plurality of biocompatible self-assembling molecules are conjugated to a diagnostic or therapeutic agent such that self assembly of the molecules in the acidic environment of a tumor results in accumulation of the diagnostic or therapeutic agent in the tumor.

In some embodiments, the self-assembling molecule contains a peptide amphiphile (or petidomimetic thereof). Peptide amphiphiles are peptide-based molecules that self-assemble into high aspect ratio nanofibers. These molecules typically have three regions: a hydrophobic tail, a region of beta-sheet forming amino acids, and a peptide epitope designed to allow solubility of the molecule in water, perform a biological function by interacting with living systems, or both. Self-assembly occurs by the combination of hydrogen-bonding between beta-sheet forming amino acids and hydrophobic collapse of the tails to yield the formation of spherical micelles or cylindrical nanofibers that present the peptide epitope at extremely high density at the surface.

The term “peptide” refers to any peptide, oligopeptide, polypeptide, or protein. A peptide is comprised of consecutive amino acids. The term encompasses naturally occurring or synthetic amino acids. The term “peptide” includes amino acids joined to each other by peptide bonds or modified peptide bonds, e.g., peptide isosteres, and may contain modified amino acids other than the 20 gene-encoded amino acids. The peptide can be modified by either a natural process, such as post-translational processing, or by chemical modification techniques which are well known in the art. Modifications can occur anywhere in the polypeptide, including the peptide backbone, the amino acid side-chains and the amino or carboxyl termini. The same type of modification can be present in the same or varying degrees at several sites in a given polypeptide. Also, a given peptide can have many types of modifications. Modifications include, without limitation, acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation. GPI anchor formation, hydroxylation, iodination, methylation, myristolyation, oxidation, pegylation, proteolytic processing, phosphorylation, prenylation, racemization, selenoylation, sulfation, and transfer-RNA mediated addition of amino acids to protein such as arginylation.

The term “peptidomimetic” refers to a mimetic of a peptide which includes some alteration of the normal peptide chemistry. Peptidomimetics typically enhance some property of the original peptide, such as increase stability, increased efficacy, enhanced delivery, increased half life, etc. Methods of making peptidomimetics based upon a known polypeptide sequence is described, for example, in U.S. Pat. Nos. 5,631,280; 5,612,895; and 5,579,250. Use of peptidomimetics can involve the incorporation of a non-amino acid residue with non-amide linkages at a given position. Some non-limiting examples of unnatural amino acids which may be suitable amino acid mimics include β-alanine, L-α-amino butyric acid, L-γ-amino butyric acid, L-α-amino isobutyric acid, L-ε-amino caproic acid, 7-amino heptanoic acid, L-aspartic acid, L-glutamic acid, N-ε-Boc-N-α-CBZ-L-lysine, N-ε-Boc-N-α-Fmoc-L-lysine, L-methionine sulfone, L-norleucine, L-norvaline, N-α-Boc-N-δCBZ-L-ornithine, N-δ-Boc-N-α-CBZ-L-ornithine, Boc-p-nitro-L-phenylalanine, Boc-hydroxyproline, and Boc-L-thioproline.

As stated above, the disclosed self-assembling molecules in some embodiments form spherical micelles in the neutral pH and isotonic conditions of blood serum, and transform into cylindrical nanofibers in the acidic extracellular environment of tumors.

A “spherical micelle” is an aggregate of surfactant molecules (e.g., peptide amphiphiles) dispersed in a liquid. A typical micelle in aqueous solution forms an aggregate with the hydrophilic “head” regions in contact with surrounding solvent, sequestering the hydrophobic single-tail regions in the micelle centre. The shape and size of a micelle is a function of the molecular geometry of its surfactant molecules and solution conditions such as surfactant concentration, temperature, pH, and ionic strength.

Micelles only form when the concentration of surfactant is greater than the critical micelle concentration (CMC), and the temperature of the system is greater than the critical micelle temperature, or Kraffit temperature. Micelles can form spontaneously because of a balance between entropy and enthalpy. In water, the hydrophobic effect is the driving force for micelle formation, despite the fact that assembling surfactant molecules together reduces their entropy. At very low concentrations of the lipid, only monomers are present in true solution. As the concentration of the lipid is increased, a point is reached at which the unfavorable entropy considerations, derived from the hydrophobic end of the molecule, become dominant. At this point, the lipid hydrocarbon chains of a portion of the lipids must be sequestered away from the water. Therefore, the lipid starts to form micelles. Broadly speaking, above the CMC, the entropic penalty of assembling the surfactant molecules is less than the entropic penalty of caging the surfactant monomers with water molecules. Also important are enthalpic considerations, such as the electrostatic interactions that occur between the charged parts of surfactants.

The spherical micelles are preferably of a size and charge which allows them to preferentially accumulate in the tumor by the enhanced permeability and retention (EPR), but not be rapidly removed from the bloodstream by glomerular filtration. The EPR effect is a consequence of the abnormal vasculature frequently associated with solid tumors. The vasculature of tumors is typically characterized by blood vessels containing poorly-aligned defective endothelial cells with wider than normal fenestrations. As a result, micelles having an average hydrodynamic diameter of from about 8 nm to about 25 nm can preferentially extravasate from the tumor vasculature, and accumulate within the solid tumor.

Therefore, when present in serum at diagnostically or therapeutically effective concentrations, the disclosed self-assembling molecules preferably form micelles with a hydrodynamic diameter of at least about 8 nm (e.g., at least about 10 nm, at least about 15 nm, at least about 20 nm). In some cases, when present in serum at diagnostically or therapeutically effective concentrations, the disclosed self-assembling molecules preferably form micelles with a hydrodynamic diameter no larger than about 25 nm (e.g., less than about 25 nm, less than about 20 nm, or less than about 15 nm). Dynamic Light Scattering can be used to determine the hydrodynamic diameter of the micelles.

When present in serum at diagnostically or therapeutically effective concentrations, the disclosed self-assembling molecules can form micelles with a hydrodynamic diameter ranging from any of the minimum to any of the maximum diameters described above. For example, the self-assembling molecules can form micelles with a hydrodynamic diameter ranging from about 8 nm to about 25 nm (e.g., from about 8 nm to about 20 nm, or from about 8 nm to about 15 nm).

The spherical micelles or isolated molecules (e.g., bound to a macromolecule) transform into cylindrical nanofibers in the acidic extracellular environment of tumors. The nanofibers are preferably of a size and shape to enhance accumulation within tumor tissue. For example, the cylindrical nanofibers can be greater than about 200 nm, 300 nm, 500 nm, 1000 nm, or 5000 nm in length. In addition, the length of the cylindrical nanofibers may be at least 10 times greater, 20 times greater, or 50 times greater than the diameter of the cylindrical nanofibers, i.e., a length:diameter aspect ratio greater than 10, 20, or 50.

In some embodiments, the self-assembling molecule has three main segments: a hydrophobic alkyl tail, a beta-sheet forming sequence, and a charged sequence. Decreasing the repulsive interaction of the charged region either via electrostatic screening, or by lowering the degree of side-chain ionization with pH, causes these molecules to form nanofibers. By balancing the attractive hydrophobic and hydrogen bonding forces, and repulsive electrostatic and steric forces, the self-assembly morphology and the transition pH can be systematically shifted by tenths of pH values. Moreover, inclusion of sterically bulky agents on the exterior periphery can affect this balance, e.g., by shifting self-assembly to more acidic pH values, and inducing a spherical micellar morphology at high pH and concentration ranges.

The disclosed self-assembling molecules may be designed in such a way that the attractive supramolecular forces (hydrophobic-hydrophobic interactions, beta-sheet formation) and the repulsive supramolecular forces (electrostatic repulsion, sterics) of the molecule are precisely balanced. For example, the repulsive forces can be increased by increasing the number of charged amino acid residues, or adding a unit with larger hydrophilicity or greater steric hindrance, such as a chelating agent. Increasing the attractive forces can be done by using longer alkyl chains, as well as increasing the number of beta-sheet forming residues

In some embodiments, the biocompatible self-assembling molecules can be defined by Formula (I)

Cₙ—Z-A-X (I),

wherein

Cₙ represents an alkyl, alkenyl, or alkynyl group;

Z represents a conjugate comprising Bⁱₒ, Uₚ, Neg_{q}, and optionally Y arranged any order, with the proviso that Bⁱₒ is positioned between Neg_{q} and Cₙ;
wherein Bⁱ, individually for each occurrence, represents an amino acid with intermediate beta-sheet propensity and o represents an integer from 1 to 2, U, individually for each occurrence, represents an uncharged amino acid with poor beta-sheet propensity, and p represents an integer from 0 to 20 (e.g., from 0 to 8),
Neg, individually for each occurrence, represents an anionic amino acid, and wherein q represents an integer from 2 to 7, and
Y is absent, or represents spacer group comprising a diagnostic or therapeutic agent;

A is absent, or represents a hydrophilic linking group; and

X represents a terminating residue,

with the proviso that at least one of Y or A is present in the molecules defined by Formula I.

The amino acids are classified into Table 4.

Cₙ can be an alkyl, alkenyl, or alkynyl group. “Alkyl,” as used herein, refers to the radical of a saturated aliphatic group, including straight-chain alkyl and branched-chain alkyl groups. In some embodiments, the alkyl group comprises 30 or fewer carbon atoms in its backbone (e.g., C₁-C₃₀ for straight chain, C₃-C₃₀ for branched chain). For example, the alkyl group can comprise 25 or fewer carbon atoms, 22 or fewer carbon atoms, 20 or fewer carbon atoms, 19 or fewer carbon atoms, 18 or fewer carbon atoms, 17 or fewer carbon atoms, 16 or fewer carbon atoms, 15 or fewer carbon atoms, 14 or fewer carbon atoms, 12 or fewer carbon atoms, 12 or fewer carbon atoms, 10 or fewer carbon atoms, 8 or fewer carbon atoms, or 6 or fewer carbon atoms in its backbone. In some embodiments, the alkyl group can comprise 6 or more carbon atoms, 8 or more carbon atoms, 10 or more carbon atoms, 11 or more carbon atoms, 12 or more carbon atoms, 13 or more carbon atoms, 14 or more carbon atoms, 15 or more carbon atoms, 16 or more carbon atoms, 17 or more carbon atoms, 18 or more carbon atoms, 19 or more carbon atoms, or 20 or more carbon atoms in its backbone. The alkyl group can range in size from any of the minimum number of carbon atoms to any of the maximum number of carbon atoms described above. For example, the alkyl group can be a C₆-C₃₀ alkyl group (e.g., a C₁₂-C₂₂ alkyl group, or a C₁₂-C₁₈ alkyl group). The term alkyl includes both unsubstituted alkyls and substituted alkyls, the latter of which refers to alkyl groups having one or more substituents, such as a halogen or a hydroxy group, replacing a hydrogen on one or more carbons of the hydrocarbon backbone. The alkyl groups can also comprise between one and four heteroatoms (e.g., oxygen, nitrogen, sulfur, and combinations thereof) within the carbon backbone of the alkyl group. “Alkenyl” and “Alkynyl”, as used herein, refer to unsaturated aliphatic groups containing one or more double or triple bonds analogous in length (e.g., C₂-C₃₀) and possible substitution to the alkyl groups described above.

In certain embodiments, Cₙ is straight-chain C₁₂-C₁₈ alkyl group (e.g., a straight-chain C₁₄-C₁₆ alkyl group). For example, Cₙ can be a lauryl group, a myristyl group, a palmityl group, or a stearyl group.

Bⁱ can be an amino acid with intermediate beta-sheet propensity. Both natural and synthetic amino acids with high beta-sheet propensity are known in the art. Examples of amino acids with intermediate beta-sheet propensity (Bⁱ) include methionine, leucine, threonine, glutamine, tryptophan, and asparagine, as well as synthetic amino acids including L-homoglutamine. Therefore, Bⁱ is not an amino acid with high beta-sheet propensity (B^{s}). Examples of amino acids with high beta-sheet propensity (B^{s}) include isoleucine, phenylalanine, valine, and tyrosine, as well as synthetic amino acids, including phenylglycine and napthyl alanine.

U can be an uncharged amino acid with poor beta-sheet propensity. Both natural and synthetic uncharged amino acids with poor beta-sheet propensity are known in the art. Examples of uncharged amino acids with poor beta-sheet propensity (U) include serine, alanine, and glycine.

Neg can be an anionic amino acid. Anionic amino acids can include amino acids (natural or synthetic) which are negatively charged under physiological conditions. In certain cases, Neg is an amino acid which comprises a side-chain comprising a carboxylic acid moiety. Examples of anionic amino acids include aspartic acid (D) glutamic acid (E), 4-fluoroglutamic acid, and beta-homo-glutamic acid.

In some embodiments, Y can be absent and A can be present. In other embodiments. A can be absent and Y can be present. In some embodiments, both Y and A can be present.

Y can be a spacer group comprising a diagnostic or therapeutic agent. For example, Y can be derived from a divalent molecule comprising a side-chain which includes a therapeutic or diagnostic agent. In certain cases, Y comprises an amino acid having a therapeutic or diagnostic agent covalently attached to the amino acid side-chain.

For example, Y can be derived from an amino acid (natural or synthetic) comprising a side-chain which includes a functional group (e.g., an amine, a carboxylic acid, an aldehyde, an azide, an alkyne, a thiol, an epoxide, or an alcohol). A therapeutic or diagnostic agent (e.g., a chelating agent configured to coordinate a metal ion with diagnostic or therapeutic potential, an aromatic or alkyl entity that can be radiohalogenated) comprising a functional group can be covalently attached to the amino acid via reaction with the functional group in the amino acid side-chain. For example, Y can be lysine conjugated to DO3A.

The therapeutic or diagnostic agent can be directly connected to the amino acid side-chain. In these embodiments, the therapeutic or diagnostic agent comprises a functional group which is reacted with the functional group in the amino acid side-chain, forming a covalent bond between the agent and the amino acid.

In other embodiments, the therapeutic or diagnostic agent can be connected to the amino acid side-chain via a linker. A linker is a divalent chemical group that serves to couple the therapeutic or diagnostic agent to the amino acid side-chain while not adversely affecting either the activity of the agent or the self-assembly of the biocompatible self-assembling molecule. Suitable linking groups include peptides alone, non-peptide groups (e.g., alkyl, alkenyl, or alkynyl groups), or a combination thereof.

For example, the therapeutic or diagnostic agent can be connected to the amino acid side-chain via a linker which includes a C₂-C₁₂ alkyl group, a peptide (e.g., diglycine, triglycine, gly-gly-glu, gly-ser-gly, etc.) in which the total number of atoms in the peptide backbone is less than or equal to twelve, or combinations thereof. In one embodiment, the linker is derived from a substituted alkyl group defined by the formula R₁—(CH₂)ₙ—R₂, wherein n is an integer from 1-10 (e.g., an integer from 3 to 9), R₁ represents a functional group that can be reacted with the functional group in the amino acid side-chain, and R₂ represents a functional group that can form a covalent bond with the therapeutic or diagnostic agent.

As discussed above, in some embodiments, A is absent, in which case Z is directly connected to X. In other embodiments, A is present, and represents a hydrophilic linking group. When the biocompatible self-assembling molecules form micelles in solution, A can be present on the surface of the micelles. In some cases, A is selected so as to provide micelles with prolonged in vivo residence time (e.g., by minimizing uptake of the micelless by the reticuloendothelial system (RES)). For example, A can comprise a hydrophilic oligomer or polymer segment, such as a hydrophilic oligo- or polyalkylene oxide (e.g., oligoethylene glycol or polyethylene glycol (PEG)).

A can comprise a hydrophilic oligo- or polyalkylene oxide having a molecular weight of less than about 5000 Da (e.g., less than 4500 Da, less than about 4000 Da, less than about 3500 Da, less than about 3000 Da, less than about 2500 Da, less than about 2000 Da, less than about 1500 Da, less than about 1000 Da, less than about 800 Da, less than about 750 Da, less than about 600 Da, less than about 500 Da, less than about 450 Da, less than about 400 Da, less than about 350 Da, less than about 300 Da, less than about 250 Da, less than about 200 Da, less than about 150 Da, or less than about 100 Da). A can comprise a hydrophilic oligo- or polyalkylene oxide having a molecular weight of greater than about 50 Da (e.g., greater than about 100 Da, greater than about 150 Da, greater than about 200 Da, greater than about 250 Da, greater than about 300 Da, greater than about 350 Da, greater than about 400 Da, greater than about 450 Da, greater than about 500 Da, greater than about 600 Da, greater than about 750 Da, greater than about 800 Da, greater than about 1000 Da, greater than about 1500 Da, greater than about 2000 Da, greater than about 2500 Da, greater than about 3000 Da, greater than about 3500 Da, greater than about 4000 Da, or greater than about 4500 Da).

A can comprise a hydrophilic oligo- or polyalkylene oxide having a molecular weight ranging from any of the minimum molecular weights to any of the maximum molecular weights described above. For example, A can comprise a hydrophilic oligo- or polyalkylene oxide having a molecular weight ranging from about 50 Da to about 5000 Da (e.g., from about 50 Da to about 1000 Da, from about 50 Da to about 500 Da, from about 100 Da to about 500 Da, from about 500 Da to about 5000, from about 1000 Da to about 5000 Da, from about 1000 Da to about 3000 Da, or from about 1500 Da to about 2500 Da).

In certain embodiments, A comprises a hydrophilic oligoalkylene oxide having a molecular weight of less than about 400 Da. For example, the oligoalkylene oxide can be oligoethylene oxide. In certain embodiments, A can comprise a segment defined by the following formula (—O—CH₂—CH₂—)ᵣ, where r is an integer ranging from 1 to 8.

In certain embodiments, A comprises a hydrophilic polyalkylene oxide having a molecular weight of from about 500 Da to about 5000 Da (e.g., from about 1000 Da to about 5000 Da, or from about 1000 Da to about 3000 Da, or from about 1500 Da to about 2500 Da). For example, the polyalkylene oxide can be polyethylene oxide (PEG). In certain embodiments, A can comprise a segment defined by the following formula (—O—CH₂—CH₂—)ᵣ, where r is an integer ranging from 1 to 150.

In some embodiments, A can comprise an amino acid conjugated to an oligo- or polyalkylene oxide described above. For example, in certain embodiments, A can comprise a lysine residue conjugated to oligoethylene glycol or polyethylene glycol.

X can be any terminating residue. For example, X can be a chemical moiety resulting from the cleavage of the biocompatible self-assembling molecule from a solid support resin used during solid phase peptide synthesis. For example, X can be an amine, an alcohol, an amide group, or a carboxylic acid group (e.g., the NH₂ or COOH group of a C-terminal or N-terminal amino acid). Alternatively, the terminating residue X can be a propionic amide or propionic acid group. X can also be a chemically modified form of such a moiety (e.g., an alkylated amine or an esterified carboxylic acid).

Each of the integers (q, o, p, and n, where is an integer representing the number of carbon atoms in Cₙ) in Formula (I) can be proportionally increased so as to provide larger (i.e., higher molecular weight) self-assembled molecules which can have a similar balance of attractive and repulsive forces. For example, o can represents an integer from 2 to 4, p can represents an integer from 10 to 40, and q can represents an integer from 7 to 14, and n can range from 20 to 40 (e.g., Cₙ represents a C₂₀-C₄₀ alkyl group); or o can represents an integer from 4 to 6, p can represents an integer from 20 to 60, and q can represents an integer from 12 to 21, and n can range from 30 to 60 (e.g., Cₙ represents a C₃₀-C₆₀ alkyl group).

As described above, Z represents a linear conjugate comprising Bⁱₒ, Uₚ, Neg_{q}, and Y arranged any order, with the proviso that Iₒ is positioned between Neg_{q} and Cₙ. In some embodiments, Z can further include one or more additional Bⁱₒ and/or Uₚ segments. For example, Z can be a linear conjugate of Uₚ, Bⁱₒ, Uₚ, Neg_{q}, and Y, or a linear conjugate of Bⁱₒ, Uₚ, Bⁱₒ, Neg_{q}, and Y. The order of Bⁱ to U does not strongly affect the transition. Likewise, the order of Neg to Y does not strongly affect the transition.

In some embodiments, the biocompatible self-assembling molecule is defined by one of the formulae below:

Cₙ—Bⁱₒ—Uₚ-Neg_{q}-Y-A-X (IIA)

Cₙ—Bⁱₒ—Uₚ-Neg_{q}-Y—X (IIB)

Cₙ—Bⁱₒ—Uₚ—Y-Neg_{q}-A-X (IIIA)

Cₙ—Bⁱₒ—Uₚ—Y-Neg_{q}-X (IIIB)

Cₙ—Uₚ—Bⁱₒ—Y-Neg_{q}-A-X (IVA),

Cₙ—Uₚ—Bⁱₒ—Y-Neg_{q}-X (IVB),

Cₙ—Uₚ—Bⁱₒ-Neg_{q}-Y-A-X (VA), or

Cₙ—Uₚ—Bⁱₒ-Neg_{q}-Y—X (VB),

wherein Cₙ, Bⁱ, o, U, p, Y, Neg, q, A, and X are defined as in Formula (I).

In some embodiments, the biocompatible self-assembling molecule is defined by one of the formulae below:

Cₙ—Bⁱₒ—Uₚ-Neg_{q}-A-X (IIC), or

Cₙ—Uₚ—Bⁱₒ-Neg_{q}-A-X (IVC),

wherein Cₙ, Bⁱ, o, U, p, Neg, q, A, and X are defined as in Formula (I).

In some embodiments, the biocompatible self-assembling molecule is defined by one of the formulae below:

(Cₙ)—(BⁱₒUₚ)-(Neg_{q}Y)-(—O—CH₂—CH₂—)ᵣ-propionic amide (VI), or

(Cₙ)—(BⁱₒUₚ)Neg_{q}Y)—NH₂ (VII),

wherein Cₙ, Bⁱ, o, U, p, Y, Neg, and q are defined as in Formula (I). In some cases, Y represents a lysine conjugated to a therapeutic or diagnostic agent (e.g., a DO3A chelating agent optionally bound to a trivalent metal ion), and r represents an integer from 2 to 7. In certain embodiments, the ratio of n:o:q (where n is an integer representing the number of carbon atoms in Cₙ) is 16-17:1:3-4 or 15-16:2:5-7.

In other embodiments, the biocompatible self-assembling molecule comprises the formula:

(Cₙ)—(BⁱₒUₚ)-(Neg_{q-1}Y)-(—O—CH₂—CH₂—)ᵣ-propionic acid (VIII), or

(Cₙ)—BⁱₒUₚ)-(Neg_{q-1}Y)—COOH; (IX),

wherein Cₙ, Bⁱ, o, U, p, Y, Neg, and q are defined as in Formula (I). In some cases, Y represents a lysine conjugated to a therapeutic or diagnostic agent (e.g., aDO3A chelating agent optionally bound to a trivalent metal ion, or a halogenated aromatic or aliphatic), and r represents an integer from 0 to 8. In certain embodiments, the ratio of n:o:q (where n is an integer representing the number of carbon atoms in Cₙ) is 16-17:1:2-3 or 15-16:2:4-6.

In other embodiments, the biocompatible self-assembling molecule comprises the formula:

(Cₙ)—(BⁱₒUₚ)-(Neg_{q})-(—O—CH₂—CH₂—)ᵣ-propionic amide (X)

wherein Cₙ, Bⁱ, o, U, p, Neg, and q are defined as in Formula (I). r can represent an integer from 1 to 150 (e.g., from 50 to 150). In certain embodiments, the ratio of n:o:q (where n is an integer representing the number of carbon atoms in Cₙ) is 16-17:1:2-3 or 15-16:2:4-6.

Also provided are compositions that include a mixture of at least two different biocompatible self-assembling molecules that together assemble to form isolated molecules or spherical micelles in the neutral pH and isotonic conditions of blood serum, and which ransform into cylindrical nanofibers in the acidic extracellular environment of tumors. For example, the mixture of at least two different biocompatible self-assembling molecules can exist as spherical micelles when in a physiological environment having a pH of 7.30 to 7.45, and transform into cylindrical nanofibers when in a physiological environment having a pH less than 7.3, e.g., environments with a pH of about 5.1 to 7.3, or preferably about 6.4 to 7.3.

For example, the mixture of at least two different biocompatible self-assembling molecules can include a plurality of first biocompatible self-assembling molecules that include a therapeutic and/or diagnostic agent and a plurality of second biocompatible self-assembling molecules that include a hydrophilic linking group (e.g., an oligo- or polyalkylene oxide segment, such as a PEG segment). By combining the at least two different biocompatible self-assembling molecules, a mixture having the desired self-assembly properties can be obtained.

For example, the composition can include mixture of at least two different biocompatible self-assembling molecules that a plurality of first biocompatible self-assembling molecules and a plurality of second biocompatible self-assembling molecules. The plurality of first biocompatible self-assembling molecules can be defined by Formula XI

Cₙ-E-A-X (XI),

wherein

Cₙ represents an alkyl, alkenyl, or alkynyl group;

E represents a conjugate comprising Bₒ, Uₚ, Neg_{q}, and Y arranged any order, with the proviso that Bₒ is positioned between Neg_{q} and Cₙ;
wherein B, individually for each occurrence, represents an amino acid with beta-sheet propensity and o represents an integer from 1 to 2,
U, individually for each occurrence, represents an uncharged amino acid with poor beta-sheet propensity, and p represents an integer from 0 to 20,
Neg, individually for each occurrence, represents an anionic amino acid, and wherein q represents an integer from 3 to 7, and
Y represents a spacer group comprising a diagnostic or therapeutic agent;

A is absent, or represents a hydrophilic linking group; and

X represents a terminating residue.

The plurality of second biocompatible self-assembling molecules can be defined by Formula XII

Cₙ—F-A-X (XII)

wherein

Cₙ represents an alkyl, alkenyl, or alkynyl group;

F represents a conjugate comprising Bₒ, Uₚ, Neg_{q}, and optionally Y arranged any order, with the proviso that Bₒ is positioned between Neg_{q} and Cu;
wherein B, individually for each occurrence, represents an amino acid with beta-sheet propensity and o represents an integer from 1 to 2,
U, individually for each occurrence, represents an uncharged amino acid with poor beta-sheet propensity, and p represents an integer from 0 to 20,
Neg, individually for each occurrence, represents an anionic amino acid, and wherein q represents an integer from 3 to 7, and
Y is absent, or represents a spacer group comprising a diagnostic or therapeutic agent;

A represents a hydrophilic linking group; and

X represents a terminating residue.

In these embodiments, Cₙ, o, U, p, Neg, q, Y, A, and X can be as defined above in Formula (I).

B can be, individually for each occurrence, an amino acid with intermediate beta-sheet propensity (Bⁱ) or an amino acid with high beta-sheet propensity (B^{s}). Both natural and synthetic amino acids with intermediate and high beta-sheet propensity are known in the art. Examples of amino acids with intermediate beta-sheet propensity (Bⁱ) include methionine, leucine, threonine, glutamine, tryptophan, and asparagine, as well as synthetic amino acids including L-homoglutamine. Examples of amino acids with high beta-sheet propensity (B^{s}) include isoleucine, phenylalanine, valine, and tyrosine, as well as synthetic amino acids, including phenylglycine and napthyl alanine. In some cases, B can be, individually for each occurrence, an amino acid with intermediate beta-sheet propensity (Bⁱ). In some cases, B can be, individually for each occurrence, an amino acid with high beta-sheet propensity (B^{s}).

In some cases, A is absent from the plurality of first biocompatible self-assembling molecules can be defined by Formula XI. In some cases, Y is absent from the plurality of second biocompatible self-assembling molecules can be defined by Formula XII. In some cases, A is absent from the plurality of first biocompatible self-assembling molecules can be defined by Formula XI and Y is absent from the plurality of second biocompatible self-assembling molecules can be defined by Formula XII.

In some embodiments, the plurality of first biocompatible self-assembling molecules are defined by one of formulae below

Cₙ—Bₒ—Uₚ-Neg_{q}-Y-A-X (XIIIA)

Cₙ—Bₒ—Uₚ-Neg_{q}-Y—X (XIIIB)

Cₙ—Bₒ—Uₚ—Y-Neg_{q}-A-X (XIVA)

Cₙ—Bₒ—Uₚ—Y-Neg_{q}-X (XIVB)

Cₙ—Uₚ—Bₒ—Y-Neg_{q}-A-X (XVA),

Cₙ—Uₚ—Bₒ—Y-Neg_{q}-X (XVB),

Cₙ—Uₚ—Bₒ-Neg_{q}-Y-A-X (XVIA), or

Cₙ—Uₚ—Bₒ-Neg_{q}-Y—X (XVIB),

wherein Cₙ, B, o, U, p, Neg, q, Y, A, and X are as defined in Formula (XI).

In some embodiments, the plurality of second biocompatible self-assembling molecules are defined by one of formulae below

Cₙ—Bₒ—Uₚ-Neg_{q}-A-X (XVI), or

Cₙ—Uₚ—Bₒ-Neg_{q}-A-X (XVII),

wherein Cₙ, B, o, U, p, Neg, q, A, and X are as defined in Formula (XII).

In some embodiments, the plurality of first biocompatible self-assembling molecules are defined by one of formulae below

(Cₙ)—(BₒUₚ)-(Neg_{q}Y)-(—O—CH₂—CH₂—)ᵣ-propionic amide (XVIII), or

(Cₙ—(BₒUₚ)-(Neg_{q}Y)—NH₂ (XIX),

wherein Cₙ, B, o, U, p, Y, Neg, and q are defined as in Formula (XI). In some cases, Y represents a lysine conjugated to a therapeutic or diagnostic agent (e.g., a DO3A chelating agent optionally bound to a trivalent metal ion), and r represents an integer from 2 to 7. In certain embodiments, the ratio of n:o:q (where n is an integer representing the number of carbon atoms in Cₙ) is 16-17:1:3-4 or 15-16:2:5-7.

In some embodiments, the plurality of first biocompatible self-assembling molecules are defined by one of formulae below

(Cₙ)—(BₒUₚ)-(Neg_{q-1}Y)-(—O—CH₂—CH₂—)ᵣ-propionic acid (XX), or

(Cₙ)—(BₒUₚ)—(Neg_{q-1}Y)—COOH (XXI),

wherein Cₙ, Bⁱ, o, U, p, Y, Neg, and q are defined as in Formula (XI). In some cases, Y represents a lysine conjugated to a therapeutic or diagnostic agent (e.g., aDO3A chelating agent optionally bound to a trivalent metal ion, or a halogenated aromatic or aliphatic), and r represents an integer from 0 to 8. In certain embodiments, the ratio of n:o:q (where n is an integer representing the number of carbon atoms in Cₙ) is 16-17:1:2-3 or 15-16:2:4-6.

In some embodiments, the plurality of second biocompatible self-assembling molecules are defined by one of formulae below

(Cₙ)—(BₒUₚ)-(Neg_{q})-(—O—CH₂—CH₂—)ᵣ-propionic amide (XXII)

wherein Cₙ, B, o, U, p, Neg, and q are defined as in Formula (XII). r can represent an integer from 1 to 150 (e.g., from 50 to 150). In certain embodiments, the ratio of n:o:q (where n is an integer representing the number of carbon atoms in Cₙ) is 16-17:1:2-3 or 15-16:2:4-6.

In the case of the mixtures described above, the weight ratio of first biocompatible self-assembling molecules to second biocompatible self-assembling molecules in the mixture can vary. In some embodiments, the weight ratio of first biocompatible self-assembling molecules to second biocompatible self-assembling molecules in the mixture can be from 2:1 to 20:1 (e.g., from 3:1 to 19:1).

The disclosed self-assembling molecules can contain diagnostic or therapeutic agents for detecting and/or treating tissue where the self-assembling molecules accumulate, e.g., malignant tumors or inflamed joints. The diagnostic or therapeutic agent can be any molecule suitable for molecular imaging or targeted tumor therapy, respectively. In some embodiments, the diagnostic agent is a molecule detectable in the body of a subject by an imaging technique such as X-ray radiography, ultrasound, computed tomography (CT), single-photon emission computed tomography (SPECT), magnetic resonance imaging (MRI), positron emission tomography (PET), Optical Fluorescent Imaging, Optical Visible light imaging, and nuclear medicine including Cerenkov Light Imaging. For example, the diagnostic agent can comprise a radionuclide, paramagnetic metal ion, or a fluorophore.

The terms “metal chelator” and “chelating agent” refer to a polydentate ligand that can form a coordination complex with a metal atom. It is generally preferred that the coordination complex is stable under physiological conditions. That is, the metal will remain complexed to the chelator in vivo.

In some cases, the metal chelator is a molecule that complexes to a radionuclide metal or paramagnetic metal ion to form a metal complex that is stable under physiological conditions.

The metal chelator may be any of the metal chelators known in the art for complexing a medically useful paramagnetic metal ion, or radionuclide.

In some cases, such as in the case of self-assembling molecules designed for radiopharmaceutical or radiotherapy applications, it can be convenient to prepare the complexes comprising a radionuclide, at or near the site where they are to be used (e.g., in a hospital pharmacy or clinic). Accordingly, in some embodiments, the self-assembling molecule comprises a metal chelator uncomplexed with a metal ion. In such embodiments, the self-assembling molecule can be complexed with a suitable metal ion prior to administration. In other embodiments, the self-assembling molecule comprises a metal chelator complexed with a suitable metal ion (e.g., a paramagnetic metal ion or a radionuclide).

Suitable metal chelators include, for example, linear, macrocyclic, terpyridine, and N₃S, N₂S₂, or N₄ chelators (see also, U.S. Pat. No. 4,647,447, U.S. Pat. No. 4,957,939, U.S. Pat. No. 4,963,344, U.S. Pat. No. 5,367,080, U.S. Pat. No. 5,364,613, U.S. Pat. No. 5,021,556, U.S. Pat. No. 5,075,099, U.S. Pat. No. 5,886,142, the disclosures of which are incorporated by reference herein in their entirety), and other chelators known in the art including, but not limited to, HYNIC, DTPA, EDTA, DOTA, TETA, and bisamino bisthiol (BAT) chelators (see also U.S. Pat. No. 5,720,934). For example, macrocyclic chelators, and in particular N₄ chelators are described in U.S. Pat. Nos. 4,885,363; 5,846,519; 5,474,756; 6,143,274; 6,093,382; 5,608,110; 5,665,329; 5,656,254; and 5,688,487, the disclosures of which are incorporated by reference herein in their entirety. Certain N₃S chelators are described in PCT/CA94/00395, PCT/CA94/00479, PCT/CA95/00249 and in U.S. Pat. Nos. 5,662,885; 5,976,495; and 5,780,006, the disclosures of which are incorporated by reference herein in their entirety. The chelator may also include derivatives of the chelating ligand mercapto-acetyl-glycyl-glycyl-glycine (MAG3), which contains an N₃S, and N₂S₂ systems such as MAMA (monoamidemonoaminedithiols), DADS (N₂S diaminedithiols). CODADS and the like. These ligand systems and a variety of others are described in Liu and Edwards, Chem. Rev. 1999, 99, 2235-2268; Caravan et al., Chem. Rev. 1999, 99, 2293-2352; and references therein, the disclosures of which are incorporated by reference herein in their entirety.

The metal chelator may also include complexes known as boronic acid adducts of technetium and rhenium dioximes, such as those described in U.S. Pat. Nos. 5,183,653; 5,387,409; and 5,118,797, the disclosures of which are incorporated by reference herein, in their entirety.

Examples of suitable chelators include, but are not limited to, derivatives of diethylenetriamine pentaacetic acid (DTPA), 1,4,7,10-tetraazacyclotetradecane-1,4,7,10-tetraacetic acid (DOTA), 1-substituted 1,4,7,-tricarboxymethyl 1,4,7,10 tetraazacyclododecane triacetic acid (DO3A), derivatives of the 1-1-(1-carboxy-3-(p-nitrophenyl)propyl-1,4,7,10 tetraazacyclododecane triacetate (PA-DOTA) and MeO-DOTA, ethylenediaminetetraacetic acid (EDTA), and 1,4,8,11-tetraazacyclotetradecane-1,4,8,11-tetraacetic acid (TETA), derivatives of 3,3,9,9-Tetramethyl-4,8-diazaundecane-2, 10-dione dioxime (PnAO); and derivatives of 3,3,9,9-Tetramethyl-5-oxa-4,8-diazaundecane-2,10-dione dioxime (oxa PnAO). Additional chelating ligands are ethylenebis-(2-hydroxy-phenylglycine) (EHPG), and derivatives thereof, including 5-C1-EH PG, 5-Br-EH PG, 5-Me-EH PG, 5-t-Bu-EH PG, and 5-sec-Bu-EHPG; benzodiethylenetriamine pentaacetic acid (benzo-DTPA) and derivatives thereof, including dibenzo-DTPA, phenyl-DTPA, diphenyl-DTPA, benzyl-DTPA, and dibenzyl-DTPA; bis-2 (hydroxybenzyl)-cthylene-diaminediacetic acid (HBED) and derivatives thereof; the class of macrocyclic compounds which contain at least 3 carbon atoms and at least two heteroatoms (O and/or N), which macrocyclic compounds can consist of one ring, or two or three rings joined together at the hetero ring elements, e.g., benzo-DOTA, dibenzo-DOTA, and benzo-NOTA, where NOTA is 1,4,7-triazacyclononane N,N′,N″-triacetic acid, benzo-TETA, benzo-DOTMA, where DOTMA is 1,4,7,10-tetraazacyclotetradecane-1,4,7,10-tetra(methyl tetraacetic acid), and benzo-TETMA, where TETMA is 1,4,8,11-tetraazacyclotetradecane-1,4,8,11-(methyl tetraacetic acid); derivatives of 1,3-propylenediaminetetraacetic acid (PDTA) and triethylenetetraaminehexaacetic acid (TTHA); derivatives of 1,5,10-N,N′,N″-tris(2,3-dihydroxybenzoyl)-tricatecholate (LICAM) and 1,3,5-N,N′,N″-tris(2,3-dihydroxybenzoyl)aminomethylbenzene (MECAM). Examples of representative chelators and chelating groups are described in WO 98/18496, WO 86/06605, WO 91/03200, WO 95/28179, WO 96/23526, WO 97/36619, PCT/US98/01473, PCT/US98/20182, and U.S. Pat. No. 4,899,755, U.S. Pat. No. 5,474,756, U.S. Pat. No. 5,846,519 and U.S. Pat. No. 6,143,274, each of which is hereby incorporated by reference in its entirety.

In some embodiments, the metal chelator comprises desferrioxamine (also referred to as deferoxamine, desferrioxamine B, desferoxamine B, DFO-B, DFOA, DFB or desferal) or a derivative thereof. See, for example U.S. Pat. No. 8,309,583, U.S. Pat. No. 4,684,482, and U.S. Pat. No. 5,268,165, each of which is hereby incorporated by reference in its entirety for its teaching of desferrioxamine and desferrioxamine derivatives.

As is well known in the art, metal chelators can be specific for particular metal ions. Suitable metal chelators can be selected for incorporation into the self-assembling molecule based on the desired metal ion and intended use of the self-assembling molecule.

Paramagnetic ions form a magnetic moment upon the application of an external magnetic field thereto. Magnetization is not retained in the absence of an externally applied magnetic field because thermal motion causes the spin of unpaired electrons to become randomly oriented in the absence of an external magnetic field. By taking advantage of its property of shortening the magnetic relaxation time of water molecules, a paramagnetic substance is usable as an active component of MRI contrast agents. Suitable paramagnetic transition metal ions include Cr³⁺, Co²⁺, Mn²⁺, Ni²⁺, Fe²⁺, Fe³⁺, Zr⁴⁺, Cu²⁺, and Cu³⁺. In preferred embodiments, the paramagnetic ion is a lanthanide ion (e.g., La³⁺, Gd³⁺, Ce³⁺, Tb³⁺, Pr³⁺, Dy³⁺, Nd³⁺, Ho³⁺, Pm³⁺, Er³⁺, Sm³⁺, Tm³⁺, Eu³⁺, Yb³⁺, or Lu³⁺). In MRI, especially preferred metal ions are Gd³⁺, Mn²⁺, Fe³⁺, and Eu²⁺.

MRI contrast agents can also be made with paramagnetic nitroxides molecules in place of the chelating agent and paramagnmetic metal ion.

Suitable radionuclides include ^{99m}Tc, ⁶⁷Ga, ⁶⁸Ga, ⁶⁶Ga, ⁴⁷Sc, ⁵¹Cr, ¹⁶⁷Tm, ¹⁴¹Ce, ¹¹¹In, ¹²³I, ¹²⁵I, ¹³¹I, 124I, ¹⁸F, ¹¹C, ¹⁵N, 17O, ¹⁶⁸Yb, ¹⁷⁵Yb, ¹⁴⁰La, ⁹⁰Y, ⁸⁸S, ⁸⁶Y, ¹⁵³Sm, ¹⁶⁶Ho, ¹⁶⁵Dy, ¹⁶⁶Dy, ⁶²Cu, ⁶⁴Cu, ⁶⁷Cu, ⁹⁷Ru, ¹⁰³Ru, ¹⁸⁶Re, 1⁸⁸Re, ²⁰³Pb, ²¹¹Bi, ²¹²Bi, ²¹³Bi, ²¹⁴Bi, ²²⁵Ac, ²¹¹At, ¹⁰⁵Rh, ¹⁰⁹Pd, ^{117m}Sn, ¹⁴⁹Pm, ¹⁶¹Tb, ¹¹⁷Lu, ¹⁹⁸Au, ¹⁹⁹Au, 89Zr, and oxides or nitrides thereof. The choice of isotope will be determined based on the desired therapeutic or diagnostic application. For example, for diagnostic purposes (e.g., to diagnose and monitor therapeutic progress in primary tumors and metastases), suitable radionuclides include ⁶⁴Cu, ⁶⁷Ga, ⁶⁸Ga, ⁶⁶Ga, ^{99m}Tc, and ¹¹¹In, ¹⁸F, ⁸⁹Zr, ¹²³I, ¹³¹I, ¹²⁴I, ¹⁷⁷Lu, ¹⁵N, ¹⁷O. For therapeutic purposes (e.g., to provide radiotherapy for primary tumors and metastasis related to cancers of the prostate, breast, lung, etc.), suitable radionuclides include ⁶⁴Cu, ⁹⁰Y, ¹⁰⁵Rh, ¹¹¹In, 131I, ^{117m}Sn, ¹⁴⁹Pm, ¹⁵³Sm, ¹⁶¹Tb, ¹⁶⁶Dy, ¹⁶⁶Ho, ¹⁷⁵Yb, ¹⁷⁷Lu, ^{186/188}Re, ¹⁹⁹Au, ¹³¹I, and ¹²⁵I, ²¹²Bi, ²¹¹At.

In the case of self-assembled molecules designed to be imaged using PET, radionuclides with short half-lives such as carbon-11 (˜20 min), nitrogen-13 (˜10 min), oxygen-15 (˜2 min), fluorine-18 (˜110 min)., or rubidum-82 (˜1.27 min) are often used. In certain embodiments when a non-metal radionuclide is employed, the therapeutic or diagnostic agent comprises a radiotracer covalently attached to the self-assembling molecule. By way of exemplification, suitable ¹⁸F-based radiotracers include ¹⁸F-fluordesoxyglucose (FDG), ¹⁸F-dopamine, ¹⁸F-L-DOPA, ¹⁸F-fluorcholine, ¹⁸F-fluormethylethylcholin, and ¹⁸P-fluordihydrotestosteron.

In the case of self-assembled molecules designed to be imaged using PET, radionuclides with long half-lives such as ¹²⁴I, or ⁸⁹Zr are also often used.

Fluorescent imaging has emerged with unique capabilities for molecular cancer imaging. Fluorophores emit energy throughout the visible spectrum; however, the best spectrum for in vivo imaging is in the near-infrared (NIR) region (650 nm-900 nm). Unlike the visible light spectrum (400-650 nm), in the NIR region, light scattering decreases and photo absorption by hemoglobin and water diminishes, leading to deeper tissue penetration of light. Furthermore, tissue auto-fluorescence is low in the NIR spectra, which allows for a high signal to noise ratio. There is a range of small molecule organic fluorophores with excitation and emission spectra in the NIR region. Some, such as indocyanine green (ICG) and cyanine derivatives Cy5.5 and Cy7, have been used in imaging for a relatively long time. Modern fluorophores are developed by various biotechnology companies and include: Alexa dyes; IRDye dyes; VivoTag dyes and HylitePlus dyes. In general, the molecular weights of these fluorophores are below 1 kDa.

In some embodiments, the therapeutic or diagnostic agent comprises a radiocontrast agent. In these embodiments the therapeutic agent can comprise an iodinated moiety covalently attached to the self-assembling molecule. Examples of suitable radiocontrast agents include iohexol, iodixanol and ioversol.

Disclosed are pharmaceutical compositions containing therapeutically effective amounts of one or more of the disclosed self-assembling molecules, or mixture of moleduces, and a pharmaceutically acceptable carrier. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio. Pharmaceutical carriers suitable for administration of the molecules provided herein include any such carriers known to those skilled in the art to be suitable for the particular mode of administration.

In some cases, formulations contain exclusively one type of self-assembling molecule. In other cases, the formulations include a mixture of two or more self-assembling molecules. For example, in some embodiments, the formulation contains a portion of self-assembling molecules bound to diagnostic agents and a portion that is free of diagnostic agents. The optimal ratio of bound and unbound molecules can be determined empirically by ordinary skill.

The self-assembling molecules can be formulated for a variety of routes of administration and/or applications. For use in conjunction with the treatment and/or diagnosis of tumors, the self-assembling molecules are preferably administered by injection intravenously or intraparentoneally for tumor imaging. The self-assembling molecules can also be administered by alternative parenteral routes which are suitable to achieve tumor localization and self-assembly. For example, the self-assembling molecules can be administered into and/or around a tumor in, for example, sentinel lymph node identification. A non tumor example would be intrasynovial administration to evaluate inflammation in inflamed acidic joint spaces Subcutaneous administration could be used to evaluate the tumorogenic status of lymph nodes.

Suitable dosage forms for parenteral administration include solutions, suspensions, and emulsions. Typically, the self-assembling molecules are dissolved or suspended in a suitable solvent such as, for example, water, Ringer's solution, phosphate buffered saline (PBS), or isotonic sodium chloride. The formulation may also be a sterile solution, suspension, or emulsion in a nontoxic, parenterally acceptable diluent or solvent such as 1,3-butanediol.

Formulations may further include one or more additional excipients. Representative excipients include solvents, diluents, pH modifying agents, preservatives, antioxidants, antinfective agents, suspending agents, wetting agents, viscosity modifiers, tonicity agents, stabilizing agents, and combinations thereof. Suitable pharmaceutically acceptable excipients are preferably selected from materials which are generally recognized as safe (GRAS), and may be administered to an individual without causing undesirable biological side effects or unwanted interactions.

In some cases, formulations can include one or more tonicity agents to adjust the isotonic range of the formulation. Suitable tonicity agents are well known in the art and include glycerin, mannitol, sorbitol, sodium chloride, and other electrolytes.

In some cases, the formulations can be buffered with an effective amount of buffer necessary to maintain a pH suitable for parenteral administration. Suitable buffers are well known by those skilled in the art and some examples of useful buffers are acetate, borate, carbonate, citrate, and phosphate buffers.

In some instances, the formulation is distributed or packaged in a liquid form. Alternatively, formulations for ocular administration can be packed as a solid, obtained, for example by lyophilization of a suitable liquid formulation. The solid can be reconstituted with an appropriate carrier or diluent prior to administration.

If desired, formulations can contain one or more radiostabilizers to slow or prevent radiolytic damage to components of the composition. Formulations may be liquid or in lyophilized form using lyophilation agents such as sorbitol or mannitol, and such agents would be redissolved in water for injection, dextrose, saline or phosphate buffered saline or other suitable injectable, sterile liquid. Injectable formulation of these self assembling diagnostic or therapeutic self assembling molecules can be made sterile and pyrogen free by methods known in the pharmaceutical art.

The disclosed self-assembling molecules that accumulate within acid tissue, such as tumors or inflamed joints, may be used to diagnose or treat a condition characterized by the acid tissue (e.g., tumors or inflammation) in subjects. Therefore, disclosed is a method for diagnosing cancer in a subject that involves first administering to the subject an effective amount of a composition containing a plurality of the disclosed biocompatible self-assembling molecules conjugated to a diagnostic agent, and then imaging the subject for the presence of the diagnostic agent, wherein detection of an accumulated amount of the diagnostic agent in the subject is an indication of the presence of a tumor.

The term “accumulated amount” generally refers to an amount sufficient detect the diagnostic agent against background levels. For example, a concentration of diagnostic agent at least 20%, 30%, 40%, 50%, 60%, 70%, 80%, 90%, or 100% higher than background levels can be sufficient for detection.

Imaging technologies are known in the art and include without limitation X-ray radiography, ultrasound, computed tomography (CT), single-photon emission computed tomography (SPECT), magnetic resonance imaging (MRI), positron emission tomography (PET), Optical imaging and nuclear medicine. When the appropriate diagnostic agent is present in the disclosed self-assembling molecules, these technologies may be used to detect accumulated self-assembling molecules within tumors.

Also disclosed is a method for treating cancer in a subject that involves administering to the subject a composition containing a plurality of the disclosed biocompatible self-assembling molecules conjugated to a therapeutic agent, wherein therapeutic agent accumulates in the cancer of the subject in a therapeutically effective amount and treats the cancer.

For example, the therapeutic agent can comprises a radionuclide suitable for targeted radionuclide tumor therapy. In targeted radionuclide therapy, the biological effect is obtained by energy absorbed from the radiation emitted by the radionuclide. Whereas the radionuclides used for nuclear medicine imaging emit gamma rays, which can penetrate deeply into the body, the radionuclides used for targeted radionuclide therapy must emit radiation with a relatively short path length. There are three types of particulate radiation of consequence for targeted radionuclide therapy-beta particles, alpha particles, and Auger electrons, which can irradiate tissue volumes with multicellular, cellular and subcellular dimensions, respectively. In some cases, mixed emitters are used to allow both imaging and therapy with the same radionuclide (e.g., the mixed beta/gamma emitter, iodine-131 and ¹⁷⁷Lu). Moreover, within each of these categories, there are multiple radionuclides with a variety of tissue ranges, half-lives, and chemistries, offering the attractive possibility of tailor-making the properties of a targeted radionuclide therapeutic to the needs of an individual patient.

The range of alpha particles in tissue is only a few cell diameters, offering the prospect of matching the cell-specific nature of molecular targeting with radiation of a similar range of action. Another attractive feature of alpha particles for targeted radionuclide therapy is that, as a consequence of their high linear energy transfer, they may have greater biological effectiveness per nuclide than either conventional external beam x-ray radiation or beta emitters. Studies performed in cell culture have demonstrated that human cancer cells can be killed even after being hit by only a few alpha particles and that unlike other types of radiation, where oxygen is necessary for free radicals to be generated, efficient cancer cell elimination can be achieved even in an hypoxic environment. Phase I clinical trials have been performed with bismuth-213- and astatine-211-labeled monoclonal antibodies in patients with leukemia and brain tumors, respectively, and radium-223 is being evaluated in breast and prostate cancer patients with bone metastases.

Currently, the targeted radiotherapeutics approved by the FDA for human use are limited to four beta emitters: yttrium-90 and iodine-131, which are used in tandem with monoclonal antibodies to treat non-Hodgkin's lymphoma, and samarium-153-EDTMP (Quadramett®) and strontium-89-chloride for palliation of bone metastases. However, the scope of preclinical and clinical research in the therapy field is much broader, involving at least eight additional beta-emitting radionuclides: lutetium-177, holmium-166, rhenium-186, rhenium-188, copper-67, promethium-149, gold-199, and rhodium-105.

Auger electron emitters, such as bromine-77, indium-111, iodine-123, and iodine-125, may also be used for radiotherapy. When used in concert with targeting vehicles that can localize these subcellular-range radiations in close proximity to cellular DNA, studies in cell culture have shown highly effective and specific tumor cell killing.

In some embodiments, the method further comprises administering to the subject a composition containing a radiosensitizer. Examples of known radiosensitizers include gemcitabine, 5-fluorouracil, pentoxifylline, and vinorelbine.

In some embodiments, the self-assembling molecule comprises a metal chelator uncomplexed with a metal ion. In such embodiments, methods may further involve complexing the metal chelator with a suitable metal ion prior to administration.

The tumor of the disclosed methods can be any tissue in a subject undergoing unregulated growth, invasion, or metastasis, and having a relatively acidic extracellular microenvironment. Most cancers heavily use glycolytic metabolism to a greater extent than do normal tissues. Glycolytic metabolism produces excess protons and lactic acid in the extracellular spaces of the tumor and its immediate surroundings, which lowers the pH from physiologic 7.4. Generally, the more aggressive cancers produce greater quantities of acid and lower extracellular pH environments. Therefore, in some embodiments, the tumor is any tissue that preferentially uptakes fluorodeoxyglucose (¹⁸F-FDG). For example, the tumor can be Hodgkin's disease, non-Hodgkin's lymphoma, colorectal cancer, breast cancer, renal cancer, melanoma, or lung cancer.

In some embodiments, the cancer is prostate cancer, which does not have preferential uptake of ¹⁸F-FDG.

In some aspects, the tumor of the disclosed methods is a neoplasm for which radiotherapy is currently used. The tumor can also be a neoplasm that is not sufficiently sensitive to radiotherapy using standard methods. The tumor can be a sarcoma, lymphoma, carcinoma, blastoma, or germ cell tumor. A representative but non-limiting list of cancers that the disclosed compositions can be used to treat include B cell lymphoma. T cell lymphoma, mycosis fungoides, Hodgkin's Disease, bladder cancer, brain cancer, nervous system cancer, head and neck cancer, squamous cell carcinoma of head and neck, kidney cancer, lung cancers such as small cell lung cancer and non-small cell lung cancer, adenocarcinoma, liposarcoma, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

The exact amount of the compositions administered to a patient will vary from subject to subject, depending on the nature of the diagnostic or therapeutic agent (e.g., type of imaging employed, nature of the agent, etc.), the species, age, weight and general condition of the subject, the mode of administration and the like. It will also depend on the imaging modality for which the invention has been constructed. Doses for diagnostic imaging are generally in decreasing order: X ray>MRI >Optical >nuclear. For example, X-ray imaging can involve accumulating about 1-2 mM iodine at the tumor site. MRI can be approximately 10 times lower. Optical Fluorescence imaging can be about 5-10 times lower than MRI, and nuclear mass doses can be lower than nuclear, and dependent mostly on the nuclear radioactive dose rather than the mass dose.

For example, a self assembling diagnostic agent for MRI can contain a chelating agent which is bound tightly to a paramagnetic metal such as Gd³⁺. In this mode the dose of the agent can be about 0.025-0.3 mmol/kg. When imaging will be via PET using ⁶⁸Ga as the positron emitting nuclide, the chelating agent could again be used, optionally adjusted for the size difference between Ga³⁺ and Gd³⁺, and the radioactive dose could be about 2-5 mCi for a human 70 kg patient. Veterinary dosing would depend primarily on the weight of the veterinary patient, with, for example, a 70 kg porcine patient receiving about the same dose as a 70 kg human.

In another embodiment, nuclear medicine diagnostics are performed using ¹⁸F or ¹²⁴I nuclides. In these cases, the chelating agent can be replaced with an aliphatic, or aromatic group, respectively, for standard radiolabeling with these halogens, respectively. The dosage for imaging with PET can be approximately similar to dosage used for ⁶⁸Ga. For radiotherapy, a self-assembling molecule using a metal chelator, for example to chelate ¹⁷⁷Lu, can be delivered in monthly doses of an empirically determined amount which spares (or minimizes the damage to) normal tissues but otherwise was maximized for tumor killing. The target organ for these self assembling molecules can include bone marrow, liver and GI systems. Maximal human single doses can be as high as possible, but at least 50 mCi/month, and preferably up to 300 mCi/month. Generally in nuclear medicine diagnostics and therapeutics, the mass dose (mass/kg) is lower than in non-nuclear imaging such as X ray, MRI and Optical imaging. See, for example, Sovak M. ed. Radiocontrast Agents. New York: Springer-Verlag, 1984: Handbook of Experimental Pharmacology Volume 73. 1984; Tweedle M F. Relaxation Agents in NMR Imaging. In J.-C. G. Bunzli, G. R., ed. Lanthanide Probes in Life, Chemical and Earth Sciences, Theory and Practice. Amsterdam: Elsevier, 1989: 127-179; Merbach A E; Toth E; eds. The Chemistry of Contrast Agents in Medical Magnetic Resonance Imaging. Wiley, 2001. Nuclear: Sandler M P, Coleman R E, Patton J A, Wackers F J th, Gottschalk A, eds. Diagnostic Nuclear Medicin, 4^{th} Edition, Lippincott Willliams Wilkins. 2003. And in Radiotherapy, Speer T W; Targeted Radionuclide therapy, Wolters Kluwer, 2011.

### Optical Azar FS, Intes X; Translational Multimodality Optical Imaging, Artech House, 2008.

Effective dosages and schedules for administering the compositions may be determined empirically, and making such determinations is within the skill in the art. The dosage ranges for the administration of the compositions are those large enough to produce the desired effect (e.g., a therapeutic result or a suitable diagnostic result). The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications.

### EXAMPLES

### Example 1: Fine-Tuning the pH Trigger of Self-Assembly

There has been much interest in understanding the influence of size, shape, and mechanical properties of nanomaterials on their biodistribution, to design more effective drug delivery and imaging agents. For example, the enhanced permeation and retention of spherical materials with 20-200 nm diameters in the leaky, non-lymphatic vasculature of tumor tissue has ultimately led to the development of FDA-approved liposomal therapies (Torchilin, V. P. Nat. Rev. Drug Discov. 2005 4:145-60; Matsumura, Y. et al. Cancer Res. 1986 46:6387-92). More recently, the size and shape of nanomaterials has been found to play a significant role in the distribution and circulation lifetimes of these objects when delivered intraveneously (Geng, Y., et al. Nat. Nanotech. 2007 2:249-55; Petros, R. A., et al. Nat. Rev. Drug Discov. 2010 9:615-27; Yoo, J.-W., et al. Nat. Rev. Drug Discov. 2011 10:521-35; Popovic, Z., et al. Angew. Chem. Int. Ed. 2010, 49, 8649-52). For example, cylindrical polymeric micelles have been shown to have a ten times longer circulation time in the bloodstream compared to their spherical counterparts (Geng, Y., et al. Nat. Nanotech. 2007 2:249-55). Still, most of these materials tend to be either static objects that do not transform in the cancer environment or carriers that fragment into smaller objects to release cargo when they get to the target (Sawant, R. M., et al. Bioconjugate Chem. 2006 17:943-49; Torchilin, V. P. Pharm. Res. 2007 24: 1-16).

Designing nanomaterials that can spontaneously change shape and size in response to specific physiological stimuli has the potential to exploit the differential diffusion kinetics to amplify the accumulation of these agents. For cancer, one particularly attractive stimulus is the slightly acidic extracellular microenvironment of tumor tissue (pH 6.6-7.4) (Gatenby. R. A., et al. Nat Rev Cancer 2004 4:891-99) that arises due to the enhanced rate of glycolysis (Hanahan, D., et al. Cell 2011 144:646-74). There are numerous examples of materials that incorporate acid-cleavable linkages that degrade under the lysosomal (pH 5.0-5.5) or the slightly acidic tumor environment to release cargo (Sawant, R. M., et al. Bioconjugate Chem. 2006 17:943-49; Torchilin, V. P. Pharm. Res. 2007 24: 1-16), however, there are far fewer examples of materials that reversibly transform to larger, more slowly diffusing morphologies in response to the extracellular cancer pH. The notion of creating a material that, upon reaching the acidic extracellular tumor environment, transforms into a bulky, more slowly diffusing object could serve as a mechanism for achieving a higher relative concentration of imaging, drug delivery, or radiotherapeutic agent at the tumor site compared to the bloodstream. Although a multitude of self-assembling materials have pH-dependent assembly behavior, there are very few biologically compatible systems designed for in vivo use, with assembly behavior that can be reversibly triggered at neutral pH values (6.6-7.4) in an ionic environment that resembles serum. Both the concentration and the valency of the ionic environment plays a key role in mediating the self-assembly of charged systems (Hiemenz, P. C., et al. Rajagopalan, R. Principles of Colloid and Surface Chemistry; 3rd ed., 1997). Thus, developing systems that function under the stringent set of conditions for in vivo use requires a considerable amount of insight and optimization.

Results

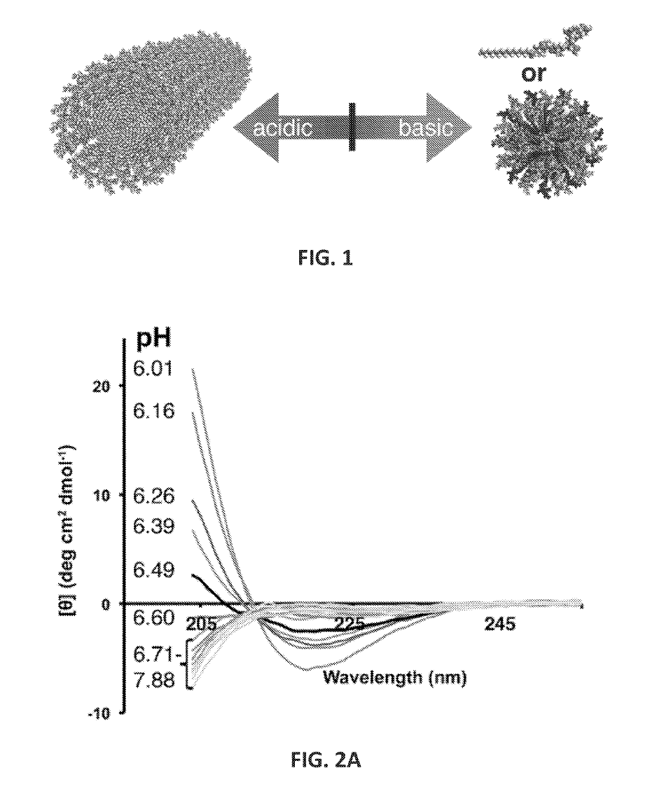

To develop materials capable of reversible pH-triggered morphological changes, amphiphilic molecules that exist as either single molecules or spherical micelles under normal physiological conditions (pH=7.4) but self-assemble into nanofibers upon encountering the acidic environment (pH=6.6) of the tumor vasculature were designed (FIG. 1). Peptide amphiphiles (PA) (Table 1) are an attractive class of molecules in this regard since they are biocompatible, can spontaneously self-assemble into a variety of morphologies, and their intermolecular forces can be precisely tuned with the peptide sequence (Cui, H., et al. Biopolymers 2010 94:1-18; Missirlis, D., et al. Langmuir 2011 27:6163-70; Paramonov, S. E., et al. J. Am. Chem. Soc. 2006 128:7291-98; Goldberger, J. E., et al. Angew. Chem. Int. Ed. 2011 50:6292-95). The designed PA molecules consisted of three main segments: a hydrophobic alkyl tail, a β-sheet forming peptide sequence, and a charged amino acid sequence. Decreasing the repulsive interaction of the charged region either via electrostatic screening, or by lowering the degree of side-chain ionization with pH, causes these molecules to form nanofibers. By balancing the relative attractive and repulsive forces via the peptide sequence it is possible to enable the transition to occur at the desired pH in physiological salt concentrations.

TABLE 1
Synthesized PA molecules

| Molecule | Sequence | SEQ ID NO:* |
|---|---|---|
| PA1 | Palmitoyl-IAAAEEEE-NH₂ | SEQ ID NO: 1 |
| PA2 | Palmitoyl-FAAAEEEE-NH₂ | SEQ ID NO: 2 |
| PA3 | Palmitoyl-VAAAEEEE-NH₂ | SEQ ID NO: 3 |
| PA4 | Palmitoyl-YAAAEEEE-NH₂ | SEQ ID NO: 4 |
| PA5 | Palmitoyl-IAAAEEEEK(DO3A:Gd)-NH₂ | SEQ ID NO: 5 |
| PA6 | Palmitoyl-VAAAEEEEK(DO3A:Gd)-NH₂ | SEQ ID NO: 6 |

*Sequence for bolded portion
PA1 had the following structure: 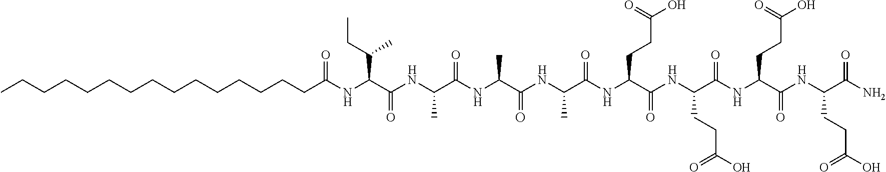 [SMILES: CCCCCCCCCCCCCCCC(=O)N[C@H](C(=O)N[C@@H](C)C(=O)N[C@@H](C)C(=O)N[C@@H](C)C(=O)N[C@@H](CCC(=O)O)C(=O)N[C@@H](CCC(=O)O)C(=O)N[C@@H](CCC(=O)O)C(=O)N[C@@H](CCC(=O)O)C(N)=O)[C@@H](C)CC]
PA5 had the following structure:

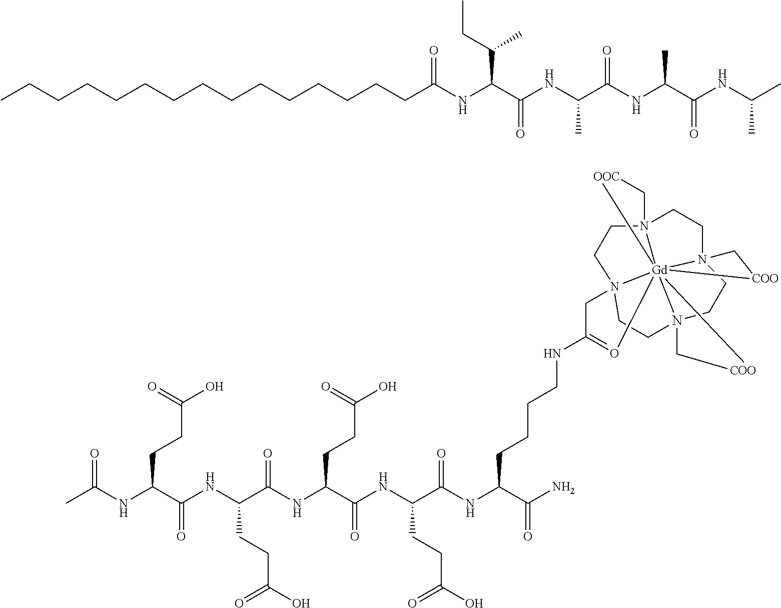

A PA design strategy was developed for tuning the pH at which the self-assembly transition into nanofibers occurs by tenths of pH units, in simulated serum salt solutions (150 mM NaCl, 2.2 mM CaCl₂) (In The Merck Manual of Diagnosis and Therapy; 19th edition ed.; Porter, R. S., Kaplan, J. L., Eds.; Merck Publishing Group: 2011). It was a goal to develop Gd³⁺-based magnetic resonance imaging agents, and 10 μM is the minimum diagnostic concentration of these agents in blood (Nunn, A. D., et al. J. Nucl. Med. 1997 41:155-62; Wedeking, P., et al. Magn. Reson. Imag. 1999 17:569-75). The PAs in this study contain a palmitic acid tail; an XAAA (SEQ ID NO:38) 3-sheet-forming region, where X is an amino acid with a nonpolar side chain; and four glutamic acid residues (Table 1). A ratio of one strongly hydrophobic amino acid (e.g., Tyrosine (Y), Valine (V), Phenylalanine (F), or Isoleucine(I)) to four glutamic acids enabled transition in the desired pH range of 6.0-6.6. PAs were synthesized by solid-phase Fmoc synthesis, and purified by reverse-phase high-performance liquid chromatography (HPLC). Their purity was assessed using analytical HPLC, electrospray ionization mass spectrometry (ESI-MS), and peptide content analysis.

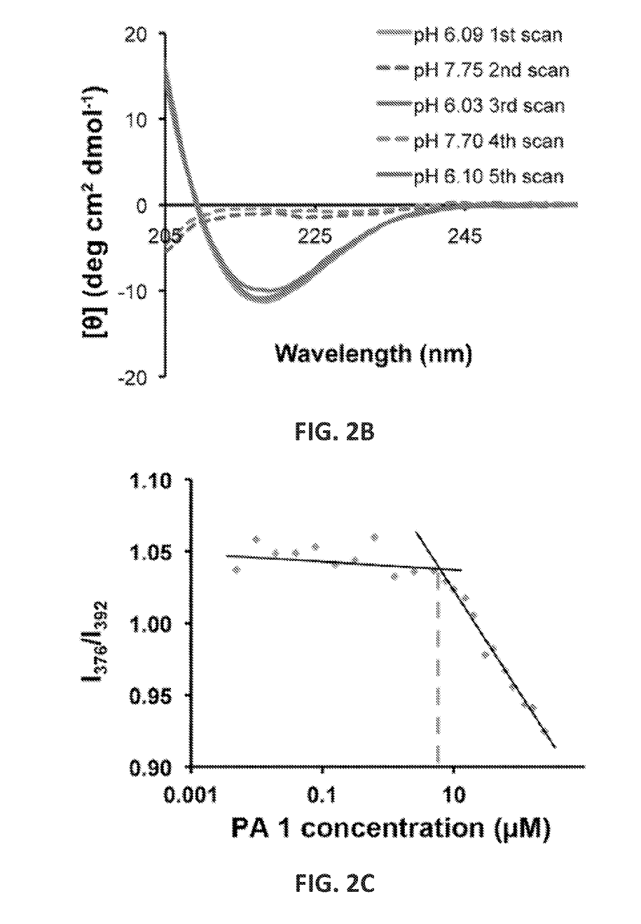

The target PA concentration (10 μM) was below the detectable limit of conventional techniques to determine the morphology such as cryoTEM and small-angle X-ray scattering. Consequently, circular dichroism (CD) spectroscopy was initially used to characterize the morphology of these PAs at various pH values. PA1 was the first molecule synthesized that underwent a self-assembly transition in the desired pH range of 6.6-7.4 at 10 μM PA concentration, in 150 mM NaCl and 2.2 mM CaCl₂ (FIG. 2A) (Goldberger, J. E., et al. Angew. Chem. Int. Ed. 2011 50:6292-95). The secondary structure exhibited a superimposable random coil morphology at pHs above 6.82. At more acidic pHs, the peptides started self-assembling into a structure with β-sheet character, which is indicative of a nanofiber morphology (Goldberger, J. E., et al. Angew. Chem. Int. Ed. 2011 50:6292-95). The transition pH from random coil to β-sheet occurred at a pH of 6.6. The transition pH was defined as the value at which the ellipticity at 205 nm rises to zero, followed by the appearance of a minimum at 218-220 nm.

TABLE 2
CD Transition pH and pKa for PAs 1-4

| Molecule | CD Transition pH | Average pKa |
|---|---|---|
| PA1 | 6.6 | 4.66 ± 0.10 |
| PA2 | 6.6 | 4.94 ± 0.09 |
| PA3 | 6.2 | 4.86 ± 0.11 |
| PA4 | 6.0 | 4.70 ± 0.10 |

For 10 μM of PAs 1-4, measured in 150 mM NaCl, and 2.2 mM CaCl₂.

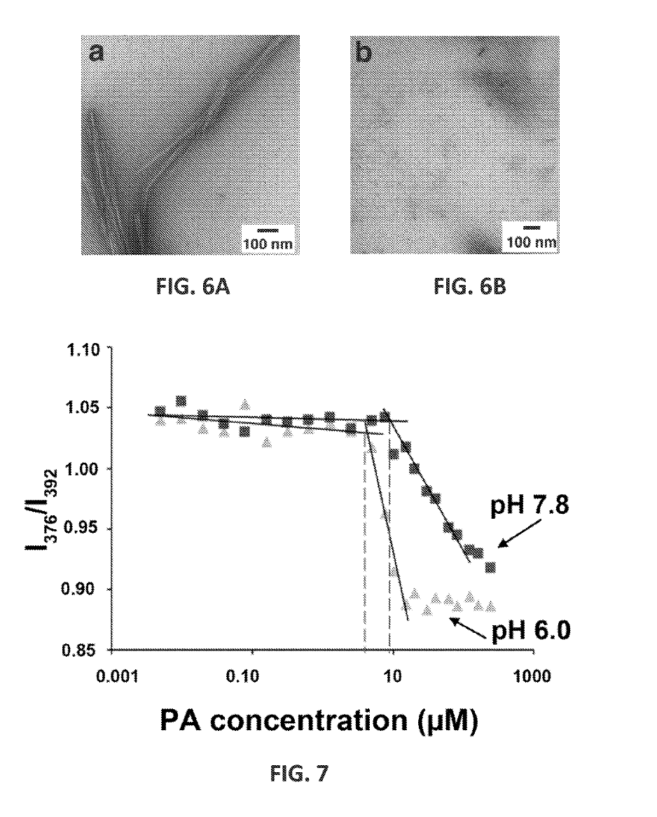

The transition between random coil and 3-sheet structure was rapid and reversible. At a pH of 7.75, HCl was added until the pH was 6.1, and the resulting n-sheet CD spectrum was collected within three minutes. An appropriate amount of NaOH was then added to reverse the pH back to 7.70, and random coil behavior was observed again. This process was repeated three times and the CD spectra were found to be superimposable with respect to pH (FIG. 2B), indicating that this self-assembly transition occurs under thermodynamic equilibrium, and requires three minutes or less to achieve the expected morphology. Conventional transmission electron microscopy (TEM) imaging was used to determine the morphology of 10 μM of PA1, at pH values of 6 and 8 (FIG. 6A, 6B). The TEM grids were prepared within three minutes of pH adjustment. At pH 6, both individual and bundled fibers were present, though much more dilute, and the isolated fibers had an average length of 590±200 nm, and an average diameter of 9.1±1.5 nm (FIG. 6A). This nanofiber diameter corresponds roughly to twice the molecular length based on MM+ molecular simulations, corresponding approximately to the expected diameter of cylindrical fibers consisting of hydrophobically collapsed β-sheets. At pH of 8, no fibers were present (FIG. 6B), confirming that the β-sheet character corresponds to the existence of fibers.

When the CD spectra show a random coil morphology, the PA molecules could either be self-assembled into spherical micelles or exist as isolated molecules in solution (Goldberger, J. E., et al. Angew. Chem. Int. Ed. 2011 50:6292-95). Because it is difficult to distinguish between staining artifacts and sample with TEM imaging at such a low concentration of sample, to determine the morphology under basic pH values, the critical aggregation concentration (CAC) was measured for PA1 at pH=6.6 using the pyrene 1:3 method (FIG. 2C) (Aguiar, J., et al. J. Colloid Interface Sci. 2003 258:116-22). The CAC was found to be 6.0 μM, which is slightly below the 10 μM concentration at which the CD spectrum was obtained. These two values are in relative agreement especially considering the arbitrary nature of defining the transition pH from the CD spectrum. Thus, the random coil behavior corresponds to isolated molecules in solution, as opposed to a spherical micellar morphology.

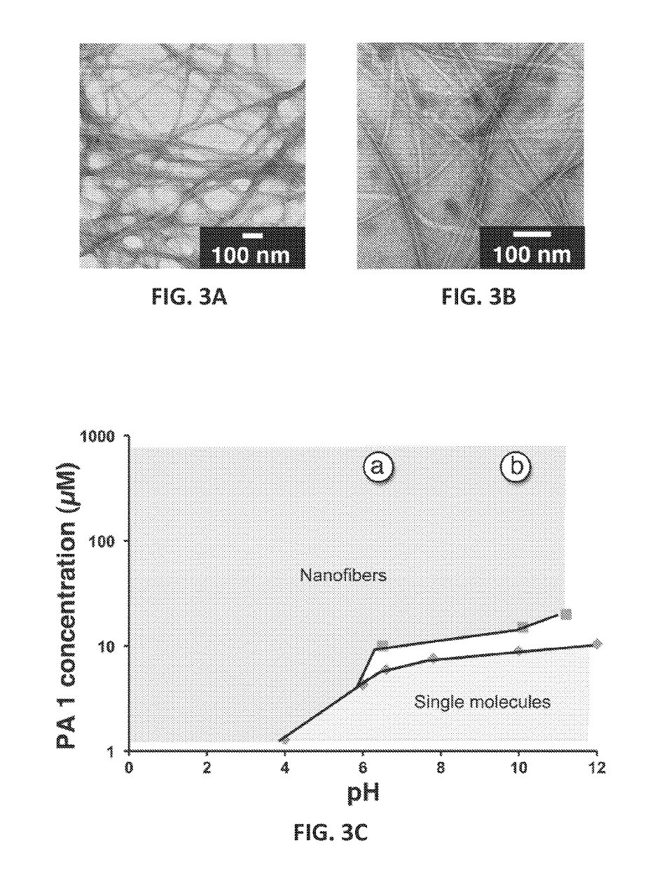

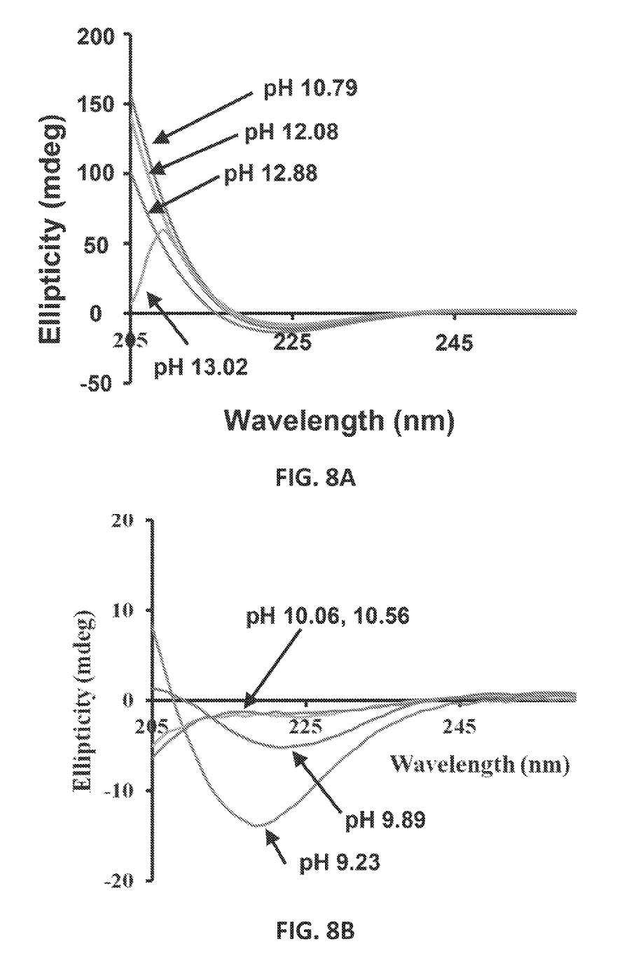

To determine the overall influence of concentration and pH on the nature of this self-assembly transition, CAC measurements were performed at pH values between 4.0 and 10.0 (FIG. 7) and CD spectra were collected at concentrations ranging from 10-30 μM (FIG. 8). The transition points determined from both techniques were plotted to generate a concentration-pH self-assembly phase diagram (FIG. 3C). PA1 exhibited both a strong concentration and pH dependence in the self-assembly transition. This concentration dependence was further confirmed via conventional transmission electron microscopy (TEM) imaging. At a pH of 6.0, and 10.0, both isolated and bundled nanofibers were observed in samples prepared at 0.5 mM concentration (FIG. 3A,3B). At pH values of 6.0 and 10.0, the isolated nanofibers had average diameters of 9.4±1.1 nm and 9.5±1.2 nm, respectively.

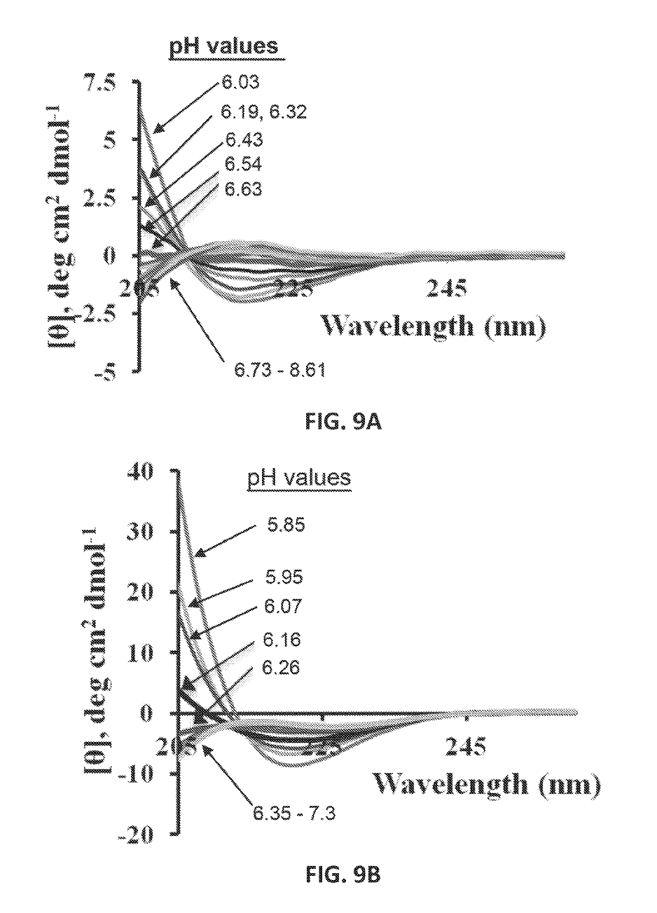

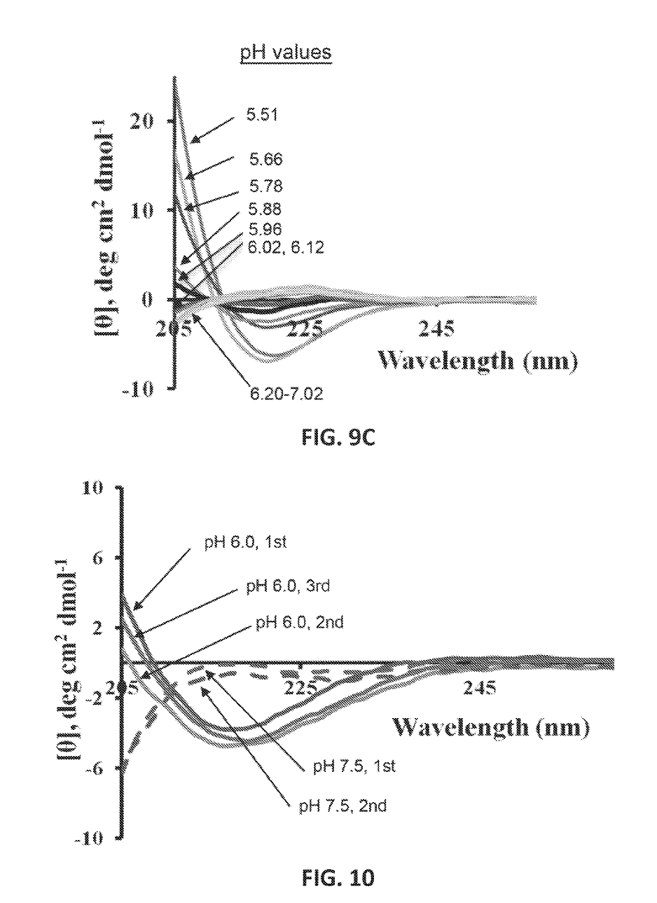

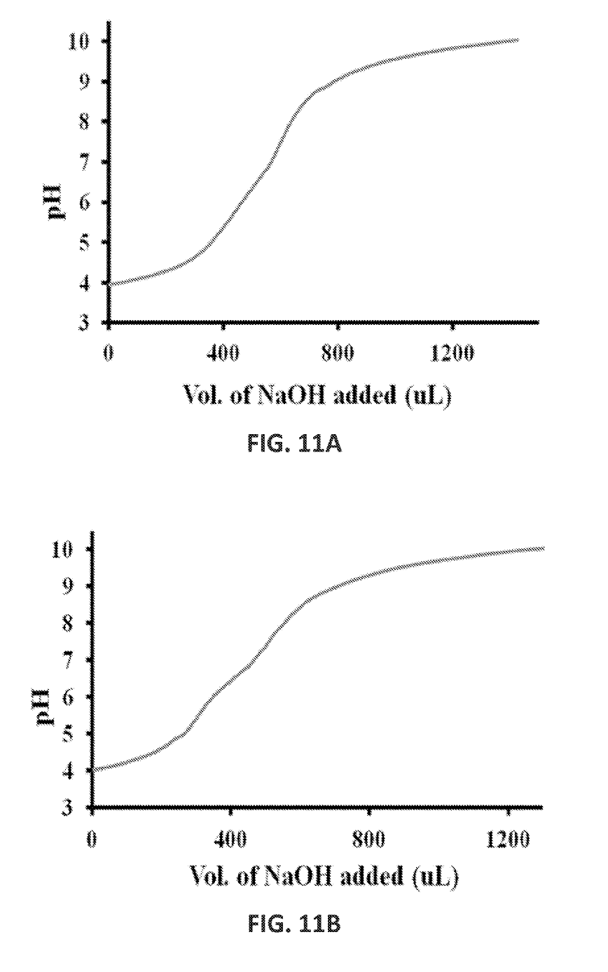

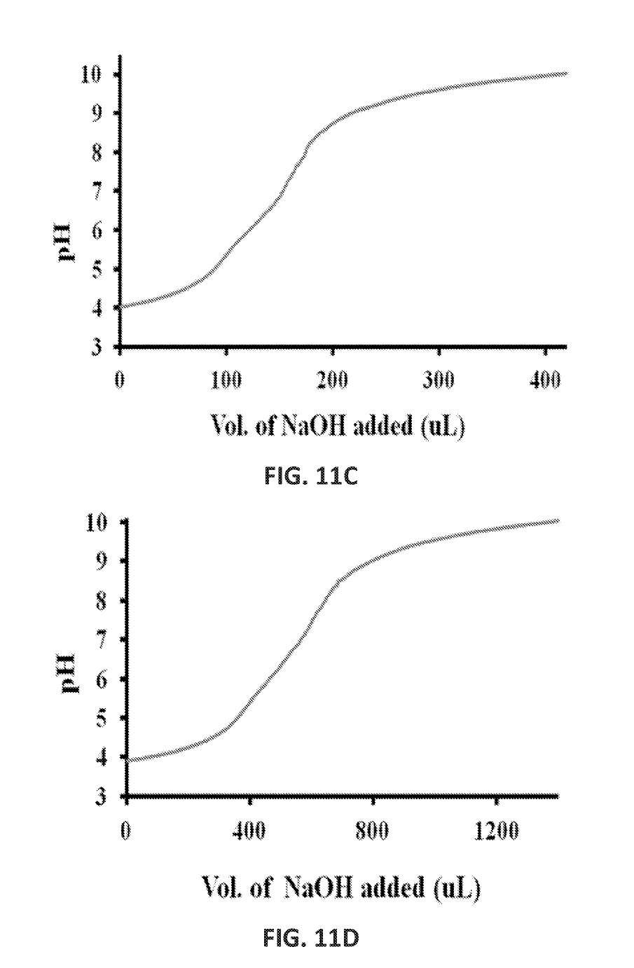

By varying the β-sheet propensity of the amino acids in the β-sheet forming region, the transition pH can be systematically tuned. In PAs 2-4, the isoleucine of PA1 was substituted with the hydrophobic amino acids phenylalanine, valine, and tyrosine. pH dependent CD spectra of PAs 2-4 at 10 μM, also showed a n-sheet to random coil transition at pH's between 6.0-6.6 (FIGS. 9A-9C). Similar to PA1, this transition was observed to be reversible (FIG. 10). Previous studies have shown that the propensity for 3-sheet formation of these amino acids follows the trend; I>F>V>Y (Kim, C. A., et al. Nature 1993 362:267-70). In PAs 1-4, the transition pH shifts to lower values with decreasing β-sheet propensity of the substituted hydrophobic amino acid (Table 2). The average pKa for the glutamic acid residues in each molecule was determined from pH titration curves (Table 2), and were found to range between 4.66-4.94 with no specific correlation with the hydrophobicity of the peptide (FIGS. 11A-11D). This indicates that the difference in self-assembly pH is not due to changes in the pKa of the glutamic acid side chains. Rather, the transition is determined by the balance between the relative attractive forces of the β-sheet forming and hydrophobic region, and the repulsive forces of the deprotonated glutamic acids in the peptide. With a stronger β-sheet forming hydrophobic segment, the transition shifts to more basic pHs. For PA1 and PA4, the transition at 10 prM occurred when 98.8% and 91%, respectively, of the glutamic acids were in the deprotonated form.

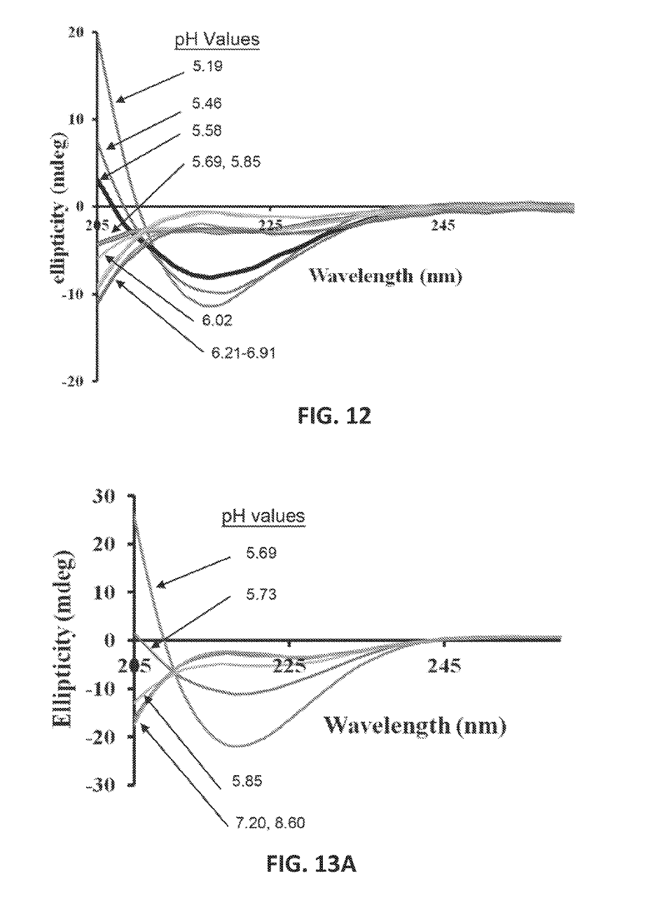

An MRI imaging moiety was then incorporated on the C-terminus of the PA. An additional lysine, conjugated to a 1,4,7-tris(carboxymethylaza)cyclododecane-10-azaacetylamide (DO3A) tag was linked to the C-terminus of PA1 and PA3 to produce PA5 and PA6. The molecule-to-nanofiber transition was still observed at 10 μM PA concentration, however the transition pH of PA5 was shifted to 5.7 (FIG. 12). Since this imaging moiety does not add excess charge, this shift towards more acidic pH wA likely due to the greater steric hindrance partly arising from the additional hydrophilicity of the DO3A restricting the formation of the self-assembled state.

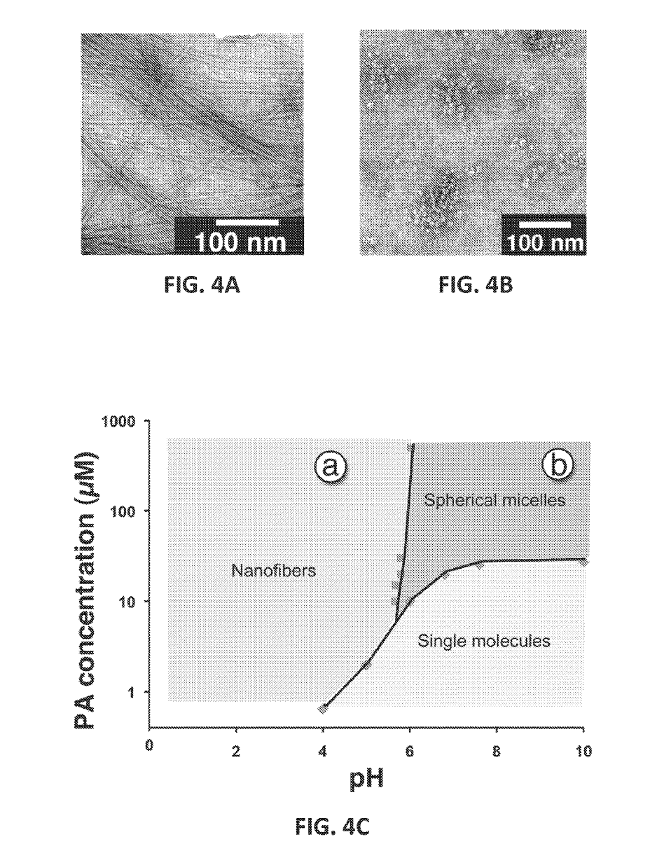

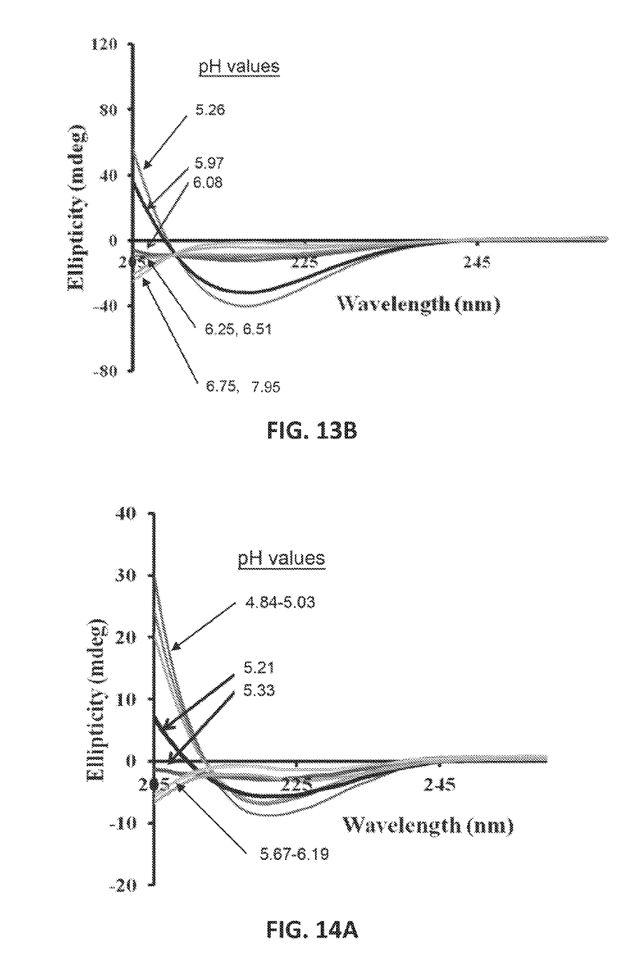

The concentration-pH self-assembly phase diagram was mapped out for PA5 (FIG. 4c). Under basic conditions and at concentrations above the CAC, a random coil secondary structure was observed in the CD spectra, which is indicative of self-assembly into a spherical micelle phase. The transition from nanofibers to spherical micelles was confirmed via TEM imaging at 0.5 mM PA at a pH of 4 and 10, respectively (FIG. 4A, 4B). The nanofibers and spherical micelles had diameters of 11.9±1.6 nm and 10.0±1.2 nm, respectively. In contrast to the nanofiber to molecule transition, the transition pH for the nanofiber to micelle transition showed relatively little concentration dependence. The nanofiber to micelle transitions at 0.5 mM and 20 μM PA, occurred at pH values of 6.0 and 5.7, respectively (FIGS. 13A, 13B). The steric bulk of the DO3A moiety increases the headgroup size of PA5 relative to PA1, thus inducing the spherical self-assembly morphology (Israelachvili, J. N. Intermolecular and Surface Forces, Second Edition; 2 ed.; Academic Press, 1992).

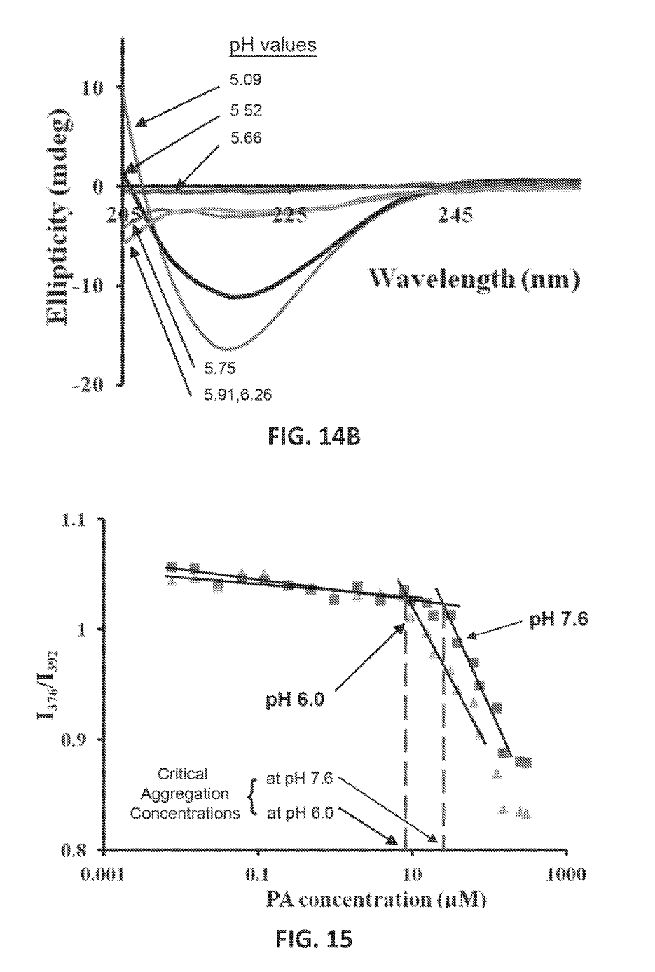

The shift in transition pH due to the change in β-sheet propensity still occurred when the Gd(DO3A) moiety was present. For each concentration, PA6 had a nanofiber to micelle transition that occurred at 0.4 units lower than PA5 (FIGS. 14A, 14B). Because the same trend occurs in these PAs irrespective of the presence of a DO3A:Gd moiety, this strategy of altering the β-sheet propensity can be generally used to systematically shift the transition pH.

Relaxivity values of water protons in the presence of PA5 at 500 μM, at pH 4 and pH 10 were found to be 8.3 and 6.6 mM⁻¹ s⁻¹, using a 1.5 T magnet. These values were higher than that which we measured for a Magnevist control standard (4.5 mM⁻¹ s⁻¹) (Stanisz, G. J., et al. Magn. Reson. Med. 2000 44:665-67; Sasaki, M., et al. Magn. Res. Med. Sci. 2005 4:145-9). This relaxivity increase from spherical micelles to nanofibers likely originates from the longer rotational correlation time when imaging agents are coupled to large molecular weight objects, which has been well-established for magnetic resonance agents coupled to polymers and peptide amphiphiles (Bull, S. R., et al. Nano Letters 2005 5:1-4; Bull, S. R., et al. Bioconjugate Chem. 2005 16:1343-48; Nicolle, G. M., et al. J. Biol. Inorg. Chem. 2002 7:757-69). The relaxivity of these systems was about 25-50% lower than other supramolecular assemblies with similar K(DO3A:Gd) linkages (Bull, S. R., et al. Nano Letters 2005 5:1-4; Accardo, A., et al. Coord. Chem. Rev. 2009, 253, 2193-213). This suggests that the Gd(DO3A) motion is independently faster than that of the nanofiber due to the conformationally flexible E₄K tether, which can be further optimized. Regardless, the primary mechanism for tumor imaging relies on the increased local concentration of the more slowly diffusing nanofibers in the tumor environment compared to the blood stream, but the improved relaxivity of fibers compared to spheres could serve as a secondary mechanism for enhanced tumor detection.

In summary, through judicious design it is possible to use the power of self-assembly to develop dynamic materials that change shape and size in response to slight changes in pH, in solutions that have monovalent and divalent ion concentrations similar to those of serum. This morphological change is rapid, reversible, and occurs under thermodynamic equilibrium, which is ideal for in vivo imaging and drug delivery applications. The molecules presented here outline a design strategy for precisely tuning self-assembly behavior.

Materials and Methods

Synthesis of Exemplary Peptide Amphiphiles (PAs):

All amino acids were purchased from AnaSpec Inc. unless otherwise specified. The peptides were synthesized by the solid-phase technique using standard Fmoc chemistry. For PAs containing lysine, Sieber resin (AAPPTEC) was used; all other peptides were synthesized using Rink Amide resin (AAPPTEC). Peptides that were made on a 0.25 mmol-scale, were synthesized on an automated peptide synthesizer (Applied Biosystems Model No. 433A) with Applied Biosystem cartridges for all but palmitic acid (Sigma Aldrich). Peptides above 0.25 mmol were synthesized manually as described in the following paragraph.

The resin was swollen in a shaker vessel with dichloromethane (DCM) for 30 minutes, the DCM was removed and dimethyl formamide (DMF) was added, followed by mechanically shaking the mixture for 30 minutes. For deprotection, 20% piperidine in DMF was used to remove the Fmoc protecting group on the resin. A Kaiser test protocol confirmed removal of the Fmoc protecting group. Coupling of the amino acid to the amine end of the resin was done through activation using either O-Benzotriazole N,N,N′,N′-tetramethyluronium hexafluorophosphate (HBTU) or 2-(7-Aza-1H-benzotriazole-1-yl)-1,1,3,3-tetramethyluronium hexafluorophosphate (HATU). The coupling solution contained 3.96 Eqv. of amino acid, 4 Eqv. of HBTU/HATU, 4 Eqv. of N-Hydroxybenzotriazole (HOBt) or 1-Hydroxy-7-azabenzotriazole (HOAt), and 8 Eqv. of Diisopropyl ethylamine (DIPEA) with respect to peptide allowing at least 3 hours of coupling per amino acid. The surfactant Triton X-100 was added to the coupling solution and to the latter amino acids to aid in coupling efficiency. Resin cleavage of the peptide was done by addition of the following solutions: For the Rink Amide resin, a solution of 95% Trifluoroacetic acid (TFA), 2% Anisole, 2% water was used and for Sieber Resin cleavage, a solution of 1% TFA, 2% Anisole, 1% Triisopropyl silane (TIS) and 96% DCM was used; shaken for at least 2 hours. The TFA was removed under vacuo and the PA was precipitated using two 20 mL portions of cold diethyl ether. The crude peptide was filtered and washed with cold diethyl ether.

Purification of Peptide Amphiphiles:

The crude peptide amphiphile was dissolved in 0.1% NH₄OH solution at approximately 10 mg/mL concentration by vigorously shaking and sonicating until the solution turned clear. To aid in dissolution, an additional drop of concentrated NH₄OH was added to the solution. The PA solution was filtered first using a 0.45 μm syringe filter (Whatman), followed by filtration through a 0.2 μm syringe filter. The sample was then purified on a Shimadzu preparative HPLC system (dual pump system controlled by LC-MS solution software) with an Agilent PLRP-S polymer column (Model No. PL1212-3100 150 mm×25 mm) under basic conditions. The product was eluted with a linear gradient of 10% Acetonitrile to 100% Acetonitrile over 30 minutes containing 0.1% NH₄OH (v/v). The purity of the collected fractions was verified using an electrospray ionization time-of-flight mass spectrometer (Bruker) and a Shimadzu analytical HPLC system. Fractions greater than 90% purity were combined; the Acetonitrile (MeCN) was removed by vacuum before freeze-drying.

Synthesis and Purification of Protected Tri-Tert-Butyl Ester DO3A Derivative:

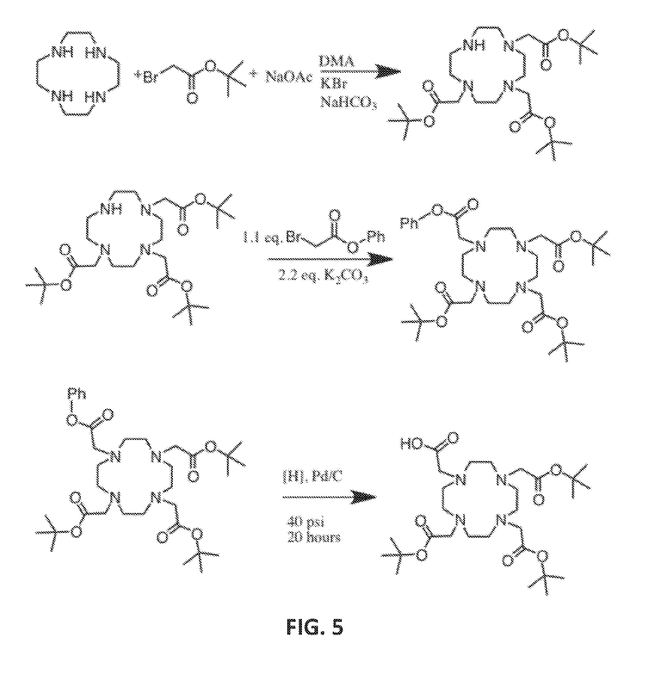

The synthesis scheme for a protected tri-tert-butyl ester DO3A derivative is shown in FIG. 5. All reagents were purchased from Sigma Aldrich and used without further purification unless specified. 30 g of cyclen (1,4,7,10-tetraazacyclododecane) was combined with 42.9 g of powdered, dry sodium acetate in 400 mL of N,N-dimethylacetamide in a round bottom flask and stirred for 30 minutes with an overhead glass rod stirrer. The round bottom flask containing the slurry was placed in an ice bath until the temperature reached 0° C. 77.1 mL of tert-butyl bromoacetate was dissolved in 150 mL of N, N,-dimethylacetamide and added drop-wise to the slurry at 0° C. over a period of 25 minutes. The slurry was equilibrated back to room temperature and stirred for 5 days. A separate solution was prepared by dissolving 30 g of potassium bromide (KBr) in 2 L of deionized water (Millipore) followed by stirring and heating to a temperature of 50° C. After the KBr solution reached 50° C., it was added to the slurry forming a yellow colored solution. The pH was adjusted to 9.0 by the addition of powdered sodium bicarbonate (checked via litmus paper) when the desired product, acetic acid tert-butyl ester hydrobromide (4,7-bis-tert-butoxycarbonylmethyl-1,4,7,10-tetraaza-cyclododec-1-yl) precipitated. It was allowed to settle for 4 hours without stirring, followed by vacuum filtering and drying, yielding a white powder. 10.0 g of this acetic acid tert-butyl ester hydrobromide was dissolved in 50 mL of MeCN and combined with 5.1077 g (2.2 eq.) of finely powdered, dry potassium carbonate and stirred for 30 minutes. 2.927 mL of Benzyl bromo acetate (1.1 eq.) was added drop-wise to the solution and stirred overnight at 50° C.-60° C. The mixture was then cooled to room temperature and vacuum filtered, with the desired product in the filtrate. The solid was washed twice with MeCN. The combined MeCN was removed by evaporation under vacuum, yielding the tri-tert-butyl ester form of DO3A, a viscous yellow gel. NMR and ESI-MS confirmed the presence of the desired product. The crude product was purified using flash-column chromatography using 50.0 g of silica gel for every 1.0 g of tri-tert-butyl ester form of DO3A using DCM as the mobile phase. The product was eluted from the column using a gradient elution, starting with 2% MeOH in DCM to 6% of MeOH in DCM. The elution of the desired product was followed by Thin Layer Chromatography, using 10% MeOH in DCM as the mobile phase. Pure fractions were combined and the solvents evaporated under vacuum. The residue was then dissolved in approximately 50 mL of MeOH in deionized water (Millipore) at a ratio of 9:1. Palladium on carbon catalyst was added to the solution in 20% by weight with respect to tri-tert-butyl ester form of DO3A. The sample was hydrogenated under 50-psi hydrogen pressure overnight followed by filtration of the solid catalyst. The filtrate containing DO3A was evaporated under vacuum to remove the methanol then 100 mL of deionized water was added to the solution. Diethyl ether (50 mL) was added 3 times to the solution in a separatory funnel to extract the non-hydrogenated product. Solvent was removed by evaporation and the solution was freeze-dried to remove remaining deionized water, yielding a yellowish powder. NMR spectroscopy and ESI-MS were used to confirm the presence of DO3A and check purity.

Attachment of DO3A to Peptide Amphiphiles:

To attach the DO3A derivative, an additional Lysine (K) with its side chain amine protected by a methyl trityl group, was coupled to the PA sequence after the last glutamic acid (E). The cleavage cocktail used to cleave the PA from the Sieber resin also removed the methyl trityl group from the lysine. The DO3A was then coupled to the side chain amine group of the lysine in solution phase using the coupling solution mentioned earlier (synthesis of peptide amphiphiles) with the exception of 1 eqv. of the tri-tert-butyl ester DO3A derivative.

Incorporation of Gd³⁺ in the PA-DO3A Conjugates:

2.27 mg of the previously prepared PA-DO3A conjugate was dissolved in 1.0 mL of water and combined with 2 eq. of GdCl₃ in 0.01 M HCl. The reaction was set to stir in an oil bath at 60° C. for 30 min. The pH of the solution was gradually adjusted from approximately pH 2 to pH 4-5 using small amounts of 0.050 M NaOH. The resultant solution was stirred for 24 hours at 60° C. A small sample was removed and analyzed by MALDI-MS to determine the extent of reaction completion. The pH was then raised to 8-9 over a period of an hour using ammonium hydroxide (to precipitate excess Gd³⁺ as Gd(OH)₃) followed by the addition of EDTA (to chelate excess free Gd³⁺) and filtered using a 0.2 μm syringe filter. The solution was dialyzed against Millipore water to remove NaCl, free Gd³⁺, and EDTA-Gd³⁺. The buffer water for dialysis was changed 4 times over a period of 24 hours. The PA-DOTA-Gd³⁺ solution was finally freeze-dried to recover a white fluffy powder.

Peptide Content Analysis:

Peptide content analysis was performed on lyophilized samples to verify the amino acid stoichiometry and determine the residual salt concentration for PA1-5. The relative residue stoichiometry was within ±5% of the expected values for all amino acids in PA1-5. The mg of total peptide amphiphile/mg of solid is listed in Table 3 below. All further CAC and CD measurements were scaled by these factors to determine the true concentration.

TABLE 3
Peptide Content

| PA | mg PA/mg Solid |
|---|---|
| PA1 | 88% |
| PA2 | 73% |
| PA3 | 91% |
| PA4 | 61% |
| PA5 | 96% |

Circular Dichroism (CD) Spectroscopy:

Measurements were done on a Jasco-815 Circular Dichroism spectrometer using 1 cm path length quartz cuvettes. 10 μM solutions of peptide amphiphiles were prepared in 150 mM NaCl and 2.2 mM CaCl₂ by dilution from a concentrated PA stock (0.5-1 mM, pH 9). Double de-ionized Milli-Q water was used for preparing all solutions. The solutions were then heated at 80° C. for 30 minutes in a water bath and gradually cooled to room temperature. An Accumet XL15 pH meter (Fisher Scientific) coupled with an Orion Ross Ultra semi-micro electrode (8103BNUWP, Thermo Scientific) was used to adjust the pH of the solution to the desired value followed by collection of the CD spectra Each trace shown was averaged over 3 accumulations and was baseline subtracted using aqueous solutions containing salts only. All spectra are cut-off below 205 nm due to absorption by NaCl and CaCl₂.

Transmission Electron Microscopy (TEM):

TEM images were obtained using solutions of either 10 μM or 0.5 mM peptide amphiphile concentration, as well as 150 mM NaCl and 2.2 mM CaCl₂ in Milli-Q water. The solutions were first heated at 80° C. for 30 minutes in a water bath and then gradually cooled to room temperature. This was followed by pH adjustment using either HCl or NaOH. 5 μL of this solution was pipetted onto a Carbon Formvar grid (Electron Microscopy Sciences) and allowed to sit for 2 minutes before being wicked dry using filter paper. For the 10 μM experiments that were performed to determine the time dependence of nanofiber formation, solutions were dropcast onto the grid within three minutes of pH adjustment. The samples were then negatively stained using 1 wt % uranyl acetate and imaged under a FEI Tecnai G2 Bio TWIN TEM system, operating at 100 kV. All TEM experiments were performed in triplicate, using at least three different freshly prepared solutions and grids.

pH Titration of Peptide Amphiphiles:

The titration measurements were conducted on 10 μM peptide amphiphile solutions prepared in 150 mM NaCl and 2.2 mM CaCl₂ using milli-Q water. The solution was heated at 80° C. for 30 minutes followed by slow cooling at room temperature. The pH of the solution was then adjusted to 4 using HCl. Finally, an Accumet XL15 pH meter (Fisher Scientific) coupled with an Orion Ross Ultra semi-micro electrode (8103BNUWP, Thermo Scientific) was used to track changes in pH of the solution as NaOH solution was added in small increments. pKa values were obtained from the second inflection points of the first derivative plots of the titration data. The first transition corresponds to neutralization of excess HCl. The calculated pKas reflect the average pKa for all four glutamic acids.

Critical Aggregation Concentration (CAC) Determination Using the Pyrene 1:3 Method:

For the CAC measurements, a series of solutions of the PA with concentrations ranging from 10 nM to 300 μM were prepared using serial dilutions in 150 mM NaCl and 2.2 mM CaCl₂. The final concentration of pyrene in each solution was fixed to be 1.23 μM. This was followed by pH adjustment of the solutions using careful additions of HCl or NaOH. 70 μL of each solution was transferred to a 96-well plate and the fluorescence emission of pyrene was monitored using a hybrid reader fluorimeter (BioTek Synergy H4) at room temperature. The excitation wavelength was set at 335 nm. The ratio of the intensities of emissions at 376 nm and 392 nm were then plotted as a function of the PA concentration (log scale). The CAC was determined from an abrupt change in the slope of the plot using the least-squares fitting technique.

Molecular Simulations:

The molecular length was estimated through models derived from the MM+ geometry optimization as implemented using the Hyperchem Software Suite. The molecule length was derived from the energy-minimized geometry of the fully extended molecule. The value for molecular length was assumed to be the distance between the final C atom on the alkyl chain and the end amide C atom on the terminal glutamic acid (for PAs 1-4).

Magnetic Resonance Imaging (MRI) Measurements:

An MR relaxometry phantom was built by fixing 5 mm NMR sample tubes containing PA samples at a concentration of 500 μM and pH values 4 and 10 in a 600 ml beaker filled with deionized water. The samples also contained 150 mM NaCl and 2.2 mM CaCl₂. The phantom was scanned on a 1.5 Tesla Signa Excite MRI scanner using an 8-channel phased-array head coil (GE Healthcare, Milwaukee, Wis., USA). R relaxometry data were acquired using an Inversion Recovery Fast Spin-Echo (IR-FSE) sequence with the following parameters: inversion time TI=50/100/200/400/600/800/1200/1600/3200 ms; repetition time TR=8000 ms; echo time TE=11 ms; echo train length=10; 90° flip angle; 100×100 mm² field-of-view (FOV); 0.5 mm in-plane resolution; 2 mm slice thickness; single coronal slice placed at the center of the phantom. Sample longitudinal relaxation rates (R1) were calculated by fitting the MR signal intensities observed at different TIs (S(TI)) to a three parameter model [Lu et al., MRM 2004]:

S(TI)=|S₀×(1−C×exp(−TI×R1))|

Where S₀ is the equilibrium signal, C is a constant accounting for imperfect inversion of magnetization. The r1 relaxivity was calculated as the slope in the linear relationship between sample concentrations and their R1 relaxation rates.

### Example 2: Designing Peptide Amphiphiles that Transition from Spherical Micelles in Serum to Cylindrical Fibers in Low pH Tumor Tissue

Pan-cancer biocompatible diagnostic (or theranostic) imaging agents or therapeutic agents that circulate through the bloodstream as isolated molecules or self assembled micelles of hydrodynamic diameter >10 nm that spontaneously and reversibly transform into long cylindrical nanofibers >100 nm only when encountering the extracellular acidic (pH 6.4-7.3) tumor microvasculature were designed. Because of the significantly slower diffusion constant of cylindrical nanofibers >1000 nm in length, the imaging agent is expected to significantly accumulate in the acidic tumor, which continuously resupplies its microenvironment with protons.

Peptide amphiphiles (PAs) were designed to contain a particular sequence of amino acids, lipids, a DO3A agent designed to bind to trivalent metal ions such as Gd³⁺ (for MRI), Lu³⁺ (for 177Lu radiotherapy), Tb³⁺ (for fluorescent analysis), and Ga³⁺ or In³⁺ (for ⁶⁸Ga PET/CT or PET/MRI or ¹¹¹In SPECT/CT), and with or without an ethylene glycol shell, that can undergo this transformation in a simulated blood environment (150 mM NaCl, 2.2 mM CaCl₂). The pH of this transition at any particular concentration can occur between 5.1 and 7.3.

Numerous peptide amphiphiles have been synthesized and studied in the past; however these systems completely and irreversibly assemble into nanofibers that have with lengths that are >100 nm. Here, PAs were designed in such a way that the attractive supramolecular forces (hydrophobic-hydrophobic interactions, β-sheet formation) and the repulsive supramolecular forces (electrostatic repulsion, sterics) of the molecule are precisely balanced. The repulsive forces can be increased by increasing the number of charged amino acid residues, or adding a unit with larger hydrophilicity or greater steric hindrance such as a K(DO3A)². Increasing the attractive forces can be done by using longer alkyl chains, as well as increasing the number of β-sheet forming residues Indeed, the pH at which a molecule undergoes this transition depends on the relative ratio of the standard peptide amphiphile molecule, which can contain the following components;

(Cₙ)—(BₒUₚ)-(Neg_{q}K(DO3A:M³⁺))-(PEG)ᵣ-propionic amide;

(Cₙ)—(BₒUₚ)-(Neg_{q-1}K(DO3A:M³⁺))-(PEG)ᵣ-propionic acid;

(Cₙ)—(BₒUₚ)-(Neg_{q}K(DO3A:M³⁺))—NH₂; or

(Cₙ)—(BₒUₚ)-(Neg_{q-1}K(DO3A:M³⁺))—COOH;

where Cₙ=lipid tail with n carbons such as C₁₆=palmitoyl, C₁₄=myristoyl (n=12-18).

In the β-sheet forming region

B^{s}=an amino acid with high β-sheet propensity (o=1 or 2)

U=an uncharged amino acid that with poor β-sheet propensity

Note that the order of B^{s} to U does not strongly affect the transition.

In the Charged Region

Neg=an anionic amino acid (q=3-7)

K(DO3A:M³⁺)=a lysine with a conjugated to a 1,4,7-tris(carboxymethylaza)cyclododecane-10-azaacetylamide tag M³⁺=Gd³⁺ for MRI

The order of Neg to K(DO3A:M³⁺) does not strongly affect the transition.

Also, the order of B^{s}:U:Neg:K(DO3A:M³⁺) does not strongly affect the transition

To Minimize Immune Response

PEG=ethylene glycol (r=0-8); (also branched PEG)

The amino acids are classified into Table 4. The chirality of the amino acids can be either d-, or l- with minimal change of properties

TABLE 4
Natural Amino Acids

Strongly β-sheet forming amino acids (B^{s})
Isoleucine (I)
Phenylalanine (F)
Valine (V)
Tyrosine (Y)
Amino Acid with Intermediate Beta-sheet Propensity (Bⁱ)
Methionine (M)
Leucine (L)
Threonine (T)
Glutamine (Q)
Tryptophan (W)
Asparagine (N)
Uncharged amino acid with poor β-sheet propensity (U)
Serine (S)
Alanine (A)
Glycine (G)
Anionic Amino acids (Neg)
Aspartic Acid (D)
Glutamic Acid (E)

For example, the peptide amphiphile molecule can be palmitoyl-VAAAEEEEK(DO3A:Gd)-PEG-propionic amide (SEQ ID NO:6 for bold portion) (PA64), which has the following structure:

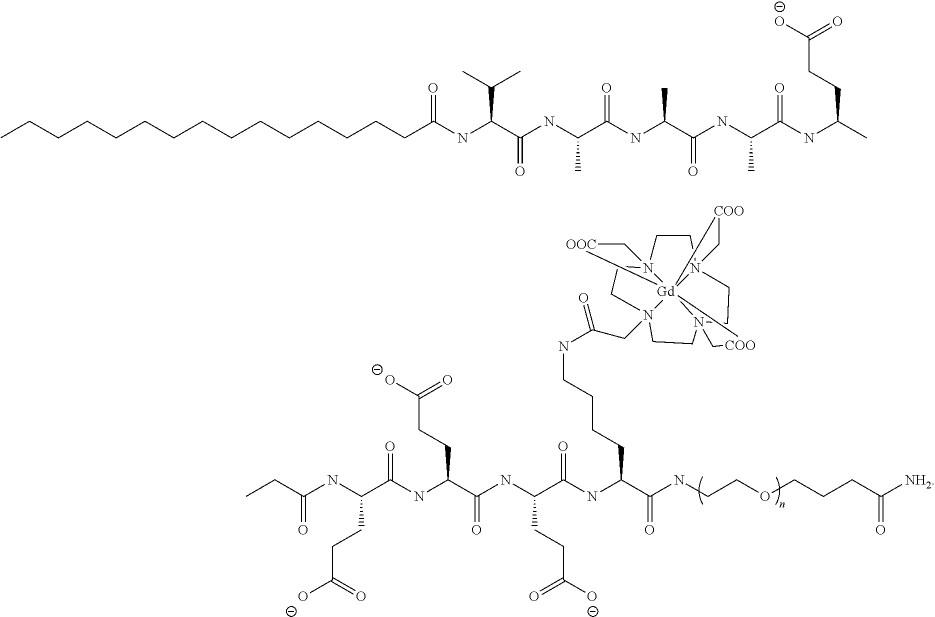

The most important factor for enabling this transition in a simulated serum environment lies with optimizing the n:o:q ratio.

n:o:q=16-17:1:3-4 with Amide Termination

In some cases, PAs having a C₁₆ (palmitoyl) or a C₁₇ (heptadecanoyl) chain (n=16-17) and one B^{s} amino acid (o=1), need 3-4 N amino acids (q=3-4). Also this transition can occur in the presence or absence of an ethyleneglycol-propionic amide tail with r=0-6.

The following PAs were synthesized and characterized for phase transition in simulated serum environment (150 mM NaC, 2.2 μM CaCl₂)

TABLE 5
PAs where n:o:q = 16-17:1:3-4 with amide termination that have a
basic to acidic transition from spherical micelle to cylindrical fiber

| | | | pH transition |
|---|---|---|---|
| | | | point at |
| Molecule | Peptide Sequence | SEQ ID NO:* | 10 μm PA |
| PA5 | Palmitoyl-IAAAEEEEK(DO3A:Gd)-NH₂ | SEQ ID NO: 5 | 5.7 |
| PA7 | Palmitoyl-IAAAEEEEK(DO3A:Tb)-NH₂ | SEQ ID NO: 5 | 5.7 |
| PA8 | Palmitoyl-IAAAEEEEK(DO3A:Lu)-NH₂ | SEQ ID NO: 5 | 5.7 |
| PA6 | Palmitoyl-VAAAEEEEK(DO3A:Gd)-NH₂ | SEQ ID NO: 6 | 5.3 |
| PA9 | Palmitoyl-YAAAEEEEK(DO3A:Gd)-NH₂ | SEQ ID NO: 7 | 5.1 |

*Sequence for bolded portion

The Phase diagrams were determined using a combination of circular dichroism measurements and critical micelle measurements as shown below.

In these above PA sequences the most critical factor for fine-tuning the transition lies with the relative β-sheet propensity. By varying B^{s} with β-sheet propensity the transition pH can be fine-tuned.

TABLE 6
PAs where n:o:q = 16-17:1:3-4 with amide termination that have
a basic to acidic transition from small molecule to cylindrical
nanofiber

| | | | pH transition |
|---|---|---|---|
| | | | point at |
| Molecule | Peptide Sequence | SEQ ID NO:* | 10 μm PA |
| PA1 | Palmitoyl-IAAAEEEE-NH₂ | SEQ ID NO: 1 | 6.6 |
| PA10 | Palmitoyl-FAAAEEEE-NH₂ | SEQ ID NO: 2 | 6.6 |
| PA11 | Palmitoyl-VAAAEEKE-NH₂ | SEQ ID NO: 3 | 6.2 |
| PA12 | Palmitoyl-YAAAEEEE-NH₂ | SEQ ID NO: 4 | 6.0 |

*Sequence for bolded portion

The order of B to U and number of U amino acids is not expected to strongly affect the pH and concentration dependent self-assembly properties. The order of Neg to K(DO3A:M³⁺) is also not expected to strongly affect the pH and concentration dependent self-assembly properties.

TABLE 7
PAs sequences where n:o:q = 16-17:1:3-4 with amide termination

| Molecule | Peptide Sequence | SEQ ID NO:* |
|---|---|---|
| PA13 | Palmitoyl-AYAAEEEEK(DO3A:Gd)-NH₂ | SEQ ID NO: 8 |
| PA14 | Palmitoyl-AAYAEEEEK(DO3A:Gd)-NH₂ | SEQ ID NO: 9 |
| PA15 | Palmitoyl-AAAYEEEEK(DO3A:Gd)-NH₂ | SEQ ID NO: 10 |
| PA16 | Palmitoyl-YAAAEEEK(DO3A:Gd)-NH₂ | SEQ ID NO: 11 |
| PA17 | Palmitoyl-IAAAK(DO3A:Gd)EEEE-NH₂ | SEQ ID NO: 12 |
| PA18 | Palmitoyl-IAAAEEK(DO3A:Gd)EE-NH₂ | SEQ ID NO: 13 |
| PA19 | Palmitoyl-IEEEEK(DO3A:Gd)-NH₂ | SEQ ID NO: 14 |
| PA20 | Palmitoyl-IAEEEEK(DO3A:Gd)-NH₂ | SEQ ID NO: 15 |
| PA21 | Palmitoyl-IAAAEEEK(DO3A:Gd)-NH₂ | SEQ ID NO: 16 |
| PA22 | Palmitoyl-IAAAEEEEK(DO3A:Gd)-PEG-propionic amide | SEQ ID NO: 5 |
| PA23 | Palmitoyl-IAAAEEEEK(DO3A:Gd)-(PEG)₂-propionic amide | SEQ ID NO: 5 |
| PA24 | Palmitoyl-IAAAEEEEK(DO3A:Gd)-(PEG)₃-propionic amide | SEQ ID NO: 5 |
| PA25 | Heptadecanoyl-IAAAEEEEK(DO3A:Gd)-NH₂ | SEQ ID NO: 5 |

*Sequence for bolded portion

n:o:q=16-17:1:2-3 with Carboxylic Acid Termination

PAs having a C₁₆ (palmitoyl) or a C₁₇ (heptadecanoyl) chain (n=16-17) and one B^{s} amino acid (n=1), but have an anionic carboxylic acid termination at the C-terminus need only 2-3 Neg amino acids (q=2-3). Also this transition can occur in the presence or absence of an ethyleneglycol-propionic acid tail with r=0-6. Example PAs that are expected to undergo this transition are shown in Table 8.

TABLE 8
PA sequences where n:o:q = 16-17:1:2-3 with carboxylic acid termination
that are expected to have a basic to acidic transition from spherical
micelle to cylindrical fiber

| Molecule | Peptide Sequence | SEQ ID NO:* |
|---|---|---|
| PA26 | Palmitoyl-AYAAEEEK(DO3A:Gd)-COOH | SEQ ID NO: 17 |
| PA27 | Palmitoyl-AAYAEEEK(DO3A:Gd)-COOH | SEQ ID NO: 18 |
| PA28 | Palmitoyl-AAAYEEEK(DO3A:Gd)-COOH | SEQ ID NO: 19 |
| PA29 | Palmitoyl-YAAAEEEK(DO3A:Gd)-COOH | SEQ ID NO: 11 |
| PA30 | Palmitoyl-IAAAK(DO3A:Gd)EEE-COOH | SEQ ID NO: 20 |
| PA31 | Palmitoyl-IAAAEEK(DO3A:Gd)E-COOH | SEQ ID NO: 21 |
| PA32 | Palmitoyl-IEEEK(DO3A:Gd)-COOH | SEQ ID NO: 22 |
| PA33 | Palmitoyl-IAEEEK(DO3A:Gd)-COOH | SEQ ID NO: 23 |
| PA34 | Palmitoyl-IAAAEEK(DO3A:Gd)-COOH | SEQ ID NO: 24 |
| PA35 | Palmitoyl-IAAAEEEK(DO3A:Gd)-PEG-propionic acid | SEQ ID NO: 16 |
| PA36 | Palmitoyl-IAAAEEEK(DO3A:Gd)-(PEG)₂-propionic acid | SEQ ID NO: 16 |
| PA37 | Palmitoyl-IAAAEEEK(DO3A:Gd)-(PEG)₃-propionic acid | SEQ ID NO: 16 |
| PA38 | Heptadecanoyl-IAAAFEEK(DO3A:Gd)-COOH | SEQ ID NO: 16 |

*Sequence for bolded portion

n:o:q=15-16:2:5-7 with Amide Termination

With two strongly β-sheet forming hydrophobic amino acids (o=2) and either a C₁₅ (pentadecanoyl) or a C₁₆ (palmitoyl) (n=15-16) the range of possible molecules that can undergo this transition in serum require 5-7 charged Neg residues (q=5-7) in the presence or absence of an ethyleneglycol-propionic amide tail with r=0-6. Example PAs that are expected to undergo this transition are shown in Table 9.

TABLE 9
PA sequences where n:o:q = 15-16:2:5-7 with amide termination that are
expected to have a basic to acidic transition from spherical micelle to
cylindrical fiber

| Molecule | Peptide Sequenc | SEQ ID NO:* |
|---|---|---|
| PA39 | Pentadecanoyl-IAAAEEEEK(DO3A:Gd)-NH₂ | SEQ ID NO: 5 |
| PA40 | Palmitoyl-VVAAEEEEE-K(DO3A:Gd)-NH₂ | SEQ ID NO: 25 |
| PA41 | Palmitoyl-YYAAEEEEE-K(DO3A:Gd)-NH₂ | SEQ ID NO: 26 |
| PA42 | Palmitoyl-YYAAEEEEEE-K(DO3A:Gd)-NH2 | SEQ ID NO: 27 |
| PA43 | Palmitoyl-YYAAEEEEEEE-K(DO3A:Gd)-NH₂ | SEQ ID NO: 28 |
| PA44 | Palmitoyl-YYAAAEEEEK(DO3A:Gd)-PEG-propionic amide | SEQ ID NO: 29 |
| PA45 | Palmitoyl-YYAAAEEEEK(DO3A:Gd)-(PEG)₂-propionic amide | SEQ ID NO: 29 |
| PA46 | Palmitoyl-YYAAAEEEEK(DO3A:Gd)-(PEG)₃-propionic amide | SEQ ID NO: 29 |

*Sequence for bolded portion

In contrast, numerous molecules were synthesized with two strongly hydrophobic amino acids that self-assemble as cylindrical nanofibers across all pH values in simulated serum solutions. In these sequences, the attractive supramolecular forces are too strong (Table 10).

TABLE 10
PAs with amide termination that self-assemble as cylindrical
nanofibers across all pH values (10 μM, pH 5-10)

| Molecule | Peptide Sequence | |
|---|---|---|
| PA47 | Palmitoyl-VVAAEEEE-NH₂ | SEQ ID NO: 30 |
| PA48 | Palmitoyl-AAVVEEEE-NH₂ | SEQ ID NO: 31 |
| PA49 | Palmitoyl-AAYYEEEE-NH₂ | SEQ ID NO: 32 |
| PA50 | Palmitoyl-AAIIEEEE-NH₂ | SEQ ID NO: 33 |
| PA51 | Palmitoyl-YYYYEEEE-NH₂ | SEQ ID NO: 34 |
| PA52 | Palmitoyl-IAAAEEEEYK(DO3A:Gd)-NH₂ | SEQ ID NO: 35 |

*Sequence for bolded portion

Alternatively, in the sequences below, when the attractive forces are too weak either due to the shorter alkyl chain, or the weakness of these molecules assemble as single molecules at all pH values (Table 11).

TABLE 11
PAs with amide termination that remain single
molecules at all pH values (10 μM, pH 5-10)

| Molecule | Peptide Sequence | SEQ ID NO:* |
|---|---|---|
| PA53 | Palmitoyl-TTTEEEE-NH₂ | SEQ ID NO: 36 |
| PA54 | Myristol-AAYYEEEE-NH₂ | SEQ ID NO: 32 |
| PA55 | Lauryl-AAYYEEEE-NH₂ | SEQ ID NO: 32 |
| PA56 | Palmitoyl-AATTEEEE-NH₂ | SEQ ID NO: 37 |

*Sequence for bolded portion

n:o:q=15-16:2:4-6 with Acid Termination

With two strongly β-sheet forming hydrophobic amino acids (o=2), acid termination, and either a C₁₅ (pentadecanoyl) or a C₁₆ (palmitoyl) (n=15-16), the range of possible molecules that can undergo this transition in serum requires 4-6 charged Neg residues (q=4-6) in the presence or absence of an ethyleneglycol-propionic acid tail with r=0-6. Example PAs that are expected to undergo this transition in serum are shown in Table 12.

TABLE 12
PA sequences where n:o:q = 15-16:2:4-6 with acid termination that are
expected to have a basic to acidic transition from spherical micelle to
cylindrical fiber

| Molecule | Peptide Sequence | SEQ ID NO:* |
|---|---|---|
| PA57 | Pentadecanoyl-IAAAEEEEK(DO3A:Gd)-COOH | SEQ ID NO: 5 |
| PA58 | Palmitoyl-YYAAEEEE-K(DO3A:Gd)-COOH | SEQ ID NO: 26 |
| PA59 | Palmitoyl-YYAAEEEEEE-K(DO3A:Gd)-COOH | SEQ ID NO: 27 |
| PA60 | Palmitoyl-YNAAEEEEEE-K(DO3A:Gd)-COOH | SEQ ID NO: 27 |
| PA61 | Palmitoyl-IAAAEEEEK(DO3A:Gd)-PEG-propionic acid | SEQ ID NO: 5 |
| PA62 | Palmitoyl-IAAAEEEEK(DO3A:Gd)-(PEG)₂-propionic acid | SEQ ID NO: 5 |
| PA63 | Palmitoyl-IAAAEEEEK(DO3A:Gd)-(PEG)₃-propionic acid | SEQ ID NO: 5 |

*Sequence for bolded portion

### Example 3: Probing Peptide Amphiphile Self-Assembly in Blood Serum

One of the great challenges in developing biocompatible, dynamic systems that can transform morphologically in vivo is the difficulty in probing their self-assembly behavior in blood serum. Serum contains variable amounts of salt concentrations, as well as proteins such as albumin and immunoglobulins that bind to amphiphilic molecules, enzymes, and other molecules (Krebs, H. A. Annu. Rev. Biochem. 1950, 19:409; Takeda, K. et al. J. Protein Chem. 1990, 9:17; Turro, N. J. et al. Langmuir 1995, 11:2525; Jones, M. N. Biochem. J. 1975, 151:109). Any spectroscopic technique for determining the self-assembly behavior of PAs in the presence of other proteins will require the addition of a chromophore to the molecular structure. However, even minor changes to the molecular structure of the PA can dramatically change the pH trigger of self-assembly. Indeed, the addition of just a single methyl group in the β-sheet sequence shifted the transition basic by 0.4 pH units (Ghosh, A. et al. J. Am. Chem. Soc. 2012, 134:3647). Thus, developing approaches for probing this transition in serum without significantly altering the self-assembly behavior is an essential prerequisite for understanding the influence of this pH-triggered self-assembly on in vivo biodistribution.

A method was developed to probe the pH-dependent self-assembly morphology of PAs in pure mouse blood serum without significantly changing their intrinsic self-assembly behavior. Conjugating a fluorophore with appropriate lifetimes and excitation and emission spectra to 1.5% of the PA molecules allows for distinguishing between spherical and nanofiber morphologies via fluorescence anisotropy (FA) in pure serum at a detection limit of 10 μM. The molecular crowding of albumin promotes self-assembly of these anionic PAs into nanofibers in serum. Finally, a solution consisting of 150 mM NaCl, 2.2 mM CaCl₂ and 1.8 mM 20 kDa polyethylene glycol (PEG) accurately simulated the ionic strength and crowded environment of pure serum and enabled characterization of self-assembly behavior using circular dichroism (CD) and critical aggregation concentration (CAC) measurements, replicating the transition pH values in pure serum to within 0.08 pH units.

Materials and Methods

Synthesis of Peptides Amphiphiles (PA):

All amino acids used in this example were purchased from AnaSpec Inc. PAs were synthesized on a 0.5 mmol scale with Sieber resin (AAPPTEC) in a linear fashion from the C-terminus to N-terminus direction using Fmoc chemistry. The resin was swollen in a shaker vessel with dichloromethane (DCM) for 30 minutes, the DCM was removed and dimethyl formamide (DMF) was added to the vessel and further shaken for 30 minutes. After the liquid was removed, 10% N,N-Diisopropylethylamine (DIPEA) in DMF was added to the vessel and mixed for 10 minutes. For deprotection, 20% piperidine in DMF was used to remove the Fmoc protecting group on the resin. A Kaiser test protocol confirmed removal of the Fmoc protecting group. Coupling of the acid to the amine end of resin was done through activation using O-Benzotriazole N,N,N′,N′-tetramethyluronium hexafluorophosphate (HBTU) or 2-(7-Aza-1H-benzotriazole-1-yl)-1,1,3,3-tetramethyluronium hexafluorophosphate (HATU). The coupling solution contained 4.0 Eq. of amino acid, 3.96 Eq. of HBTU/HATU, 4 Eq. of N-Hydroxybenzotriazole (HOBt) orl-Hydroxy-7-azabenzotriazole (HOAt), and 8 Eq. of DIPEA with respect to peptide allowing at least 3 hours of coupling per amino acid (also confirmed via the Kaiser test). The surfactant Triton X-100 was added to the coupling solution and to the latter amino acids to aid in coupling efficiency. Resin cleavage of the peptide was done by addition of the following cocktails: a solution of 1% Trifluoroacetic acid (TFA), 1% Triisopropyl silane (TIS), 2% Anisole and 94% DCM was used for the PAs that would be conjugated to DO3A. The TFA was removed under vacuo and the PA was precipitated using two 20 mL portions of either cold water, or a solution of 95% TFA, 1% TIS, 2% Anisole, 2% water was used for the PAs to be conjugated to the Ru(bipy)₃ NHS ester dye. The TFA was removed under vacuo and the PA was precipitated using two 20 mL portions of either cold water for the DO3A PAs or cold diethyl ether for the Ru(bipy)₃-conjugated PAs. The crude peptide was filtered and washed with cold water/cold diethyl ether.

Synthesis and Purification of Tri-Tert Butyl Ester of DO3A:

All reagents were purchased from Sigma Aldrich and used without further purification unless specified. 30 g of cyclen (1,4,7,10-tetraazacyclododecane) was combined with powdered, dry 42.9 g of sodium acetate in 400 mL of N,N-dimethylacetamide in a round bottom flask and stirred for 30 minutes with an overhead glass rod stirrer. The round bottom flask containing the slurry was placed in an ice bath until it reached 0° C. 77.1 mL of tert-butyl bromoacetate was dissolved in 150 mL of N, N,-dimethylacetamide and added drop-wise to the slurry at 0° C. over a period of 25 minutes. The slurry equilibrated back to room temperature and was stirred for 5 days. A separate solution was made by dissolving 30 g of potassium bromide (KBr) in 2 L of deionized water (Millipore) followed by stirring and heating to a temperature of 50° C. After the KBr solution reached 50° C., it was added to the slurry forming a yellow colored solution. The pH was adjusted to 9.0 by the addition of powdered sodium bicarbonate and checked via litmus paper. Precipitation of the desired product, acetic acid tert-butyl ester hydrobromide (4,7-bis-tert-butoxycarbonylmethyl-1,4,7,10-tetraaza-cyclododec-1-yl), settled for 4 hours without stirring, followed by vacuum filtering and drying, yielding a white powder. 10.0 g of acetic acid tert-butyl ester hydrobromide was dissolved in 50 mL of acetonitrile (MeCN) and combined with 5.1077 g (2.2 eq.) of finely powdered, dry potassium carbonate and stirred for 30 minutes. Benzyl bromo acetate 2.927 mL (1.1 eq.) was added drop-wise to the solution and stirred overnight at 50° C.-60° C. The mixture was cooled to room temperature and vacuum filtered, with the desired product in the filtrate. The solid was washed twice with MeCN. The combined MeCN was removed by evaporation under vacuum, yielding the tri-tert-butyl ester form of DO3A, a viscous yellow gel. NMR and ESI-MS confirmed the presence of the desired product. The crude product was purified using flash-column chromatography using 50.0 g of silica gel for every 1.0 g of tri-tert-butyl ester form of DO3A using DCM as the mobile phase. The product was eluted from the column using a gradient elution, starting with 2% MeOH in DCM to 6% of MeOH in DCM. The elution of the desired product was followed by TLC, using 10% MeOH in DCM as the mobile phase. Pure fractions were combined and the solvents evaporated under vacuum. The residue was then dissolved in approximately 50 mL of MeOH in deionized water (Millipore) at a ratio of 9:1. Palladium on carbon catalyst was added to the solution in 20% by weight with respect to tri-tert-butyl ester form of DO3A. The sample was hydrogenated under 50-psi hydrogen pressure overnight followed by filtration of the solid catalyst. The filtrate containing DO3A was evaporated under vacuum to remove the methanol then 100 mL of deionized water was added to the solution. Diethyl ether (50 mL) was added 3 times to the solution in a separatory funnel to extract the non-hydrogenated product. Solvent was removed by evaporation and the solution was freeze-dried to remove remaining deionized water, yielding a yellowish powder. NMR and ESI-MS were used to confirm the presence of DO3A and check purity.

Conjugation of PAs with Tri-Tert-Butyl Ester Form of DO3A:

The PAs (Palmitoyl-IAAAE(tert-butyl)₄K(ε-NH₂)—NH₂ (SEQ ID NO:38, bolded portion) or Palmitoyl-MAAAE(tert-butyl)₄K(ε-NH₂)—NH₂) (SEQ ID NO:39, bolded portion) were dissolved in pyridine followed by addition of 2 eqv. of tri-tert-butyl ester form of DO3A, 2 eqv. of HATU and 4.4 eqv. of DIPEA. The solution was left to stir overnight. ESI-mass spectrometry was used to monitor conjugation. The product was then precipitated with cold water, filtered and dried under vacuum. The dried solid was then dissolved and stirred in 20 ml of 95% TFA, 1% TIS, 2% water and 2% anisole for about 20-24 hours to remove the tert-butyl groups on the glutamic acids in PA and the carboxylic acids in DO3A. The excess TFA was removed under vacuum. The final de-protected product (PA-DO3A) was precipitated with cold ether, filtered and dried under vacuum before HPLC purification.

Purification of PAs:

The crude PAs were dissolved in 0.1% NH₄OH v/v (aq) at approximately 10-20 mg/mL by vigorously shaking and sonicating until the solution turned clear. To aid in dissolution, an additional drop of concentrated NH₄OH was added to the solution. The PA solution was filtered first using a 0.45 μm syringe filter (Whatman), followed by a 0.2 μm syringe filter. The sample was purified on a Shimadzu preparative HPLC (dual pump system controlled by LC-MS solution software) with an Agilent PLRP-S polymer column (Model No. PL1212-3100 150 mm×25 mm) under basic conditions. The product was eluted with a linear gradient of 10% to 100% MeCN containing 0.1% NH₄OH (v/v) over 60 minutes. The purity of the collected fraction was verified using an electro-spray ionization (ESI) time-of-flight mass spectrometer (Bruker) and a Shimadzu Analytical HPLC system. Fractions greater than 95% purity were combined; the MeCN was removed by vacuum before freeze-drying.

Chelation of PA-DO3A with Gd³⁺:

10-15 mg of previously prepared and purified PA-DO3A was dissolved in 1.0-2.0 mL of water and combined with ˜3 mL of 2 eq. of GdCIl in 0.01 M HCl. The reaction was set to stir in an oil bath at 60° C. for 30 min. The pH of the solution was gradually adjusted from approximately pH 3.0 to 5.0-5.5 (Litmus paper) using small amounts of 0.050 M NaOH. The resultant solution was stirred for 24 hours at 60° C. A small sample was removed and analyzed by ESI-mass spectrometry to determine the extent of reaction completion. The pH of the solution was then raised over a period of an hour using ammonium hydroxide to precipitate excess Gd³⁺ as Gd(OH)₃, then filtered using a 0.2 μm syringe filter. The solution was dialyzed against de-ionized water to remove NaCl and any remaining free Gd³⁺ using a SpectraPor Biotech cellulose ester dialysis membrane with 100-500 D MW cut-off. The de-ionized water for dialysis was changed 4-6 times over a period of 24 hours. The PA-DO3A:Gd solution was freeze-dried to recover a white fluffy powder with a final yield of ˜25%.

Conjugation of PAs and Mouse Serum Albumin with Ru(Bipy)₃:

Bis(2,2′-bipyridine)-4′-methyl-4-carboxybipyridine-ruthenium N-succinimidyl ester-bis(hexafluorophosphate) was purchased from Sigma Aldrich and used without further purification. 5 mg of the dye was dissolved in 500 μL of dimethyl sulfoxide (DMSO) to make a stock solution. 10-15 mg of PAs were dissolved in 1 mL of 0.2 M NaHCO₃ (aq.) solution and stirred for 2-3 hours, followed by slow addition of 100 μL of the dye stock solution. The reaction solution was left to stir overnight at room temperature. ESI-mass spectrometry and analytical HPLC were used to track the coupling. Excess free dye was removed by dialysis (500-1000 Da cut-off Millipore tubing) against de-ionized water. The PA-(Rubipy)₃ was then separated from unreacted PAs via analytical HPLC using a linear gradient of 10% to 100% MeCN containing 0.1% NH₄OH (v/v) over 30 minutes. The same procedure was followed for conjugation of the dye to mouse serum albumin (MSA-Sigma Aldrich) except no analytical HPLC purification was done.

Circular Dichroism (CD) Spectroscopy:

Measurements were done on a Jasco-815 Circular Dichroism spectrometer using 0.5-1 cm path length quartz cuvettes. 500-10 μM of either pure PA or PA mixtures were prepared in 150 mM NaCl and 2.2 mM CaCl₂ (and 1.8 mM 20 kDa PEG in select samples) by dilution from a concentrated PA stock (0.5-1 mM, pH 9). Double de-ionized Milli-Q water was used for preparing all solutions. The solutions were then heated at 80° C. for 30 minutes in a water bath and gradually cooled to room temperature. An Accumet XL15 pH meter (Fisher Scientific) coupled with an Orion Ross Ultra semi-micro electrode (8103BNUWP, Thermo Scientific) was used to adjust the pH of the solution to the desired value followed by collection of the CD spectra. Each trace shown was averaged over 3 accumulations and was baseline subtracted using aqueous solutions containing salts only. All spectra are cut-off below 200 nm due to absorption by NaCl and CaCl₂. The same procedure was followed for the salt control samples with 3.0 and 4.0 mM CaCl₂.

Transmission Electron Microscopy (TEM):

TEM images were obtained using solutions of the 100 μM PA mixture concentration in 1.5% (v/v) serum solution and a serum control. The PAs, however, were not heated in this case to avoid destroying/denaturing serum proteins. This was followed by pH adjustment using either HCl or NaOH. 5 μL of this solution was pipetted onto a Carbon Formvar grid (Electron Microscopy Sciences) and allowed to sit for 2 minutes before being wicked dry using filter paper. The samples were then negatively stained using 1 wt % uranyl acetate and imaged under a FEI Tecnai G2 Bio TWIN TEM system, operating at 100 kV. All TEM experiments were performed in duplicate.

Critical Aggregation Concentration (CAC) Determination Using the Pyrene 1:3 Method:

For CAC measurements, a series of solutions of either pure PA or PA mixture with concentrations ranging from 100-300 nM to 500-700 μM were prepared using serial dilutions in 150 mM NaCl and 2.2 mM CaCl₂ (and 1.8 mM 20 kDa PEG in select samples). The final concentration of pyrene in each solution was fixed to be 4.5 μM. This was followed by pH adjustment of the solutions using careful additions of HCl or NaOH. 100 L of each solution was transferred to a 96-well plate and the fluorescence emission of pyrene was monitored using a hybrid reader fluorimeter (BioTek Synergy H4) at room temperature. The excitation wavelength was set at 335 nm. The ratio of the intensities of emissions at 376 nm and 392 nm were then plotted as a function of the PA concentration (log scale). The CAC was determined from an abrupt change in the slope of the plot using the least-squares fitting technique.

Fluorescence and Fluorescence Anisotropy (FA):

Fluorescence and FA measurements were done using 20-100 μM of PA mixtures in 150 mM NaCl and 2.2 mM CaCl₂ and various serum (MP Biomedicals) concentrations (0.75-100% v/v) diluted in the same salt buffer. PA samples in serum or MSA were not heated and cooled. All samples were then pH adjusted using HCl/NaOH solutions, transferred to a 96-well plate followed by collection of fluorescence emission from PA-(Rubipy)₃ first parallel (Ipar) and then perpendicular (Iperp) to the excitation polarization using a hybrid reader fluorimeter (BioTek Synergy H4) at room temperature. The excitation wavelength used was 458 nm and the emission was monitored using a 640/20 nm filter. The same method was followed for control experiments involving serum background samples, dye conjugated MSA, only 1.5% PA-(Rubipy)₃ in serum and 100 μM PA mixture in MSA and salt buffer. FA was calculated using the following equation:

$$FA = \frac{( Ipar - Iperp )}{( Ipar + ( 2 * Iperp ) )}$$

and plotted as a function of either pH or time.

Results

Model PA Systems

Palmitoyl-IAAAEEEEK(DO3A:Gd)—NH₂ (PA5) (SEQ ID NO:5, bolded portion) and Palmitoyl-MAAAEEEEK(DO3A:Gd)—NH₂ (PA65) (SEQ ID NO:40, bolded portion) (Table 13) were synthesized as model PA systems (DO3A=1,4,7-tris(carboxymethylaza)cyclododecane-10-azaacetyl amide). PA5 was previously found to exhibit a concentration independent transition from spherical micelles to nanofibers in 150 mM NaCl and 2.2 mM CaCl₂ at a pH of 6.0 (Ghosh, A. et al. J. Am. Chem. Soc. 2012, 134:3647). To distinguish between the spherical and nanofiber morphologies via FA, bis(2,2′-bipyridine)-4′-methyl-4-carboxybipyridine ruthenium(II) (Rubipy) was conjugated to the lysine ε-amine group of Palmitoyl-IAAAEEEEK-NH₂ (PA66) (SEQ ID NO:5, bolded portion) and Palmitoyl-MAAAEEEEK-NH₂ (PA67) (SEQ ID NO:40, bolded portion) via NHS ester linkers (Table 13). Chart I illustrates the chemical structures of PA65 and PA67. For FA measurements. PA66 and PA67 were spiked into PA5 and PA65, respectively (PAmix1 and PAmix2) in a small amount (1.5% of the total PA concentration).

Ru(bipy)₃²⁺ was chosen as the FA fluorophore for two reasons. First, it has an excitation wavelength of 458 nm which is significantly red-shifted from that of serum proteins, thus minimizing serum auto-fluorescence. Second, the fluorescence lifetime of Ru(bipy)₃²⁺ (˜400-500 ns) is sufficient to distinguish between the spherical micelle and nanofiber morphologies via FA. FA measures the extent of decorrelation of the polarized emission from a fluorescent dye with respect to the polarization of the excitation light, which linearly depends on rotational correlation time of the dye-containing rotating unit in solution and consequently its molecular weight (Ameloot, M. et al. Pure Appl. Chem. 2013, 85:589; Owicki, J. C. J. Biomol. Screening 2000, 5:297). The FA value reflects an average molecular weight distribution of the entire ensemble of PA nanostructures. The unassembled, isolated PAs have molecular weights of ˜1.8 kDa. A 10 nm spherical micelle formed from these PAs with an estimated aggregation number of 60-100 (˜108-180 kDa) (Turro, N. J. et al. J. Am. Chem. Soc. 1978, 100:5951; Tsonchev, S. et al. J. Phys. Chem. B 2008, 112:441) would have a different FA than a micron-sized nanofiber, which has a molecular weight orders of magnitude larger and scaled according to length. For example, a 500 nm long fiber is expected to have a molecular weight of 5-9 MDa. The rotational correlation times of a spherical micelle and a nanofiber would be 50-100 ns and >1 ms, respectively (Yguerabi, J. et al. J. Mol. Biol. 1970, 51:573).

TABLE 13
Synthesized PA molecules

| Molecule | Sequence | SEQ ID NO:* |
|---|---|---|
| PA5 | Palmitoyl-IAAAEEEEK(DO3A:Gd)-NH₂ | SEQ ID NO: 5 |
| PA65 | Palmitoyl-MAAAEEEEK(DO3A:Gd)-NH₂ | SEQ ID NO: 40 |
| PA66 | Palmitoyl-IAAAEEEEK-Rubipy | SEQ ID NO: 5 |
| PA67 | Palmitoyl-MAAAEEEEK-Rubipy | SEQ ID NO: 40 |

*Sequence for bolded portion
PA structure and design:

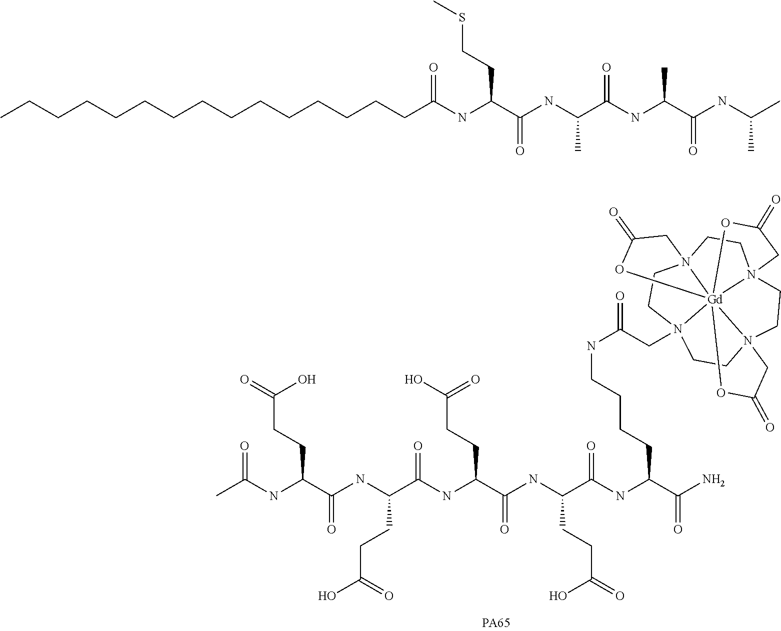

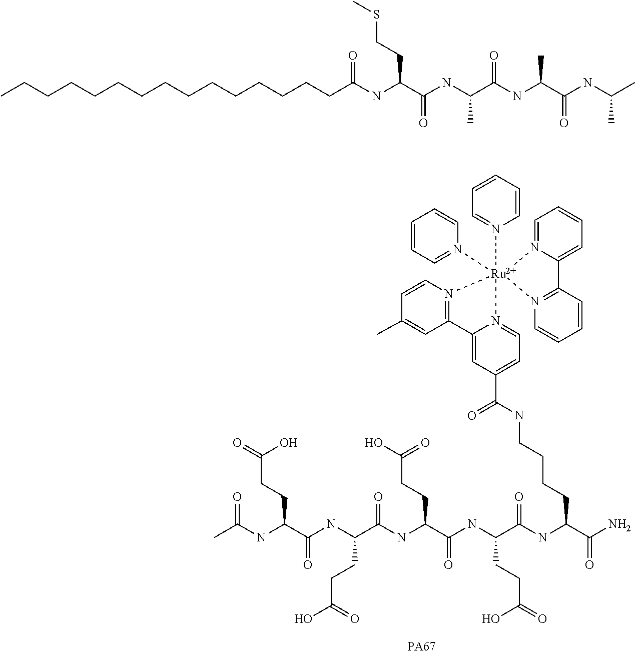

Phase Diagram of PA Mixture and Reversibility of Morphology Transition.

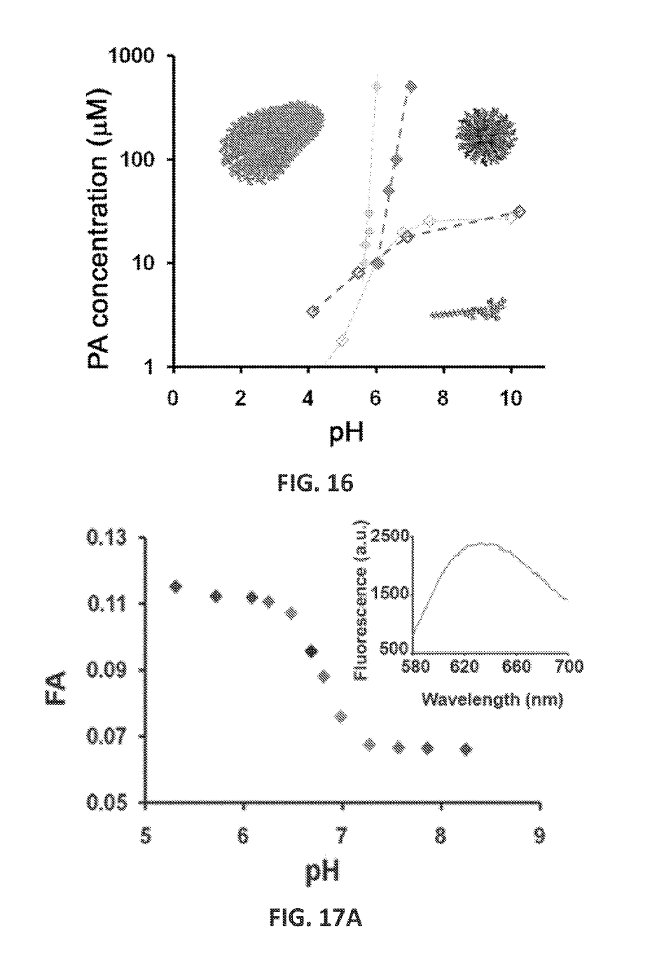

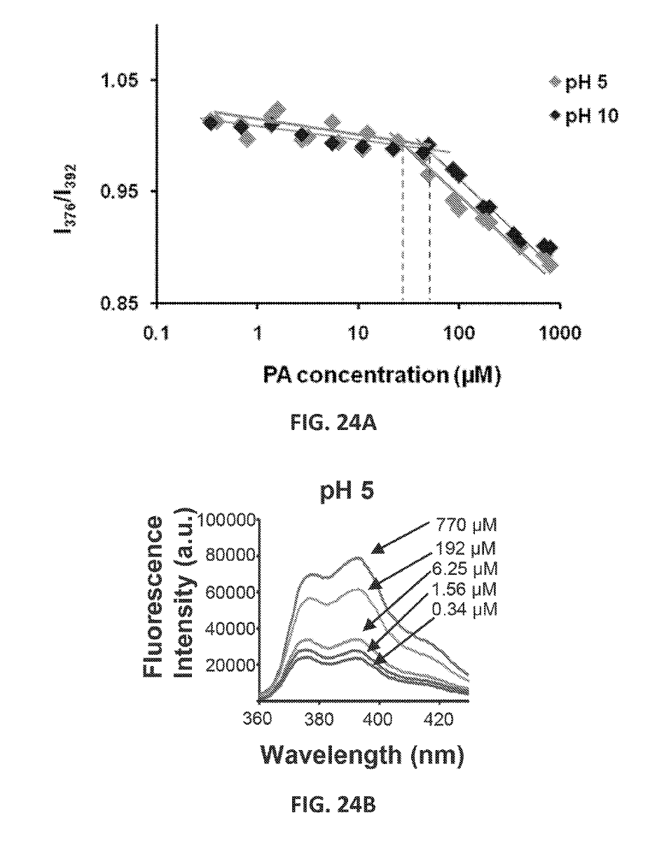

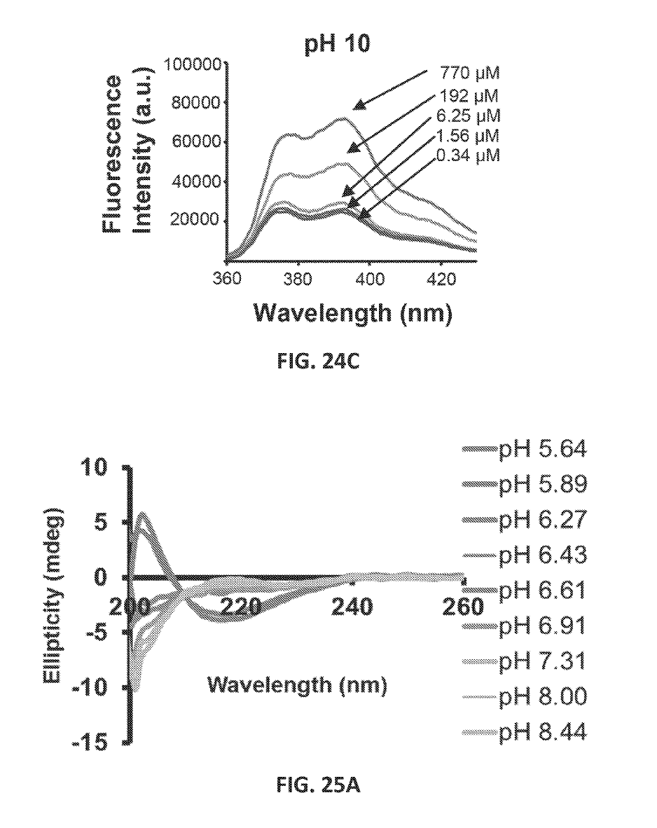

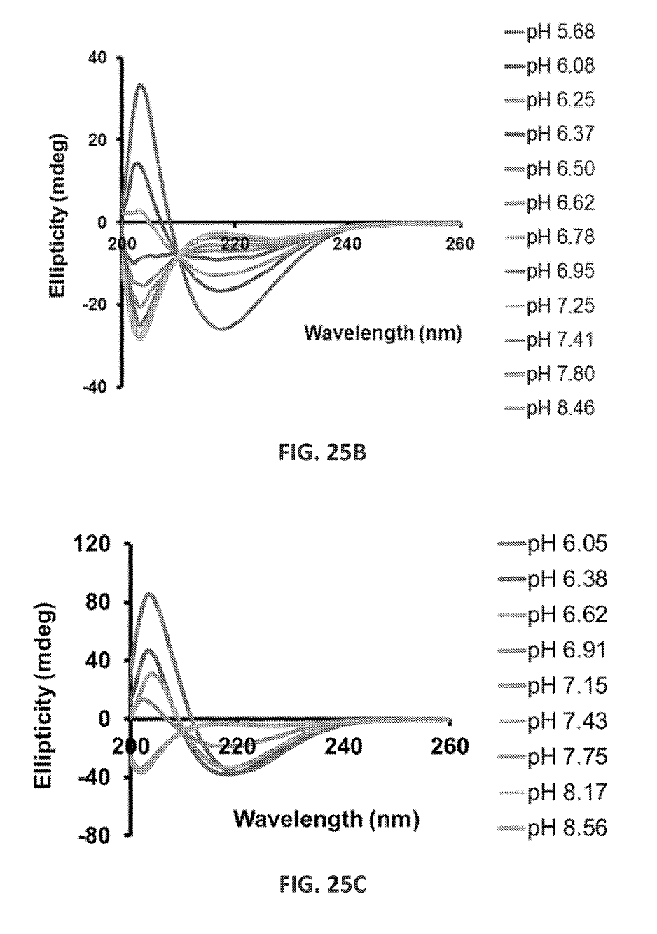

Self-assembly behavior is known to be extremely sensitive to minor changes in monomer structure and presence of impurities. To ensure that the addition of 1.5% PA66 to PA5 does not significantly disrupt self-assembly behavior, a concentration-pH phase diagram was mapped for PAmix1 in a 150 mM NaCl and 2.2 mM CaCl₂ salt solution and compared with the phase diagram for pure PA5 (FIG. 16). CAC measurements via the pyrene 1:3 method (Das, D. et al. J. Colloid Interface Sci. 2008, 327:198) were used to ascertain the single molecule to spherical micelle transitions at varying pH values (FIGS. 24A-24C). CD Spectroscopy was used to determine the pH points at which different concentrations (10-500 μM) of the PA molecules transitioned from a random coil to a β-sheet secondary structure (FIGS. 25A-25C). It has been previously shown that the random coil and β-sheet structures correspond to spherical micellar/isolated molecule and nanofiber morphologies respectively (Ghosh, A. et al. J. Am. Chem. Soc. 2012, 134:3647). The CD transition point was defined to be the pH value at which the ellipticity at 200 nm rose from a negative value (random coil) to zero accompanied by the appearance of a minimum at 218-220 nm. The addition of PA66 shifted the CD transition pH to more basic values by ˜0.6 pH units. The CAC values, on the other hand were found to be 5× higher in the acidic region but comparable under basic conditions relative to pure PA5. This ˜0.6 pH shift in CD transition pH to more basic values were also observed when 1.5% of PA67 was added to PA65.

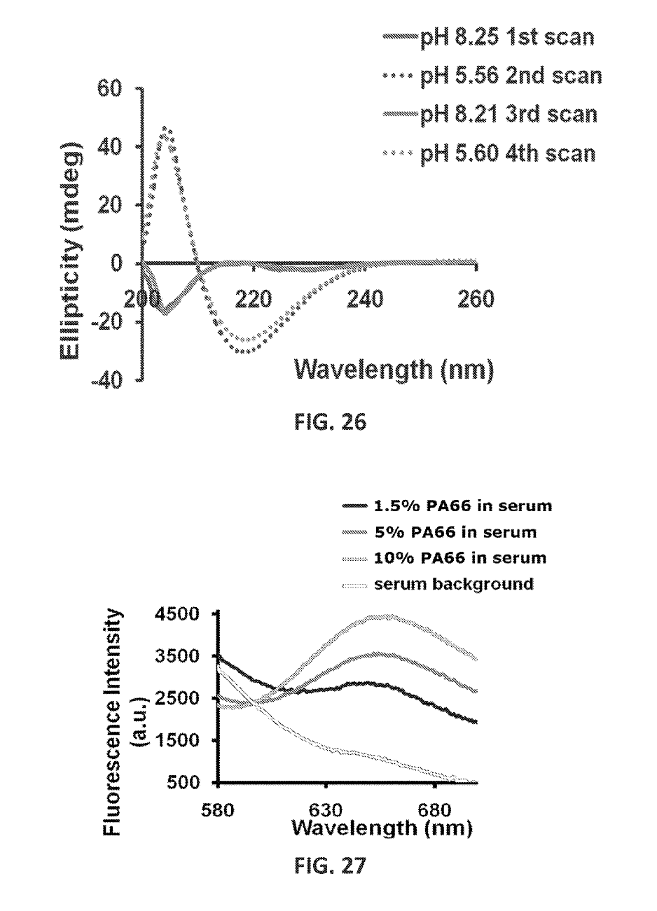

The transition was found to be rapid and reversible with respect to pH. This was tested by switching the pH of the PA mixture back and forth between ˜5.0 and -10.0 followed by collection of the CD spectra within 2-3 min (FIG. 26). The CD curves showed almost superimposable secondary structures for the acidic and basic pH values, indicating that these structures exist in thermodynamic equilibrium.

FA-CD Correlation.

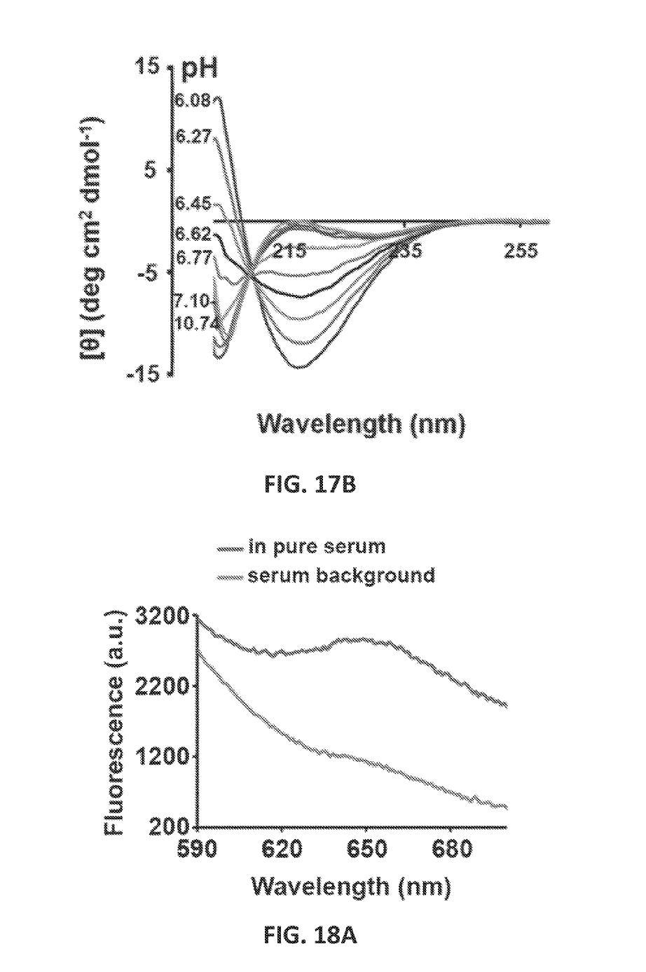

pH dependent FA values of a 100 μM PAmix1 in the same isotonic salt solution (FIG. 17A) were then obtained. The inset in FIG. 17A shows the fluorescence emission from PA66 in the mixture upon excitation with 458 nm light that was exploited for FA measurements. The FA values increased from 0.066 to 0.115 as the pH was carefully lowered from 8.35 to 5.31. The onset of the self-assembly transition was defined by the data point that first showed a higher value relative to the constant FA at basic pH values. The end of this transition was defined as the pH point below which the FA became constant again. The transition profile measured via FA correlated well with that obtained from the pH dependent CD spectra (FIG. 17B). The FA transition onset at 6.98 is close to the most basic pH CD spectrum at 7.10 that started to deviate from a superimposable random coil morphology. Also, the FA transition midpoint at a pH of 6.68 was close to our previously defined CD transition spectrum at a pH of 6.62.

FA in Pure Serum.

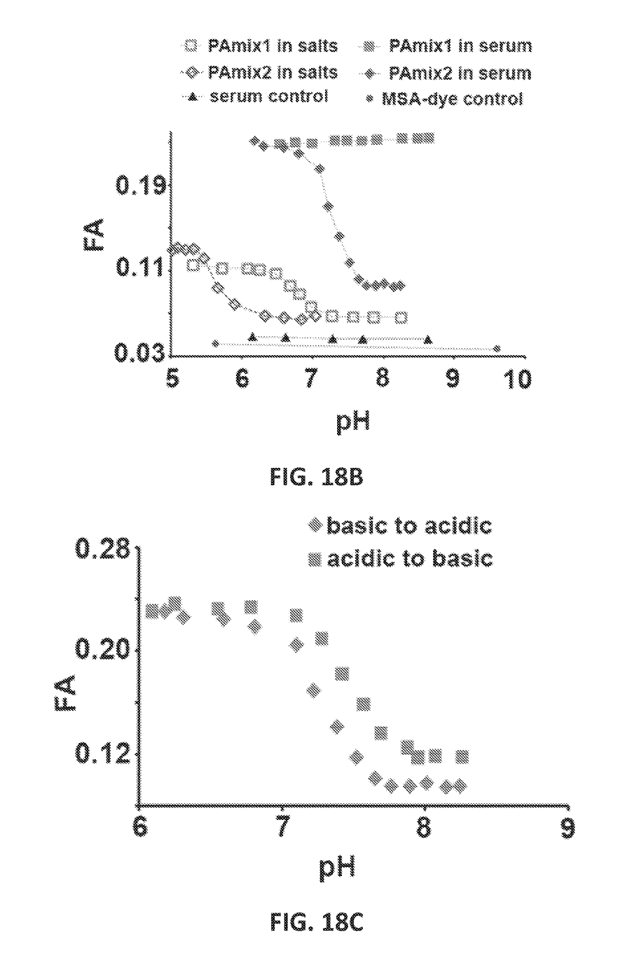

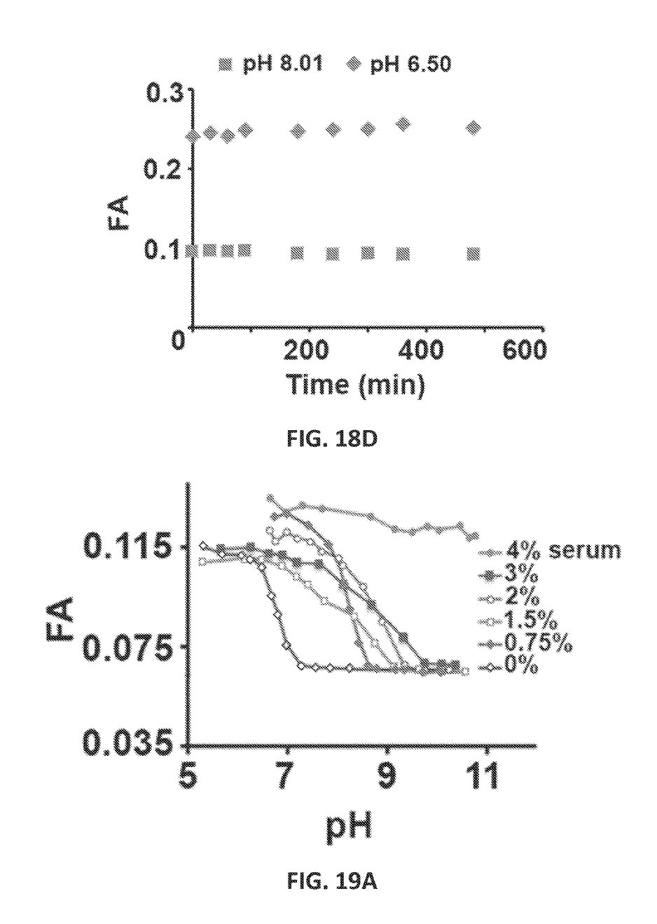

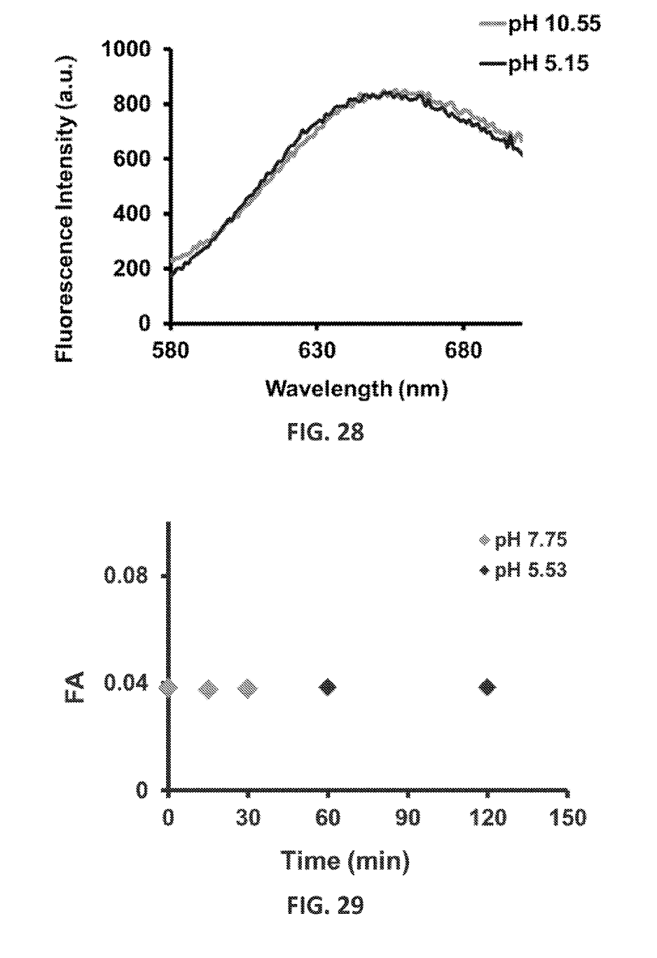

The fluorescence of both PA mixtures was detectable in pure mouse serum above the serum autofluorescence background. FIG. 18A shows this fluorescence emission from PAmix1 in blood serum along with the serum auto-fluorescence background. The fluorescence emission intensity increased with increasing amounts of PA66 in serum (FIG. 27) and was found to be superimposable under acidic and basic conditions (FIG. 28) in the simulated salt solution. The ˜30 nm red shift in the PA66 fluorescence emission in serum is commonly observed for Ru(bipy)₃²⁺ in the presence of protein and lipid molecules due to changes in the dye's emission pathways (Innocenzi, P. et al. J. Phys. Chem. B 1997, 101:2285). For a 100 μM PAmix1, the FA in pure mouse blood serum was found to be pH independent with a constant value of ˜0.22 as shown in FIG. 18A. This constant high value results from the formation of nanofibers immediately after addition of the serum to the PA mixture even at basic pH values. On the contrary, PAmix2 was found to transition in the pH range 7.1-7.4 as evident from the increase in FA values from 0.09 to 0.23, also shown in FIG. 18B. Comparing the serum results with the FA of PAmix2 in the salt solution (FIG. 18B) indicates that serum shifts the transitional pH to more basic values. This shift is significantly more prominent for PAmix1 which goes from transitioning at pH 6.6 in salts (FIG. 18B) to a constant nanofiber morphology, most likely due to the stronger β-sheet propensity of Isoleucine.

To confirm that this pH dependent jump in FA values in the serum samples was actually reflecting changes in self-assembly morphology and not just the PA66 or PA67, single molecules bound to the 70 kDa serum albumin proteins, a control FA experiment was conducted with a sample containing only 1.5% PA67 isolated molecules in serum (FIG. 18B). The FA values remained constant at ˜0.05 independent of pH. A second control where the Ru(bipy)₃²⁺ dye was directly conjugated to pure Mouse Serum Albumin (MSA) also showed a constant FA value of ˜0.04 over pH and time (FIG. 18B, FIG. 29). Also, FA values obtained for the serum only background ranged from 0.003-0.005, indicating that there is negligible contribution from serum auto-fluorescence.

The general increase in the absolute FA values in serum (˜0.09-0.23) relative to salts (˜0.06-0.11) is attributed to an increase in solution viscosity which increases rotational correlation times and consequently FA. The transition in PAmix2 was rapid (˜3 min after pH change) and reversible with a small hysteresis of 0.3 pH units as shown in FIG. 18C, further confirming that these morphologies are close to thermodynamic equilibrium in pure serum. Stability of the nanostructures in serum were tested for 100 μM PAmix2 via FA over time and shown in FIG. 18D. At pH values of 8.01 and 6.50, the FA was constant at ˜0.09 and -0.23 respectively over a period of 500 minutes. No further change in FA values was observed after 8 days, which highlights the stability of these self-assembled morphologies in serum.

FA in Diluted Serum.

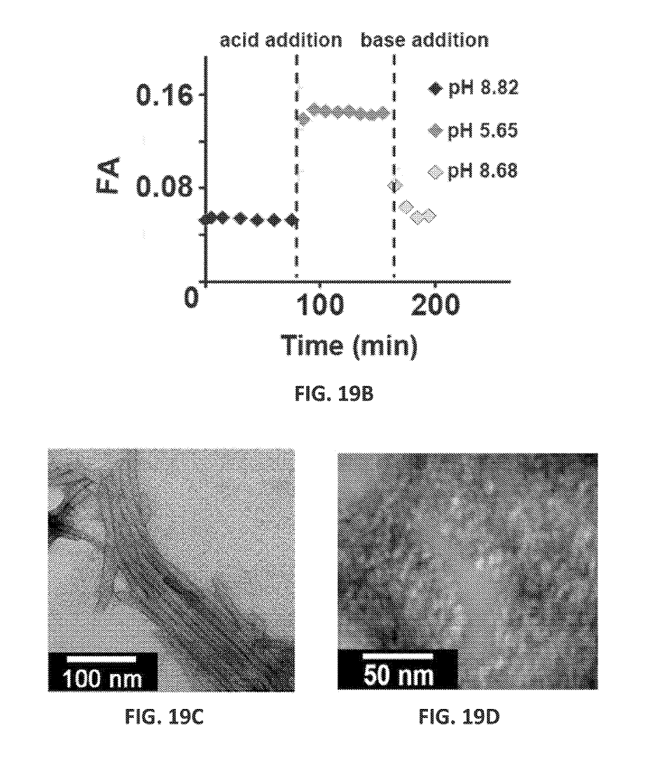

To elucidate the observed drastic influence of serum on the self-assembly behavior of PAmix1, pH dependent FA values were collected for the 100 μM PA mixtures in solutions containing diluted serum concentrations (0.75-4% serum v/v in 150 mM NaCl, 2.2 mM CaCl₂) (FIG. 19A). The transition onset and endpoints shifted to more basic values upon addition of greater concentrations of serum, until no transition was observed in 4% serum. Similar to the values observed in the salt solution, the FA increased from ˜0.065 to ˜0.12 as the pH was lowered. At 4% serum, the FA was found to be constant at ˜0.125, indicating nanofiber formation at and beyond this serum concentration. The pH-dependent transition at lower serum concentration again occurred reversibly within 3 minutes of pH adjustment as shown in FIG. 19B.

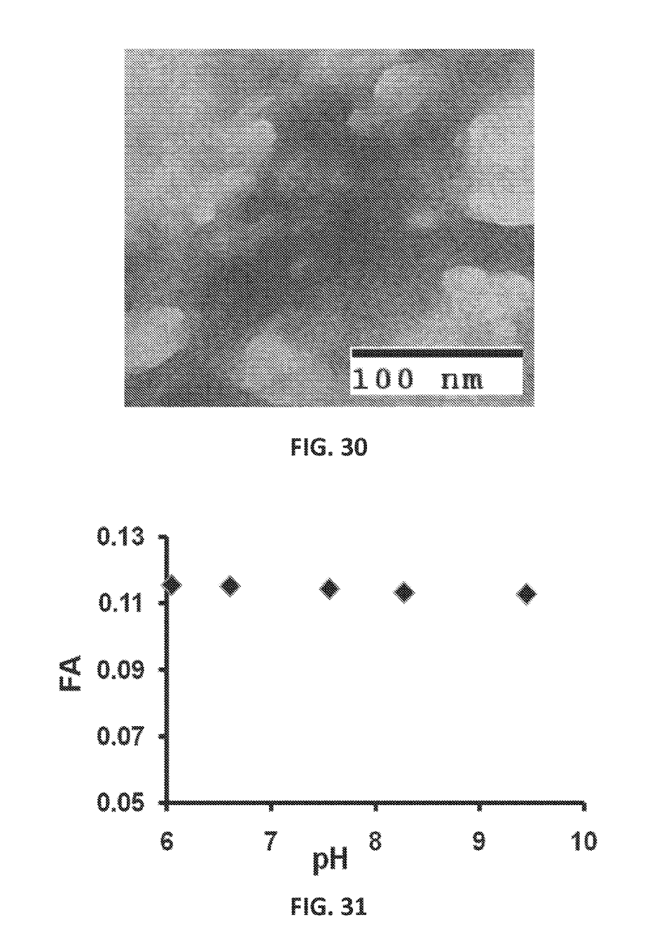

The self-assembly morphologies were further confirmed via conventional Transmission Electron Microscopy (TEM) (FIGS. 19C, 19D) of the 100 μM PAmix1 at pH 6.85 (FA-0.11) and 9.21 (FA-0.06). At pH 6.85, nanofibers having diameters of ˜12.3±1.9 nm were observed. At a pH value of 9.21, a distinctly different globular morphology having diameters of 10.7±1.4 nm was observed, resembling spherical micelles that are typically only distinguishable in salt solutions at much larger PA concentrations (0.5-1 mM) (Ghosh, A. et al. J. Am. Chem. Soc. 2012, 134:3647). A control sample containing 1.5% serum only at a pH 5 showed no fibers (FIG. 30). This imaging data proves that the higher FA values (˜0.11) correspond to the nanofiber morphology.

Effect of Serum Albumin on Self Assembly.

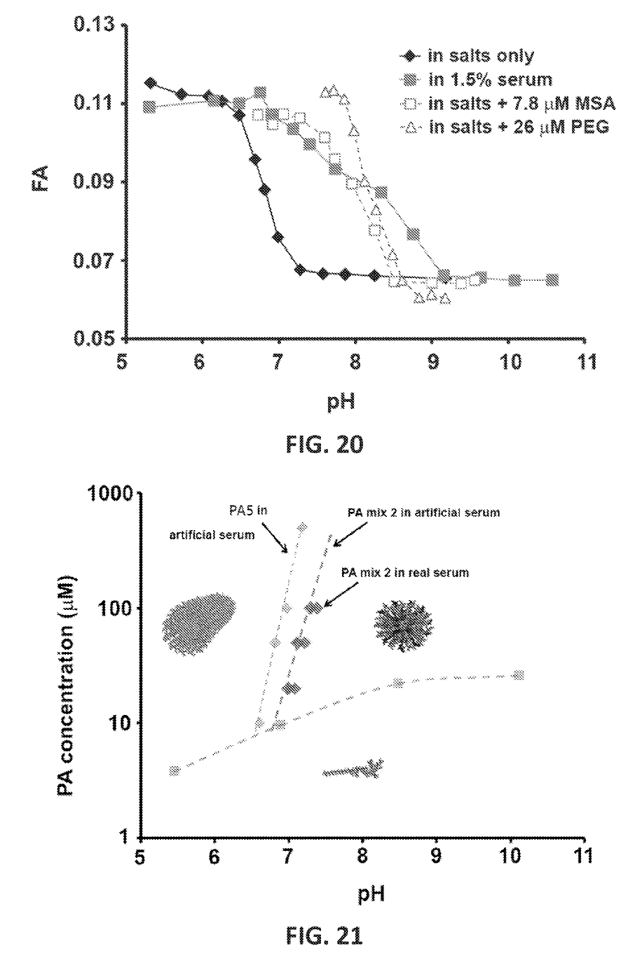

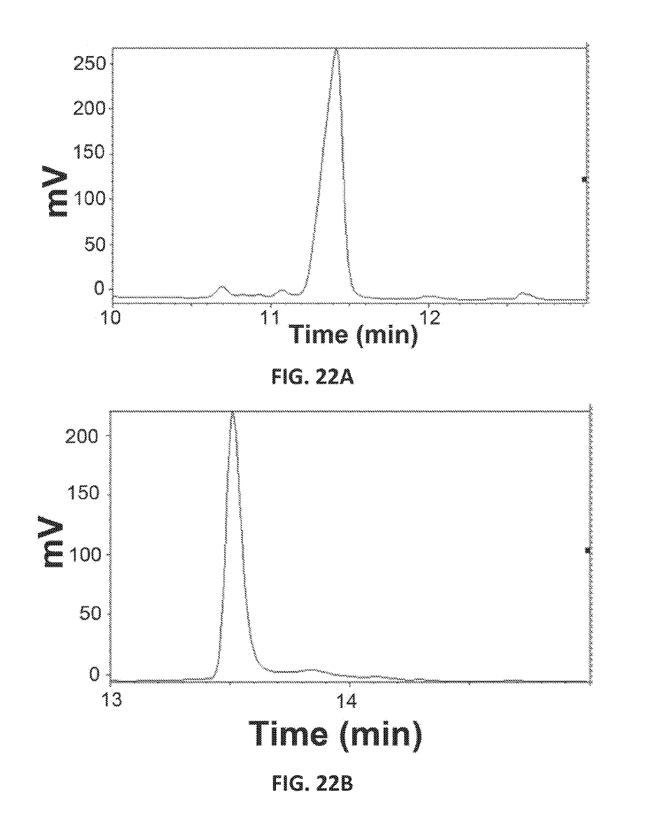

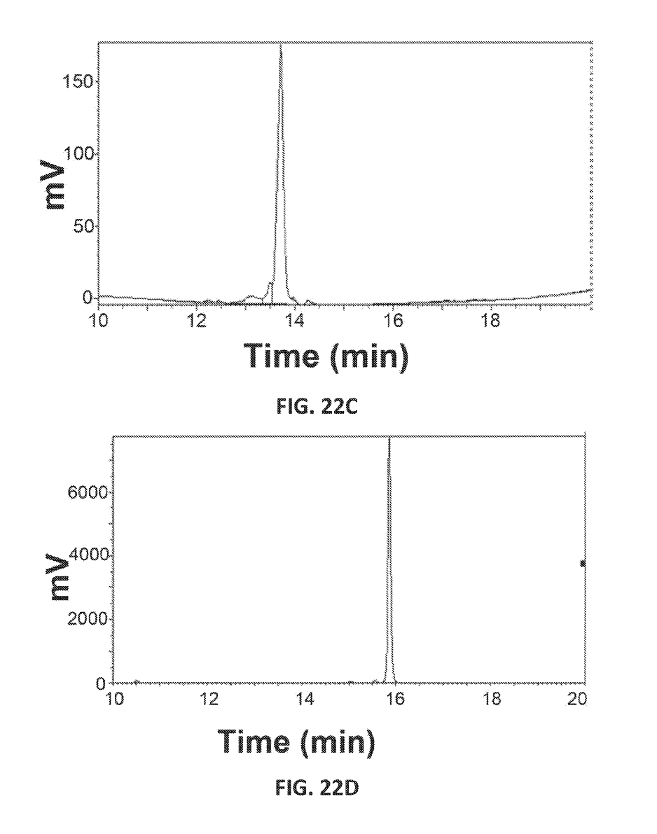

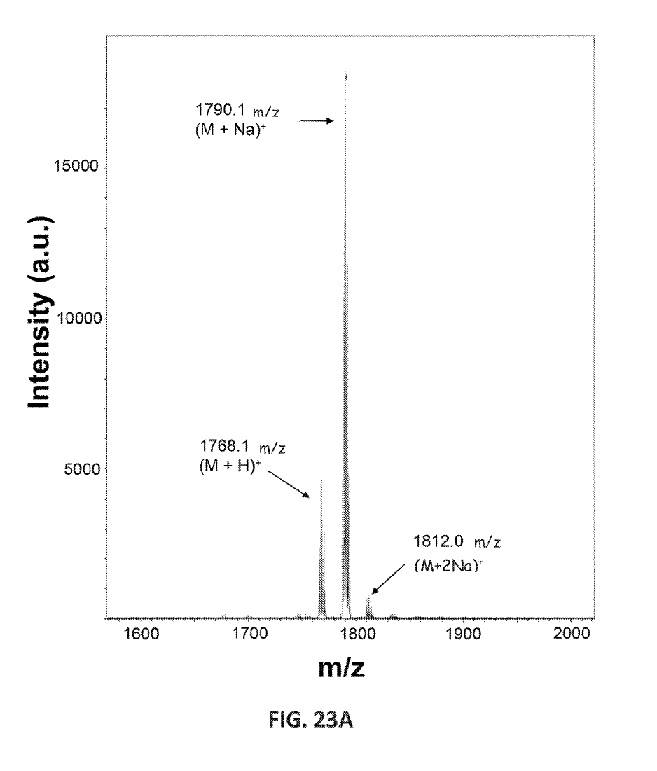

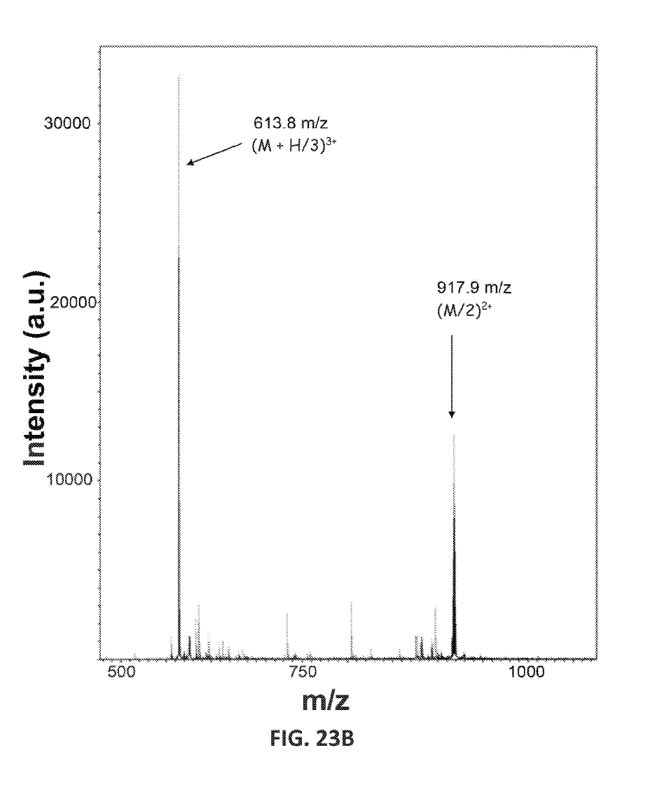

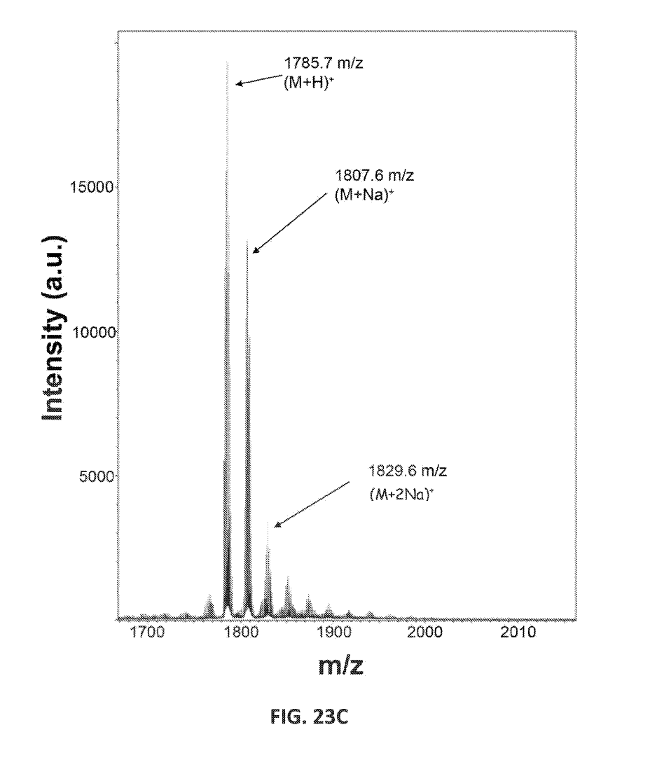

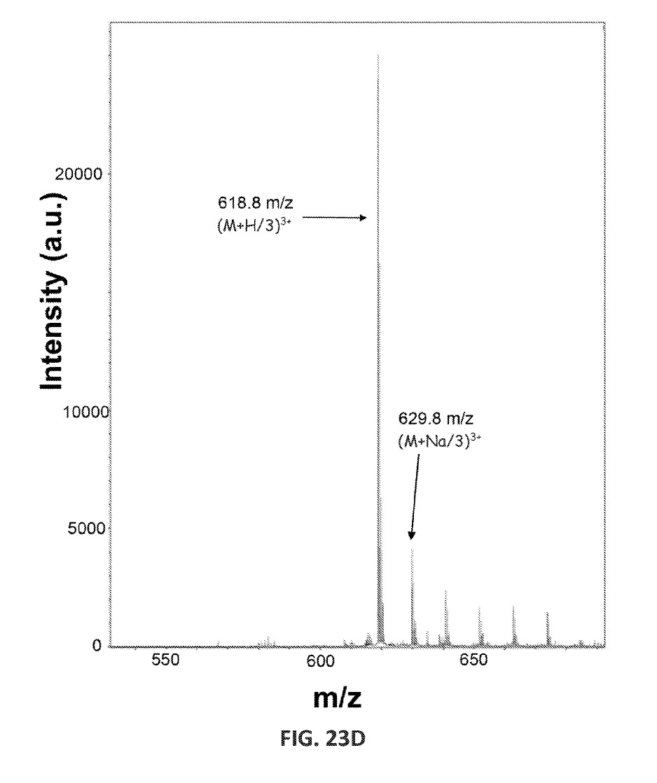

The increased propensity for nanofiber formation in serum could be due to at least two probable factors: interaction with serum proteins and/or a higher ionic strength relative to our salt solution. Serum albumin typically constitutes ˜75-80% of all proteins in blood serum, having a concentration of 35-50 g/L (Krebs, H. A. Annu. Rev. Biochem. 1950, 19:409). It is well known that serum albumin disrupts micelle formation via adsorption of isolated surfactant amphiphiles (Chen, H. et al. Langmuir 2008, 24:5213; Lu, J. et al. Macromolecules 2011, 44:6002). If this occurred in the sample, a decrease in the concentration of PAs in solution, and consequently a shift in transition to lower pH values, would be expected (Ghosh, A. et al. J. Am. Chem. Soc. 2012, 134:3647). To determine the extent to which serum albumin impacts the transition shift, a 100 μM PAmix1 sample was prepared in 7.8 μM MSA, 150 mM NaCl and 2.2 mM CaCl₂. This MSA concentration corresponds to the amount that would be present in 1.5% serum. pH dependent FA (FIG. 20) measurements of the sample showed a transition onset to pH values that were 1.1-1.3 pH units more basic than without MSA, resulting in a transition that occurs within 0.5 pH units of the 1.5% serum sample. This shows that the presence of serum albumin strongly facilitates fiber formation. This observation can be attributed to a crowding effect (Minton, A. P. Curr. Opin. Struct. Biol. 2000, 10:34; Ellis, R. J. Curr. Opin. Struct. Biol. 2001, 11:500). Here, a high concentration of large molecules like albumin promotes self-assembly into a more compact morphology (Minton, A. P. Curr. Opin. Struct. Biol. 2000, 10:34). This was further confirmed via pH dependent FA using the 20 kDa PEG in place of MSA (FIG. 20). A PEG concentration of 26 μM, normalized to the MSA concentration of 7.8 μM by the ratio of their molecular weights, caused a comparable shift in the transitional pH of 100 μM PAmix1. Also, in 1.8 mM PEG which corresponds to the molecular weight normalized MSA concentration in pure serum, nanofibers formed exclusively irrespective of pH (FIG. 31) consistent with pure serum.

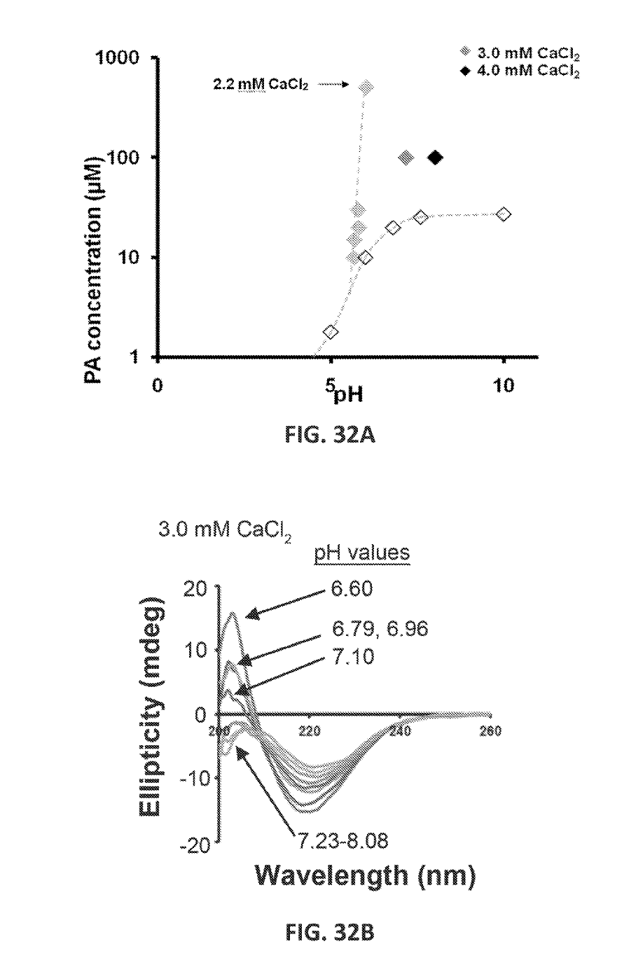

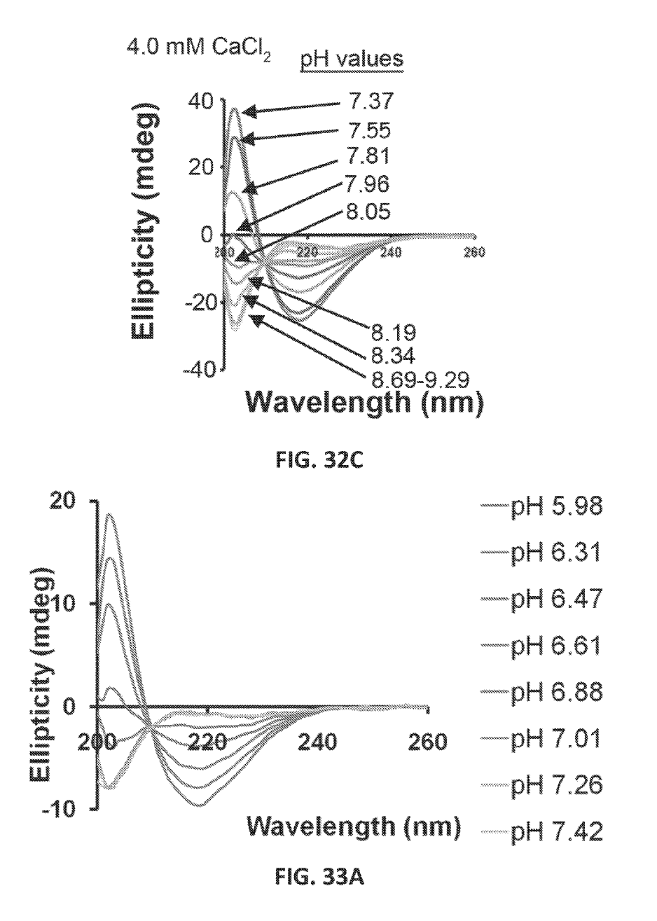

This increased propensity for nanofiber formation in serum is not due to the variability of ionic strength of serum relative to the salt solution. To determine the influence of salt concentration on the transition, the CD transition points of 100 μM PAmix1 in 150 mM NaCl solutions containing 3.0 and 4.0 mM CaCl₂ (FIGS. 32A-32C) were measured. The CaCl₂ concentration was varied since divalent cations more drastically affect the critical coagulation concentrations of amphiphiles (Israelachvili, J. N. Intermolecular and Surface Forces; 2nd ed.; Academic Press: San Diego, Calif., 1992). With 3.0 and 4.0 mM CaCl₂, the transition onset was shifted to more basic pH values by 1 to 2 pH units, respectively, close to the shifts observed in the 0.75 and 1.5% serum samples. Had our observed shifts in the serum samples been due to the greater Ca²⁺ concentration, this would imply that pure serum would have to contain ˜267 mM Ca²⁺. This incredibly high value is completely outside the known range of divalent salts in serum (Krebs, H. A. Annu. Rev. Biochem. 1950, 19:409). This further suggests that the observed transition shifts are primarily due to interactions with serum albumin, with a minor effect from variability in ionic strength.

Phase Diagram in Artificial Serum.

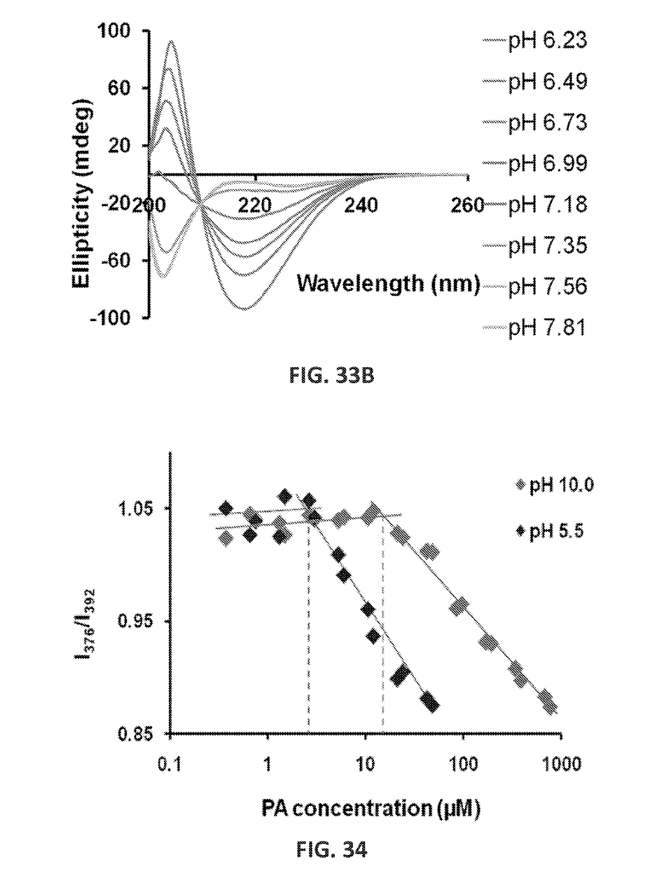

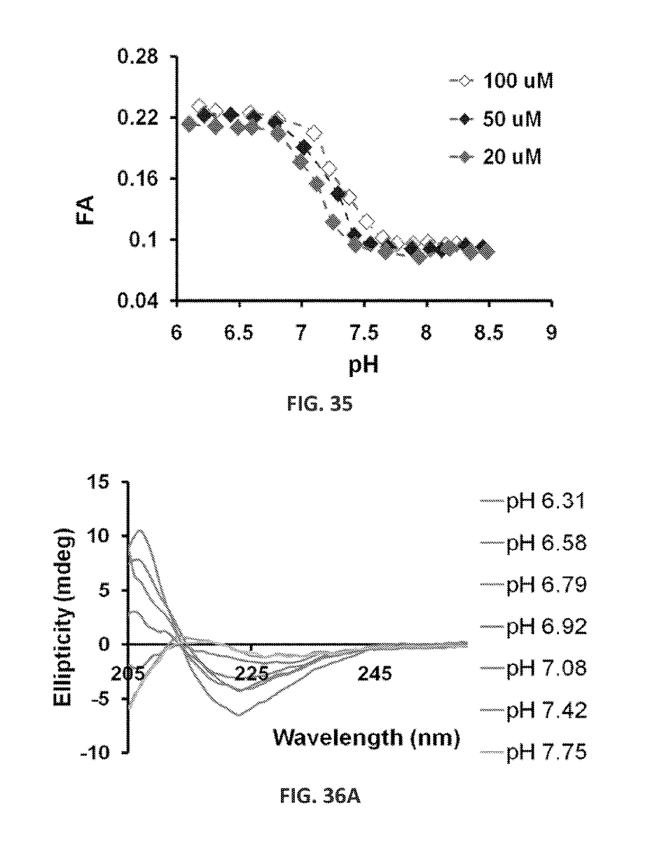

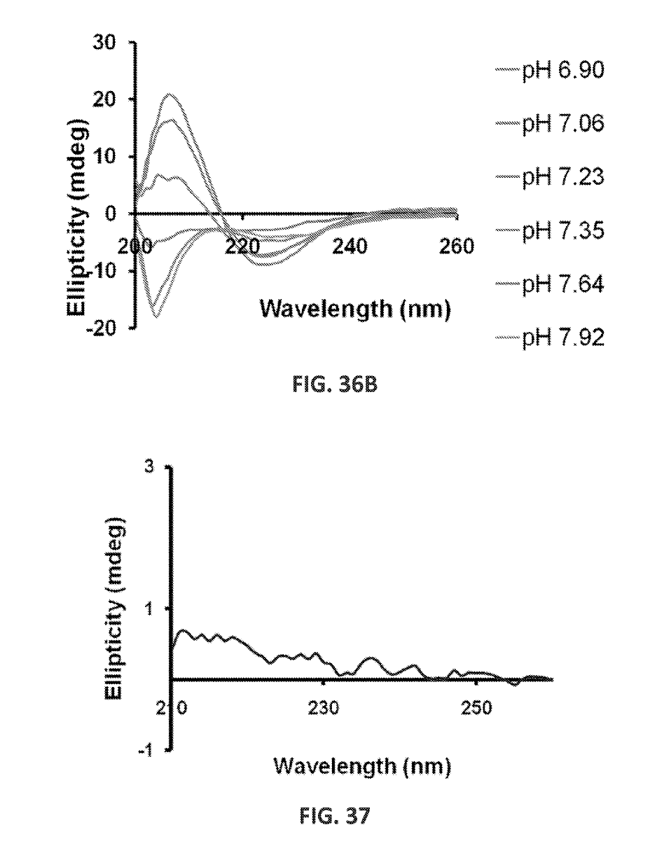

Finally, a medium that accurately replicated the ionic strength and the crowded environment of pure serum and enabled characterization of the PA self assembly behavior using CD and CAC measurements without interference from protein signals was created. This artificial serum solution contains 1.8 mM of 20 kDa PEG, 150 mM NaCl and 2.2 mM CaCl₂. A concentration-pH phase diagram (FIG. 21) was then constructed for pure PA65 in this artificial serum solution using transition points obtained via concentration dependent CD (FIGS. 33A-33B) and pH dependent CAC (FIG. 34) measurements. To evaluate the effectiveness of this simulated solution to mimic real serum, pH dependent CD values were collected for various concentrations of PAmix2 in artificial serum (FIG. 35) and FA was obtained for the same concentrations in pure serum. Just as observed in PAmix1 (FIG. 16), the transition points of the PAmix2 were shifted by ˜0.5 unit to basic pH values relative to the pure PA. Remarkably, the pH transitions in pure and artificial serum agree within ˜0.08 pH units. Hence, this artificial serum precisely mimics real serum conditions and provides a simple platform for predicting self assembly behavior in vivo.

### Example 4: Programming pH-Triggered Self-Assembly Transitions Via Isomerization of Peptide Sequence

In this Example, a general relationship between the position of the β-sheet forming Isoleucine (I) residue in the primary PA sequence and the pH of self-assembly morphology transition is established. To accomplish this, palmitoyl-AIAAEEEE-NH₂ (SEQ ID NO:41 for bolded portion) (PA68), palmitoyl-AAIAEEEE-NH₂ (SEQ ID NO:42 for bolded portion) (PA69), palmitoyl-AAAIEEEE-NH₂ (SEQ ID NO:43 for bolded portion) (PA70), and palmitoyl-AIAAEEEEK(DO3A:Gd)—NH₂ (SEQ ID NO:44 for bolded portion) (PA71) were synthesized and their pH-triggered self-assembly behavior compared to the well characterized control systems, PA1 and PA5 (Table 14). Results show that moving the Isoleucine farther away from the palmitoyl tail preferentially induces nanofiber formation over spherical micelles.

TABLE 14
Synthesized PA molecules

| Molecule | Sequence | SEQ ID NO:* |
|---|---|---|
| PA1 | Palmitoyl-IAAAEEEE-NH₂ | SEQ ID NO: 1 |
| PA68 | Palmitoyl-AIAAEEEE-NH₂ | SEQ ID NO: 41 |
| PA69 | Palmitoyl-AAIAEEEE-NH₂ | SEQ ID NO: 42 |
| PA70 | Palmitoyl-AAAIEEEE-NH₂ | SEQ ID NO: 43 |
| PA5 | Palmitoyl-IAAAEEEEK(DO3A:Gd)-NH₂ | SEQ ID NO: 5 |
| PA71 | Palmitoyl-AIAAEEEEK(DO3A:Gd)-NH₂ | SEQ ID NO: 44 |

*Sequence for bolded portion
PA Structure and Design

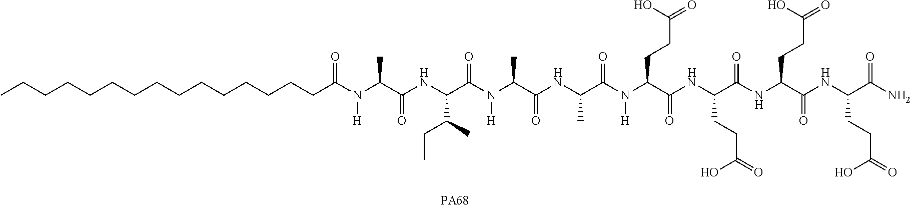 [SMILES: CCCCCCCCCCCCCCCC(=O)N[C@@H](C)C(=O)N[C@H](C(=O)N[C@@H](C)C(=O)N[C@@H](C)C(=O)N[C@@H](CCC(=O)O)C(=O)N[C@@H](CCC(=O)O)C(=O)N[C@@H](CCC(=O)O)C(=O)N[C@@H](CCC(=O)O)C(N)=O)[C@@H](C)CC]

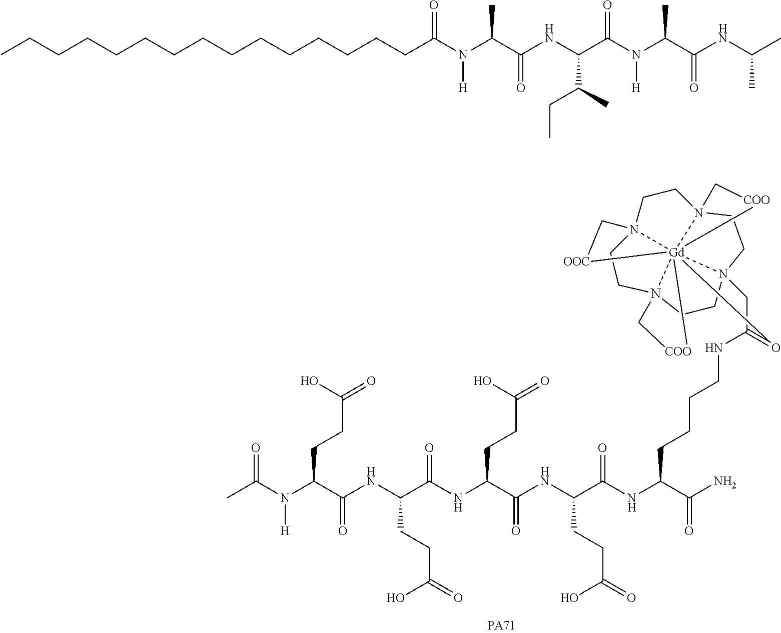

### Experimental Section

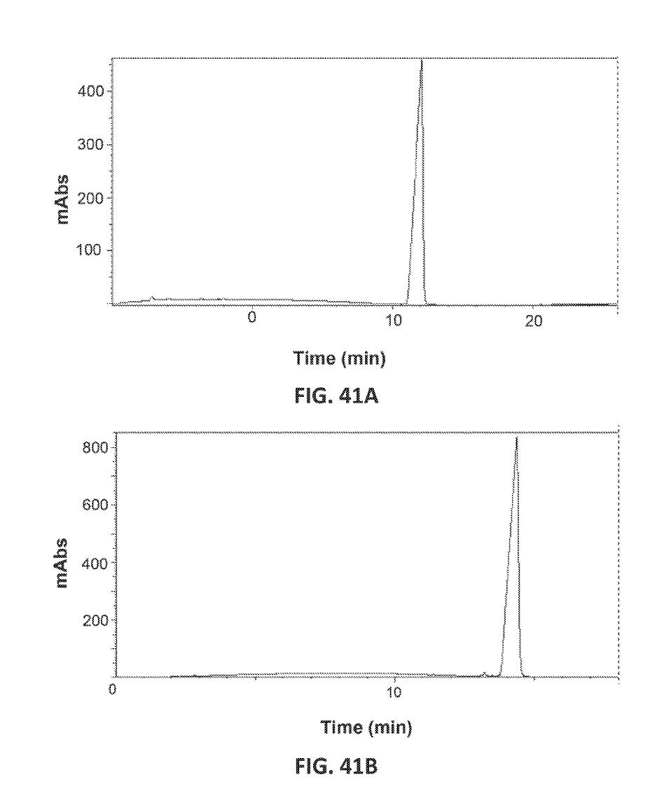

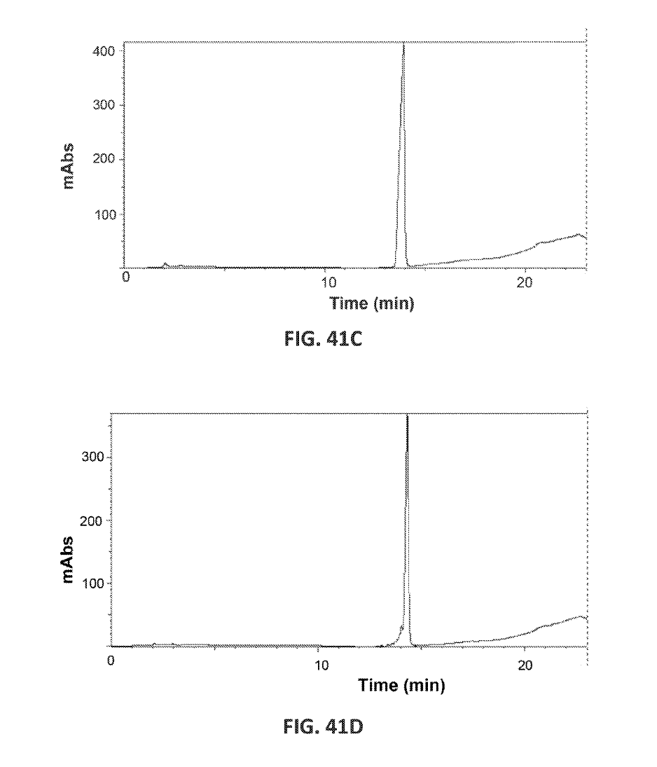

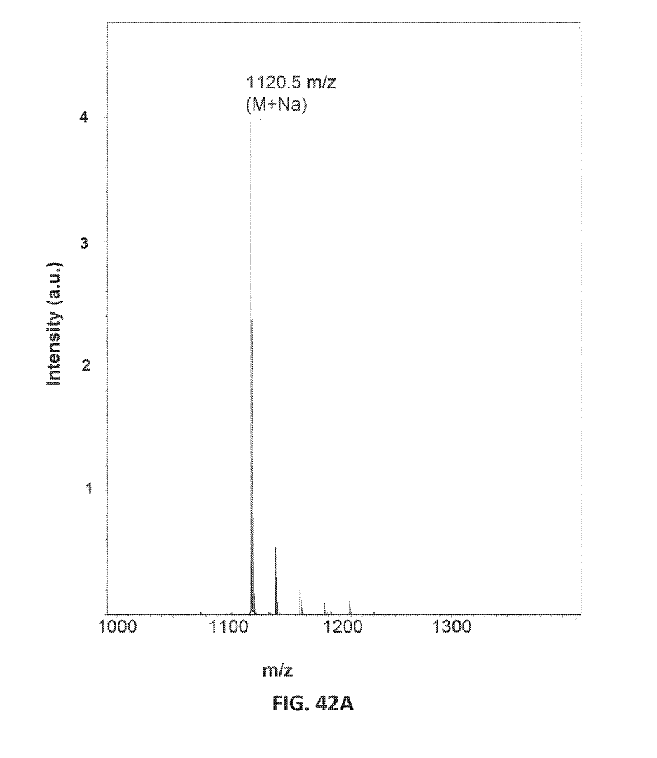

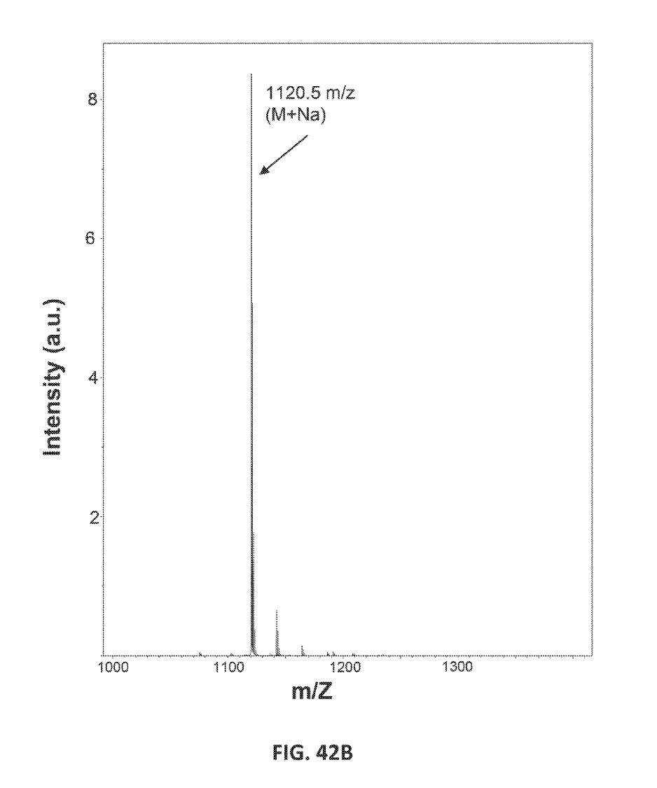

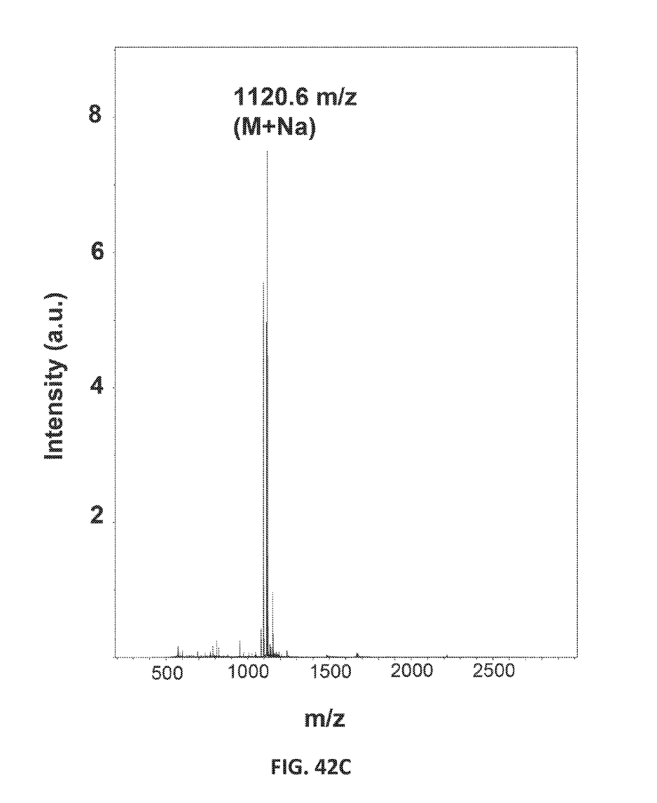

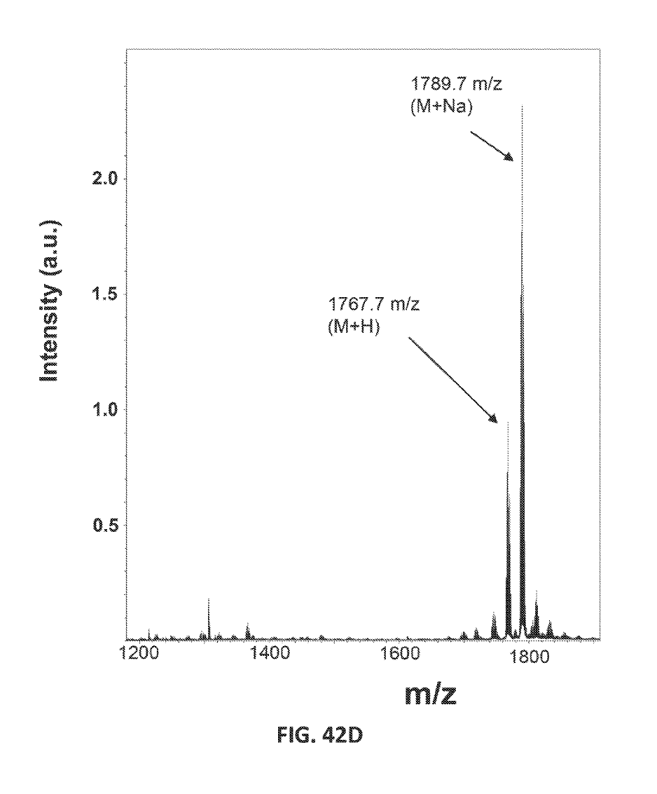

All peptides were synthesized using solid phase Fmoc chemistry, purified via reverse-phase high performance liquid chromatography (HPLC) (FIG. 41) and assessed for purity with Analytical HPLC, electrospray ionization mass spectrometry (FIG. 42) and peptide content analyses. Critical aggregation concentration (CAC) measurements using the pyrene 1:3 method (Aguiar, J., et al. J. Colloid Interface Sci. 2003 258:116-122) were used to determine the concentrations at which aggregates (either micelles or nanofibers) began to form at pH values between 5 and 10. Circular dichroism (CD) spectroscopy was used to determine the pH-dependent secondary structure of PAs at various concentrations. The random coil pattern in CD spectrum corresponds to either single molecules or micelles, and the β-sheet pattern corresponds to the nanofiber morphology (Ghosh, A. et al. J. Am. Chem. Soc. 2012 134:3647-3650). In PAs that undergo pH-triggered transitions between the single molecule/micelle and nanofiber morphologies, the CD transition point was defined as the pH value at which the ellipticity at 205 nm rose from a negative value (random coil) to zero along with the appearance of a negative band near 218 nm. In addition to CAC and CD measurements, conventional transmission electron microscopy (TEM) was used to confirm the PA morphology at different pH values.

Synthesis of Peptide Amphiphiles (PAs):

All amino acids were purchased from AnaSpec Inc. unless otherwise specified. PAs were synthesized using standard solid-phase Fmoc chemistry. All PAs were synthesized using Sieber resin (AAPPTEC). Peptides were made manually on a 0.50 mmol-scale using the following procedure:

The Sieber resin was placed in a shaker vessel and swelled with dichloromethane (DCM) for 30 minutes, then again swelled with dimethyl formamide (DMF) for 30 minutes. A solution of 20% piperidine in DMF was used to remove the Fmoc protecting group on the resin. A Kaiser test was used to confirm the removal of the Fmoc protecting group. To couple amino acids to the deprotected resin or peptide chain, activation of the amino acid using O-Benzotriazole N,N,N′,N′-tetramethyluroniumhexafluorophosphate (HBTU) or, for higher coupling efficiency, 2-(7-Aza-1H-benzotriazole-1-yl)-1, 1,3,3-tetramethyluronium hexafluorophosphate (HATU) was used. Each coupling solution contained 3.95 Eqv. of the amino acid desired, 4 Eqv. of HOBt/HOAt, 4 Eqv. of HBTU/HATU, and 6 Eqv. of Diisopropyl ethylamine (DIPEA) with respect to PA allowing at least 2 hours of coupling per amino acid. A few drops of Triton X-100 (surfactant) were also added to prevent aggregation and increase coupling efficiency. Resin cleavage of the peptide was done by addition of the following solutions: For PA71, a solution containing 1% TFA, 2% Anisole, 1% Triisopropyl silane (TIS) and 95% DCM was used in five 20 mL portions for 20 minutes each. DCM and TFA were removed under vacuum and the PA was precipitated using cold water, and then filtered. For all other PAs, a solution of 95% TFA, 2% Anisole, 1% TIS and 2% water was used, shaken for 3 hours. TFA were removed under vacuum and the PA was precipitated using cold diethyl ether followed by filtration.

Attachment of DO3A:

Following the precipitation of the PA, a 1,4,7,10-tetraazacyclododecane-1,4,7-trisacetic acid (DO3A) chelate was conjugated to the ε-NH₂ of the lysine side chain using solution phase coupling. The PA was added to pyridine and stirred until dissolved at 60° C. The solution was then allowed to stir at room temperature. A coupling solution containing ˜5 ml pyridine, 2 Eqv. HATU, 4.4 Eqv. of DIPEA, and 2 Eqv. of DO3A (relative to the PA) was prepared. This solution was added to the PA and the combination was stirred overnight. Excess pyridine was then removed under vacuum and the peptide was crashed out using cold water, and then filtered. The resulting solid was then stirred in a cocktail containing 95% TFA, 2% Millipore water, 2% anisole, and 1% TIS to remove the protecting tert-butyl groups. The deprotection cocktail was stirred at room temperature for 24 hours. Excess TFA was removed under vacuum and the PA was precipitated out of solution using cold diethyl ether followed by filtration.

Purification of PAs:

The crude PA was dissolved in a 10% acetonitrile aqueous solution with a few drops of NH4OH added to aid in solubility. Following dissolution, the PA solution was filtered using a 0.45 μm polytetrafluoroethylene (PTFE) filter. Purification was performed using a Shimadzu preparative high performance liquid chromatography (HPLC) system (dual pump system controlled by LC-MS solution software) with an Agilent PLRP-S polymer column (Model No. PL12123100 150 mm×25 mm) under basic conditions. The product was eluted using a linear gradient from 10% acetonitrile to 20% acetonitrile over 22.5 minutes, followed by a linear gradient from 20% acetonitrile to 40% acetonitrile over an additional 67.5 minutes, both containing 0.1% NH₄OH (v/v). The presence of the desired product in collected fraction was confirmed using electrospray ionization time-of-flight mass spectrometry (ESI-TOF MS) (Bruker). The purity of product-containing samples was assayed using a Shimadzu analytical HPLC system. Samples of high purity (>90%) were combined. Pure fractions were relieved of acetonitrile under vacuum and freeze-dried to produce a white, fluffy powder.

Incorporation of Gd3+ into PA:

A small amount (˜15 mg) of the purified and fully deprotected PA71 was dissolved in water with 2 equivalents of GdCl₃ in 0.01 M HCl. The solution was raised to exactly 5.0 pH using small amounts of 0.05 M NaOH and stirred at 60° C. for 10 minutes. The reaction was brought back to room temperature and the pH was again adjusted to pH 5.0 since the reaction results in a lowering of pH. The reaction was returned to 60° C. and stirred overnight. The solution was then returned to room temperature and the pH was adjusted to 10 or greater using 0.1 M NaOH to precipitate excess Gd³⁺ in the form of Gd(OH)₃. The solution was then filtered using a 0.45 μm PTFE filter and the pH was adjusted to neutral using 0.1 M HCl. The solution was then dialyzed using a SpectraPor Biotech cellulose ester dialysis membrane with 100-500 D MW cut-off against Millipore water. The buffer water was changed 4 times over a 48 hours period. The dialyzed solution was then freeze-dried to yield a fluffy white powder of pure PA71.

Circular Dichroism (CD) Spectroscopy:

Measurements were conducted using a Jasco-815 CD spectrometer. Each CD trace is the average of 3 accumulations and baseline subtracted using an aqueous solution containing only 150 mM NaCl and 2.2 mM CaCl₂. PA stock solutions were prepared at a basic pH containing 1.00 mM PA and no salts. Samples containing various concentrations of the aqueous PAs with 150 mM NaCl and 2.2 mM CaCl₂ were prepared and heated at 80° C. for 30 minutes at 9 or greater pH. After heating, samples were allowed to cool to room temperature and pH was adjusted as desired for CD measurements.

Critical Aggregation Concentration (CA C) Determination Using the Pyrene 1:3 Method:

The pyrene 1:3 method was used to determine CAC for the PAs (Aguiar, J., et al. J. Colloid Interface Sci. 2003 258:116-122). For both PA71 and PA68, CAC measurements were performed at various pH values (5-10). Samples of high PA concentration (300-750 μM) were prepared in deionized water containing 150 mM NaCl and 2.2 mM CaCl₂. These samples were raised to 9 or greater pH using NaOH and heated at 80° C. for 30 minutes. Samples were then allowed to cool to room temperature and pH was altered to the desired value using dilute NaOH and HCl. Solutions containing only aqueous 150 mM NaCl and 2.2 mM CaCl₂ and solutions containing these salts, a 1:1 solution of water and methanol, and 62 μM pyrene were also prepared. Each of these solutions was divided into various portions and adjusted to appropriate pH values. The PA solutions were serially diluted with the corresponding salt solutions with a total of 125 μL at each concentration. 5 μL of the pyrene containing solution as added to each dilution for a final pyrene concentration of 2.4 μM. Each diluted sample was probed for fluorescence with an excitation wavelength of 335 nm. Fluorescence was recorded with particular attention paid to the 376 nm and 392 nm emission peaks. The ratio of these two peaks was plotted versus PA concentration on a log scale. The CAC was determined from an abrupt change in the slope of the plot using the least-squares fitting technique.

Peptide Content Analysis:

Peptide content analysis was performed on lyophilized samples by the UC Davis Molecular Structure Facility to verify the amino acid stoichiometry and determine the residual salt concentration for all peptides. The mg of total peptide amphiphile/mg of solid is listed in the table below. All further CAC and CD measurements were scaled by these factors to determine the true concentration.

TABLE 15
Peptide Content

| PA | mg PA/mg Solid |
|---|---|
| PA1 | 88% |
| PA68 | 56% |
| PA69 | 63% |
| PA70 | 73% |
| PA5 | 96% |

Transmission Electron Microscopy (TEM):

TEM images were obtained at various concentrations and pH values, with 150 mM NaCl and 2.2 mM CaCl₂ in deionized water. Solutions were heated at 80° C. for 30 minutes and gradually cooled to room temperature. Following temperature equilibration, pH adjustment was achieved using HCl or NaOH. 5 μL of this solution was pipetted onto a Carbon Formvar grid (Electron Microscopy Sciences) and allowed to sit for 2 minutes before being wicked dry using filter paper strips. The samples were negatively stained with 1 wt % uranyl acetate and imaged under a FEI Tecnai G2 Bio TWIN TEM system operating at 100 kV.

Results

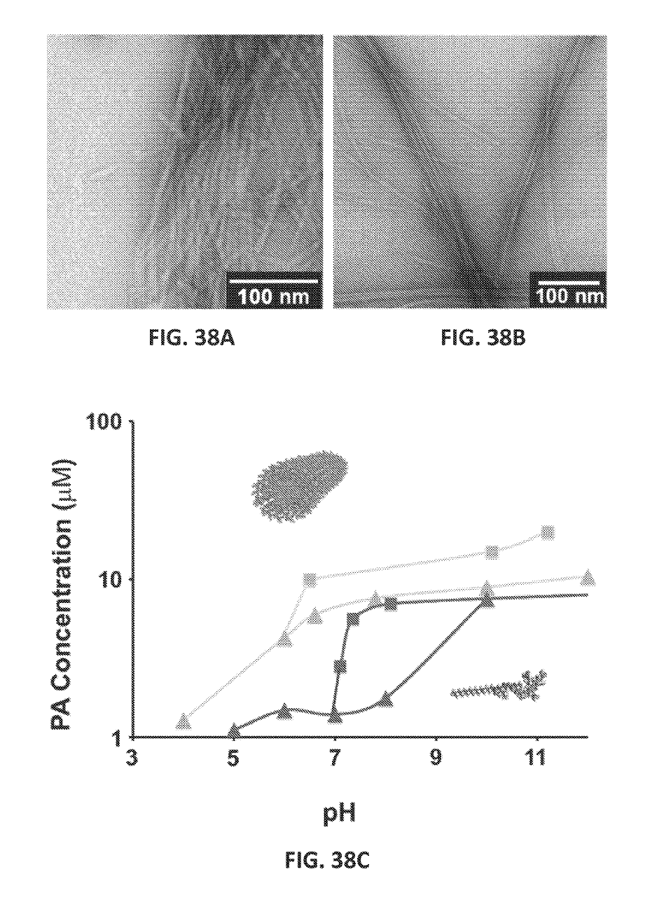

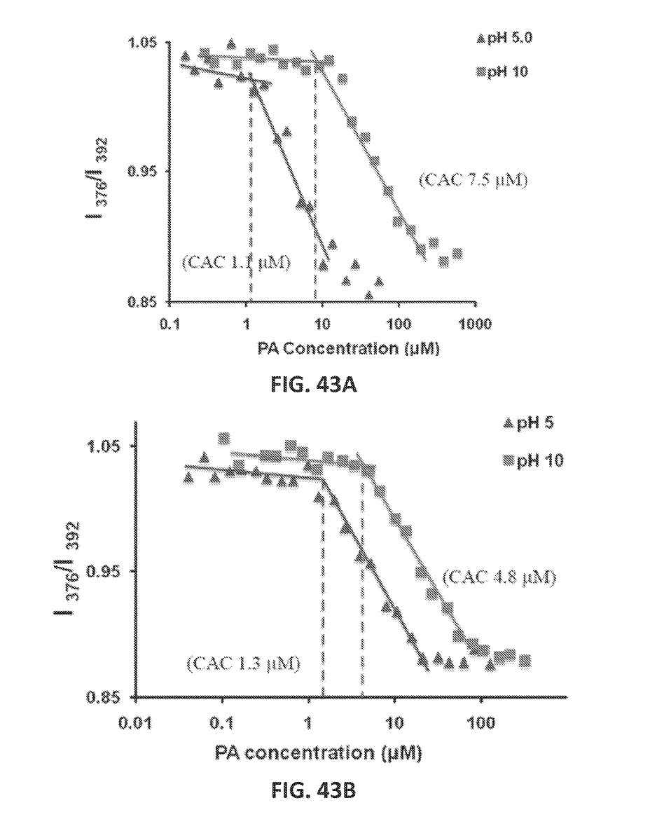

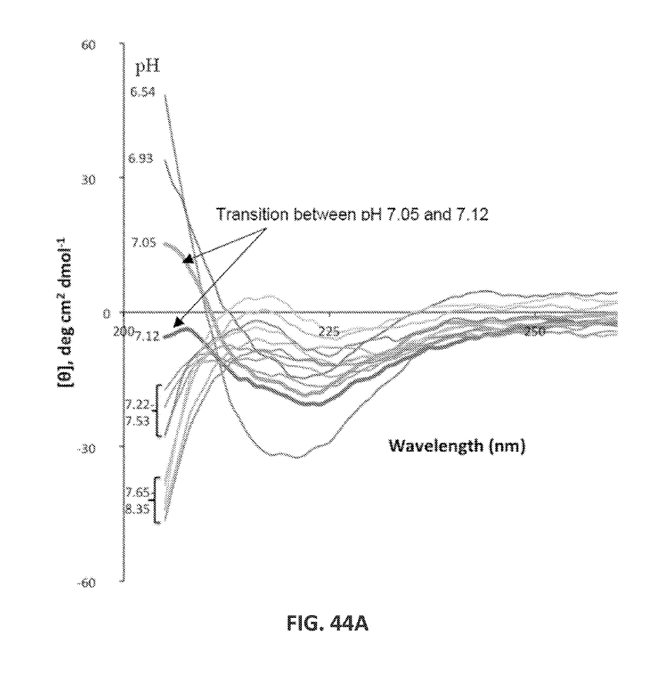

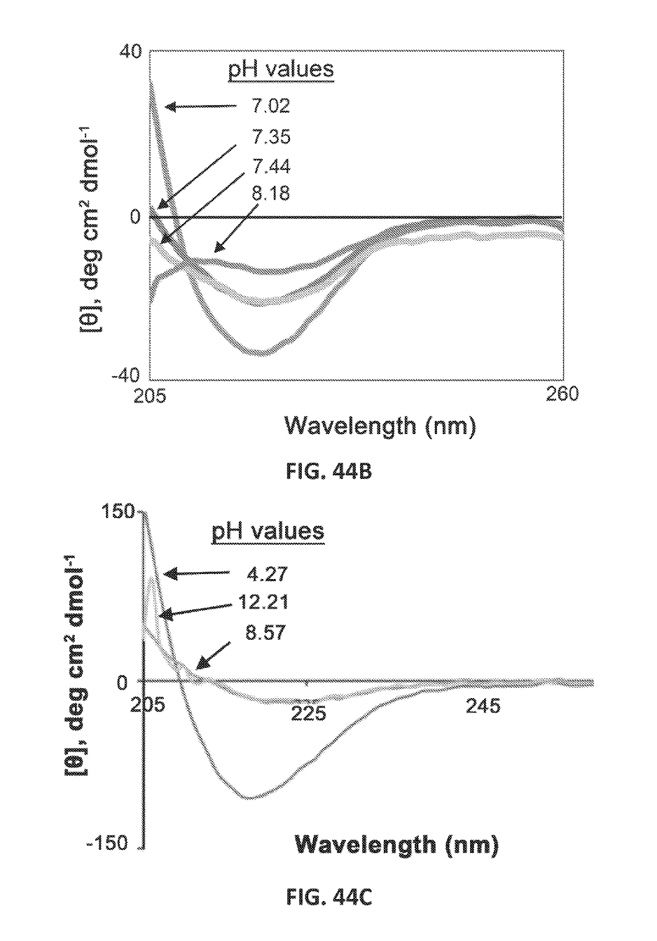

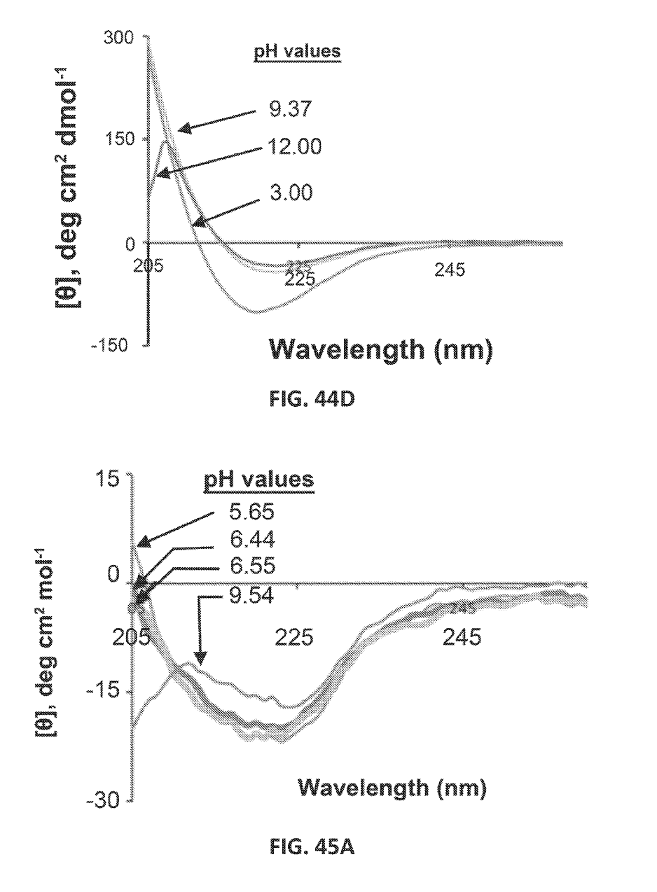

As shown in the Examples above, concentration-pH phase diagrams for the control molecules PA1 and PA5 were established in simulated serum salt solutions containing 150 mM NaCl and 2.2 mM CaCl₂ (Ghosh, A. et al. J. Am. Chem. Soc. 2012 134:3647-3650). In order to understand the effects of sequence isomerization on pH-triggered self-assembly, concentration-pH phase diagrams were mapped out for PAs in which the Isoleucine was shifted away from the palmitoyl tail by one residue (PA68 and PA71). The single molecule to micelle/nanofiber transitions at various pH values were determined using CAC measurements, and the pH-dependent transitions from either spherical micelles or isolated molecules into nanofibers at various PA concentrations were determined via CD spectroscopy, in the same salt solution. The phase diagram of PA68 is overlaid with PA1 (FIG. 38C). The CAC values for PA68 are pH-dependent, ranging from 1.1 μM to 7.5 μM at pH 5 and 10, respectively (FIG. 43A). Both PA1 and PA68 show an increase in CAC values in more basic conditions due to the greater electrostatic repulsion among deprotonated glutamic acids. However, the CACs for PA68 are 3-5 times lower than those for PA1 at comparable pH values. This indicates an increase in the attractive forces in these surfactant-like molecules when the Isoleucine is moved by one residue away from the tail. CD spectra of PA68 confirmed that the nanofibers were formed at lower concentrations relative to PA1 (FIG. 44). At 8.4 μM and above, PA68 formed nanofibers across all pH values. TEM images collected for 100 M PA68 at pH 5.0 and 8.3 (FIG. 38A. 38B) confirmed the nanofiber morphology. The PA68 nanofiber diameter 9.5±0.8 nm is close to the observed diameters for PA1 nanofibers (9.1±1.5 nm) from previous TEM measurements. Therefore, the self-assembled morphology does not significantly change upon changing the position of the Isoleucine in the hydrophobic region.

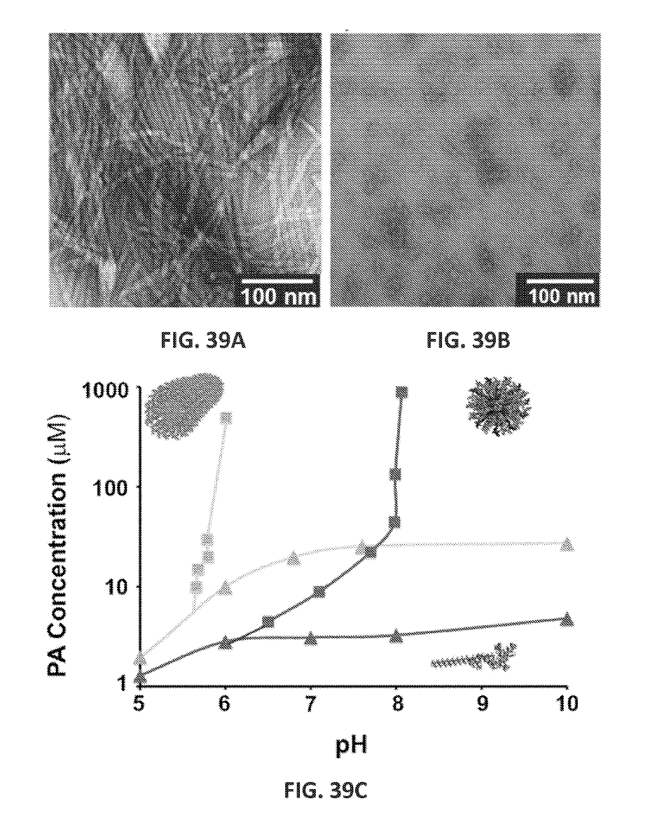

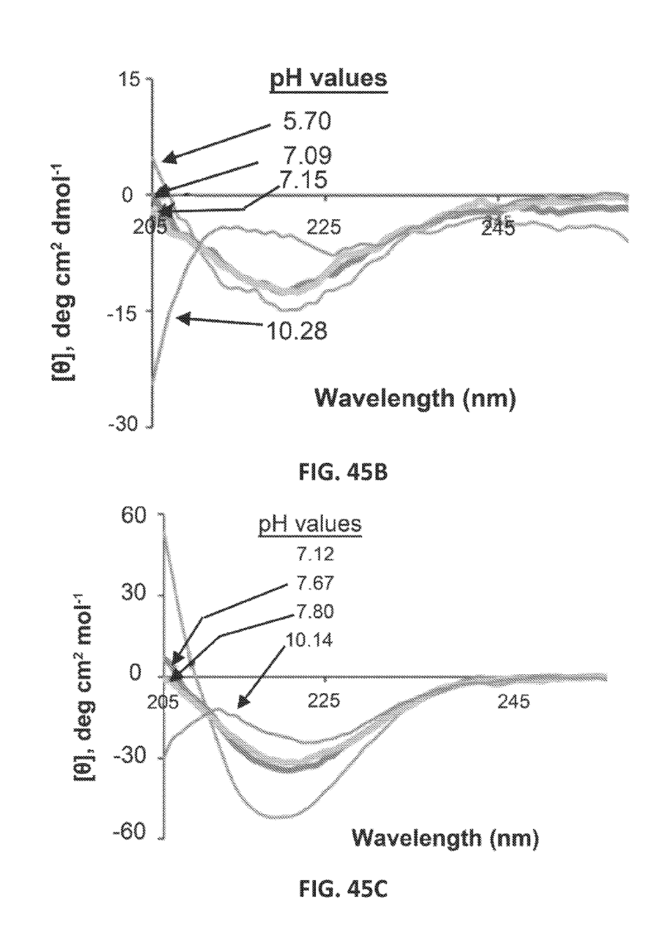

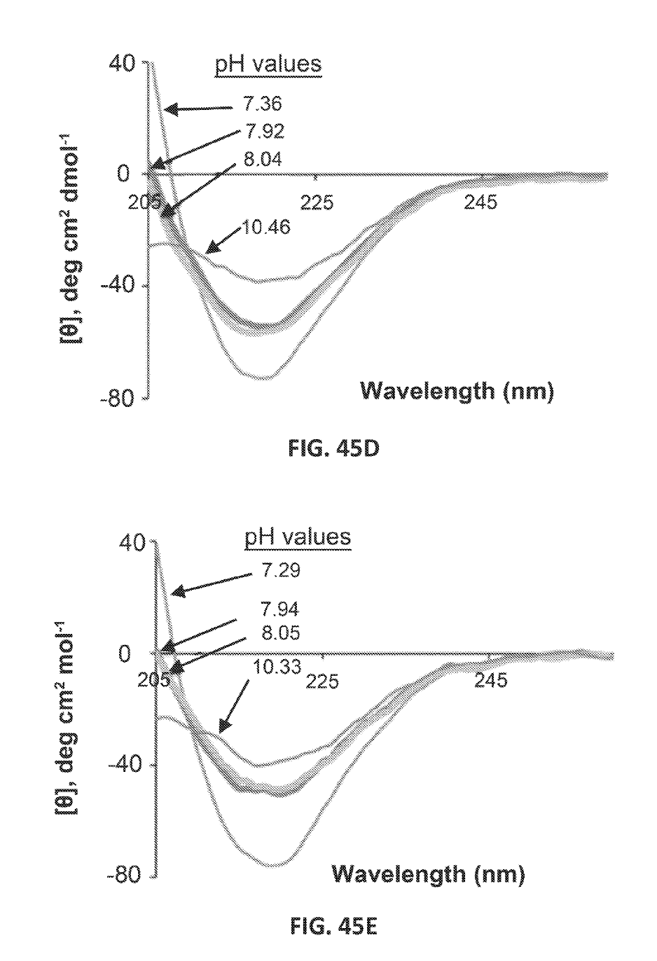

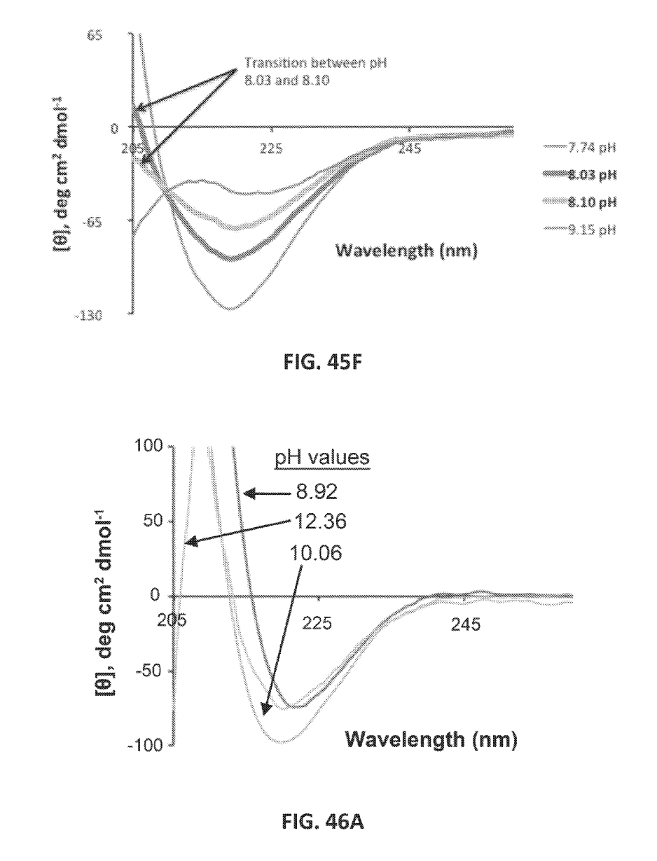

The lowering of the CAC values upon moving the Isoleucine away from the palmitoyl tail was also observed in PAs containing a sterically bulky Gd:DO3A moiety. The phase diagram of PA71 was measured and overlaid with the previously characterized PA5 (FIG. 39C). CAC values for PA71 are also pH-dependent, ranging from 1.3 to 4.8 μM at pH 5 and 10, respectively (FIG. 43B). Furthermore, the CAC values for PA71 are consistently less than those for PA5. For example, PA71 exhibits over a 6-fold lower CAC value than PA5 at pH ˜7. This indicates that PA71 has greater attractive supramolecular interactions relative to PA5. The sterically bulky Gd:DO3A moiety induces a spherical micelle morphology at high pH values and high concentrations. Moving the Isoleucine away from the palmitoyl tail shifts the transition pH to more basic values. For PA71, the transition pH between spherical micelles and nanofibers was concentration dependent below 45 μM. The transition pH shifted to more acidic values (from 7.7 to 6.5) with lower PA concentrations (FIG. 45). Above 45 μM, the pH of transition does not change with PA concentration, similar to PA5. At all concentrations, PA71 exhibited a transition pH that was more basic by 1.5 to 2.0 pH units relative to PA5. Again, these morphologies were verified with TEM measurements (FIGS. 39A, 39C). At pH 6.6 (150 μM), nanofibers of ˜11.3±1.1 nm width and in excess of 1 μm length were identified, often in bundles. At pH 10.0 (1 mM) PA71 showed self-assembled spheres that were ˜1 0.1±0.9 nm diameter. The diameters of both morphologies were comparable to previous TEM observations for PA5, indicating that isomerization has a negligible effect on the size of the self-assembled structures (Ghosh, A. et al. J. Am. Chem. Soc. 2012 134:3647-3650). This shift in transition pH to more basic values reflects the increased 1-sheet propensity of the Isoleucine when moved one amino acid away from the palmitoyl tail.

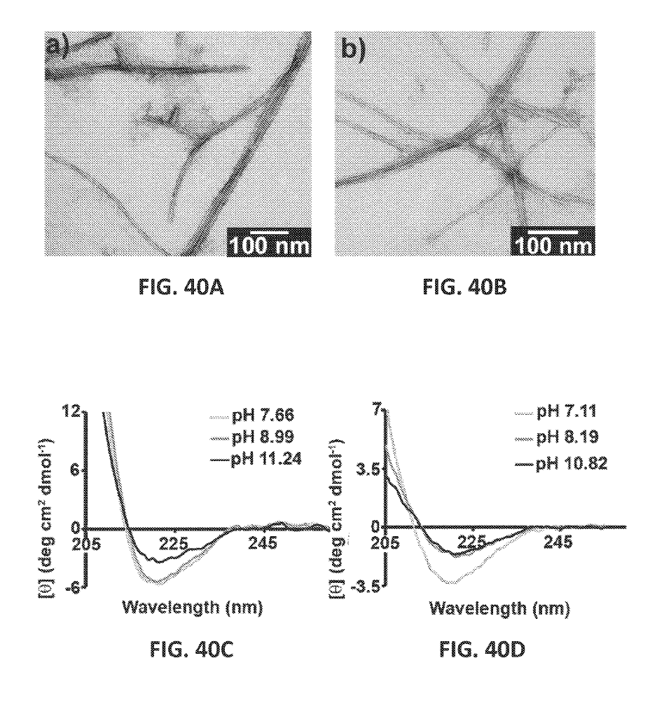

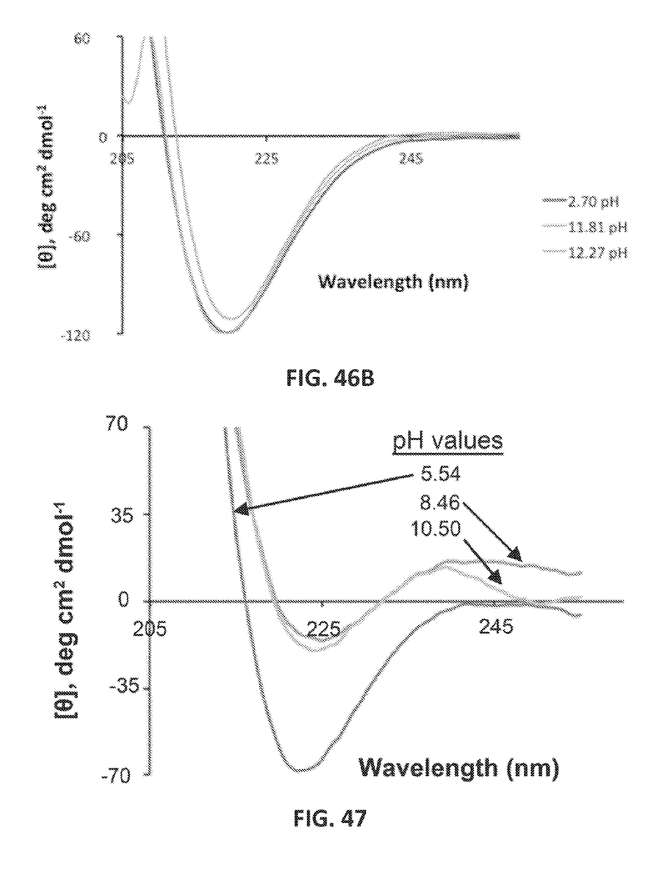

Moving the Isoleucine to either the third or fourth amino acid position from the palmitoyl tail induces nanofiber formation across pH 5-10 and all detectable concentration values. CAC values for PA71 were below the detectable limit (˜200-300 nM) at all pH values tested except at pH 10 (1.3 μM). PA70 was observed to be in the aggregated state across all pH and concentration values and CAC values could not be detected via the pyrene 1:3 method. CD spectra for PA71 revealed exclusively 3-sheet formation irrespective of concentration and pH (FIGS. 40C, 46). The nanofiber morphology was confirmed at pH 8.0 via TEM measurements (FIG. 40A). The observed fibers were >1 μm in length and -9.6±0.5 nm in diameter. CD spectra for PA70 also exhibited a β-sheet secondary structure regardless of concentration and pH (FIG. 40D, FIG. 47). TEM images collected at pH 8.0 of PA70 additionally show the formation of nanofibers with lengths >1 μm and -9.0±0.8 nm in diameter (FIG. 40B).

Together, these results clearly demonstrate that simply moving the position of a single β-sheet forming residue in a PA can dramatically alter the pH- and concentration-dependent self-assembly behavior. More specifically, moving the Isoleucine farther away from the palmitoyl tail enhances its ability to promote β-sheet formation, inducing the nanofiber morphology at lower concentrations and more basic pH values. The effect of isomerizing the peptide sequence is much more significant than that of changing the identity of the amino acid. In our previous studies, substituting the Isoleucine of PA1 with amino acids having weaker β-sheet propensity such as Valine, and Tyrosine, shifted the transition only by 0.4-0.6 pH units (Ghosh, A. et al. J. Am. Chem. Soc. 2012 134:3647-3650). Here, moving the Isoleucine to the second amino acid position from the palmitoyl tail, shifts the transition pH by 1.5-2.0 units, and in the third and fourth position, nanofibers form regardless of pH at all observable concentrations.

When positioned next to the palmitoyl acid tail, hydrophobic residues like Isoleucine can readily associate with the hydrophobic alkyl tail, reducing their preference to form a β-sheet secondary structure. This association interrupts n-sheet formation among neighboring PA molecules. Creating distance between the Isoleucine and the hydrophobic core via isomerization makes their hydrophobic association less favorable. Instead, the Isoleucine facilitates hydrogen-bonding interactions among neighboring PAs, thus adding to the attractive interactions of the hydrophobic core. Another possibility is that when placed in close proximity to the Glutamic acid residues, the hydrophobic Isoleucine could reduce the electrostatic repulsive interactions of these charged anionic residues. It is well established that the pKa of an anionic amino acid shifts to more basic values when placed in a more hydrophobic environment. These effects combined result in the PA adopting nanofiber morphologies at lower concentrations and at more basic pH values. This preference in forming nanofibers becomes even more pronounced as the distance between the hydrophobic residue and the palmitoyl core increases in PA69 and PA70. Indeed, previous studies using coarse-grained molecular dynamics simulations suggest that the hydrophobic amino acid positioned next to the palmitoyl tail strongly associates with the alkyl tail core in another peptide amphiphile system, palmitoyl-VVVAAAEEE-NH₂ (PA72) (SEQ ID NO:45 for bolded portion) (Fu, I. W., et al. Adv. Healthcare Mater. 2013 2:1388-1400). These simulations also suggest that the third and fourth amino acid residue away from the palmitoyl tail have the highest probability of forming a hydrogen bond with a neighboring peptide, thus promoting the β-sheet secondary structure (Fu, I. W., et al. Adv. Healthcare Mater. 2013 2:1388-1400). These simulations are consistent with our observation that moving the hydrophobic residue to the third and fourth position maximizes its β-sheet propensity exclusively forming the nanofiber morphology, irrespective of pH and concentration.

### Example 5: Relationship Between the Alkyl Chain Length, Number of Strongly Hydrophobic Amino Acids, the Number of Anionic Amino Acids in the PA Sequence, and the pH of the Self-Assembly Morphology Transition

This Example, a general relationship between the alkyl chain length, number of strongly hydrophobic amino acids, and the number of anionic amino acids in the PA sequence, and the pH of the self-assembly morphology transition are established. This was accomplished by synthesizing and comparing the pH-triggered self-assembly behavior of palmitoyl-YYAAEEEEEK(DO3A:Gd)—NH₂ (PA74) (SEQ ID NO:26 for bolded portion) and pentadecyl-YYAAEEEEK(DO3A:Gd)—NH₂ (PA75) (SEQ ID NO:45 for bolded portion) to palmitoyl-YYAAEEEEK(DO3A:Gd)—NH₂ (PA73) (SEQ ID NO:45 for bolded portion), as well as, palmitoyl-YAAAEEEEEK(DO3A:Gd)—NH₂ (PA76) (SEQ ID NO:46 for bolded portion) and pentadecyl-YAAAEEEEK(DO3A:Gd)—NH₂(PA77) (SEQ ID NO:7 for bolded portion) to palmitoyl-YAAAEEEEK(DO3A:Gd)—NH₂ (PA9) (SEQ ID NO:7 for bolded portion). Results show that either decreasing the attractive forces by removing a —CH₂— unit, or increasing the repulsive forces by adding a glutamic acid produce similar changes in the pH-concentration self-assembly behavior, and can counterbalance the additional propensity for nanofiber formation with an additional strongly hydrophobic tyrosine residue. Controlling the pH- and concentration-trigger of self-assembly is a matter of balancing the different attractive and repulsive forces.

TABLE 16
Synthesized PA molecules

| Molecule | Sequence | SEQ ID NO:* |
|---|---|---|
| PA73 | palmitoyl-YYAAEEEEK(DO3A:Gd)-NH₂ | SEQ ID NO: 45 |
| PA74 | palmitoyl-YYAAEEEEEK(DO3A:Gd)-NH₂ | SEQ ID NO: 26 |
| PA75 | pentadecyl-YYAAEEEEK(DO3A:Gd)-NH₂ | SEQ ID NO: 45 |
| PA9 | palmitoyl-YAAAEEEEK(DO3A:Gd)-NH₂ | SEQ ID NO: 7 |
| PA76 | palmitoyl-YAAAEEEEEK(DO3A:Gd)-NH₂ | SEQ ID NO: 46 |
| PA77 | pentadecyl-YAAAEEEEK(DO3A:Gd)-NH₂ | SEQ ID NO: 7 |

*Sequence for bolded portion
Structure of PA75

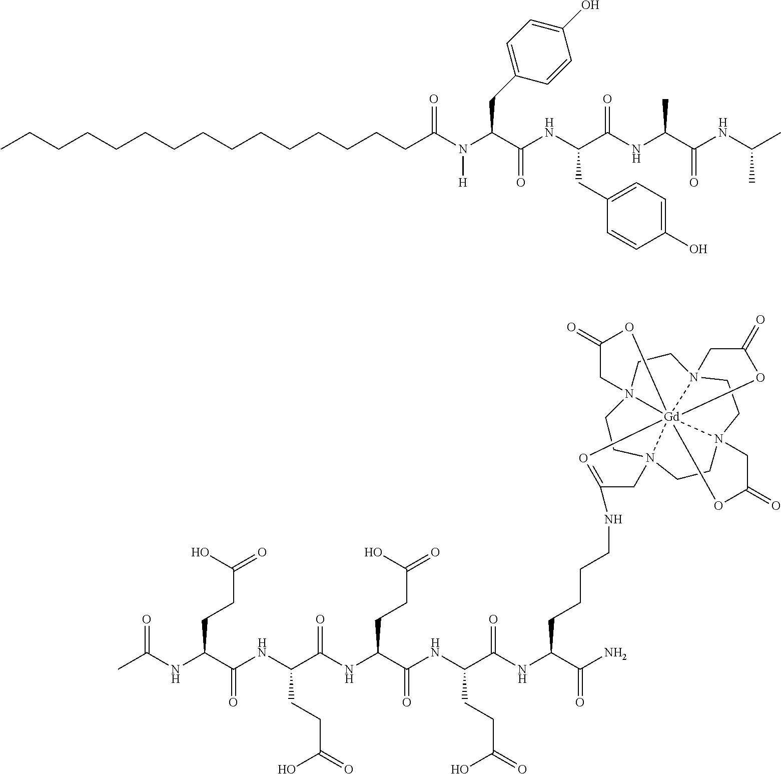

### EXPERIMENTAL

Chemicals and Reagents

All PAs were prepared using solid phase Fmoc peptide synthesis with amino acids purchased from Anaspec Inc, and purified via reverse-phase high-performance liquid chromatography (HPLC). Analytical HPLC and electrospray ionization mass spectrometry (ESI-MS) were used to assess the purity of the PA.

Circular Dichroism

Circular Dichroism (CD) was used to determine the pH-dependent morphology transition of the PAs. A random coil pattern on the CD spectrum represents a micelle or single molecule, while a β-sheet pattern corresponds to molecules that are assembled into nanofibers. A β-sheet CD curve has a minimum at 218-220 nm. The pH at which the micelle/single molecule to nanofiber transition occurs was defined as the midpoint pH between the lowest pH random coil pattern (no minimum at 218-220 nm) and the highest pH β-sheet curve. All samples were basified to a pH of 10, stirred at 90° C. for 30 minutes in an oil bath and cooled to room temperature in order to ensure the molecule was not trapped in a thermodynamically stable state after lyophilization. CD measurements were carried out on a JASCO J-815 Spectrometer using a 0.5 or 1 cm path length quartz cuvette with concentrations of the PA ranging from 10 μM to 500 μM in a isotonic salt solutions consisting of 150 mM NaCl and 2.2 mM CaCl₂. Aqueous HCl and NaOH solutions were added to adjust the pH of the PA solution and an Accumet XL15 pH meter (Fisher Scientific), along with an Orion Ross Ultra semimicro electrode (8103BNUWP, Thermo Scientific) were used to accurately set the solution to a desired pH value. Three accumulation were measured at a wavelength range of 260-190 nm at a scanning speed of 100 nm/min with an integration time of 2 or 4 seconds for each data series.

Critical Aggregation Concentration Measurements

Critical aggregation concentration (CAC) measurements were carried out to determine the concentration at which micelles or nanofibers begin to form by using the pyrene 1:3 method, (citation) All samples were basified to a pH of 10, stirred at 90° C. for 30 minutes in an oil bath and cooled to room temperature. HCl and NaOH were then added to set the PA solution specific pH value. PAs with concentrations ranging from 100 μM to 500 μM were serial diluted with 150 mM NaCl and 2.2 mM CaCl₂. A pyrene solution was then added to each of the diluted samples, for a final pyrene concentration of 4.5 μM. 100 μL of each serial diluted sample were transferred into a 96-well plate and the fluorescence emission of the pyrene was monitored by a hybrid reader fluorimeter (BioTek Synergy H4) at room temperature with an excitation wavelength of 335 nm. The pyrene fluorescence was monitored from 360 nm to 430 nm, and the emission peaks at 376 nm and 392 nm were compared at different concentration values to determine the CAC at a specific pH value. These concentrations were determined at pH values ranging from 5-9.

Transmission Electron Microscopy (TEM)

Transmission electron microscopy was employed to confirm the self-assembled morphology at different pH values and concentrations. The sample to be tested was basified to a pH of 10, stirred at 90° C. in an oil bath for 30 minutes and cooled to room temperature. The pH was then adjusted with HCl and NaOH to set the peptide solution (in 150 mM NaCl and 2.2 mM CaCl₂) to a pH of either 5 or 9. Once the desired pH was reached, 5 μL of the sample was placed and spread onto a Carbon Formvar grid (Electron Microscopy Sciences) and allowed to sit for one minute before being wicked dry with filter paper. A 1 wt % uranyl acetate solution was added and spread over the grid to negatively stain the sample, and was wicked dry immediately after application. The grid was then examined by a FEI Tecnai G2 Biotwin TEM at 100 keV. The diameters of each self-assembled morphology was determined via averaging at least 30 different objects.

Results

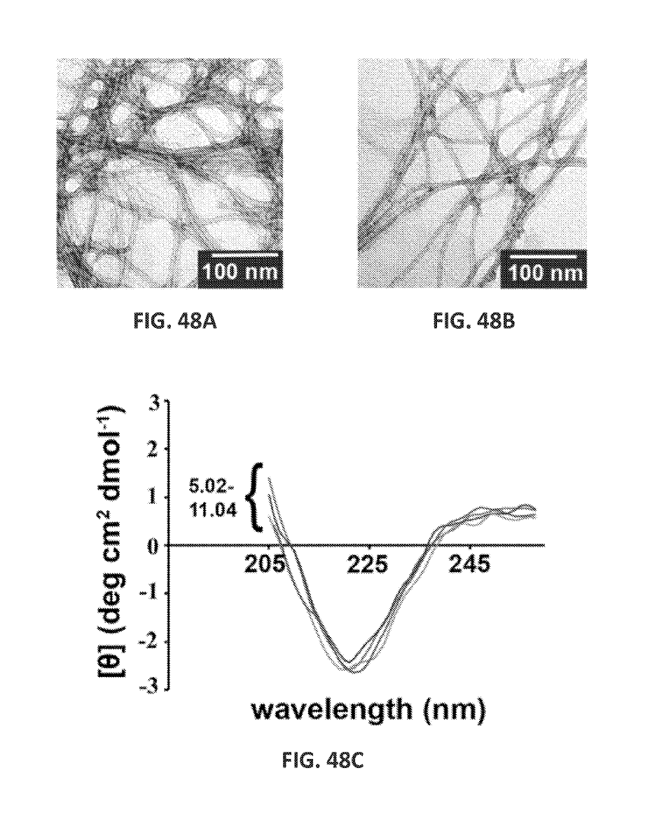

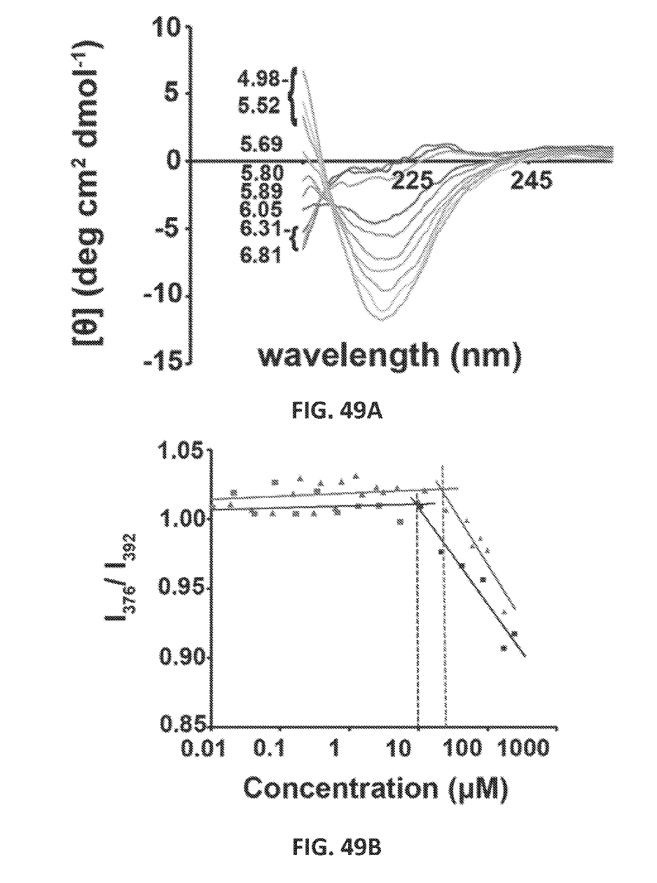

PA73, which features a palmitoyl alkyl tail, two n-sheet forming tyrosine residues, and four glutamic acids, has the strongest attractive forces and weakest repulsive forces of the PAs in this study. The CD of PA73 at 10 μM for pH values ranging from 5-11 reveals a β-sheet pattern, indicative of nanofiber formation (FIG. 48A). The nanofiber morphology was confirmed by TEM of samples prepared at pH values of 5 and 9 of PA73 at 500 μM (FIG. 48B, 48C). The diameters of the nanofibers at pH 5 and 9 were observed to be 8.5±1.0 nm and 8.6±0.9 nm, respectively, with no statistically significant difference as expected. In order to induce a micelle-to-nanofiber transition in this pH range, PA74, having increased repulsive forces via the addition of a fifth glutamic acid, and PA75, having decreased attractive forces through the shortening of an alkyl chain length, were synthesized. FIG. 49A shows the CD of PA74 at 50 μM, which undergoes a pH-dependent morphology transition from an isolated molecules/micelles at basic pH values to nanofibers below a pH of 5.75. To distinguish if PA74 exists as isolated molecules or is assembled into micelles, the CAC was determined at different pH values (FIG. 49B). For instance, the CAC values at pH 7.06 and pH of 9.02 were extrapolated to be 9.3 and 21.6 μM, respectively. The increase in CAC at basic pH values is expected, as the greater degree of deprotonation of the glutamic acid residues leads to enhanced electrostatic repulsion. The pH-dependent CD and concentration dependent CAC was also collected for PA75. Also for PA75, both a pH-dependent morphological transition from isolated molecules/micelles at basic pH values to nanofibers at acidic pH values and an increase in CAC at more basic pH values is observed.

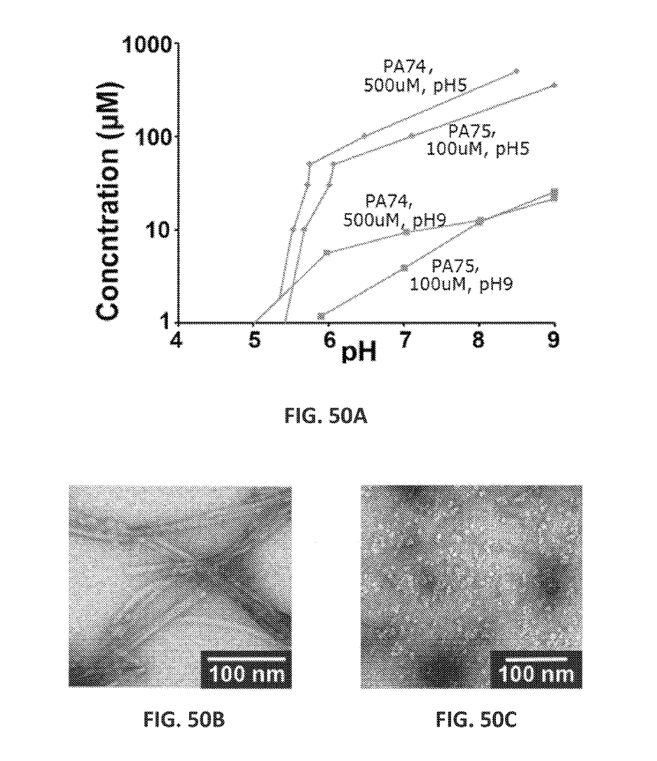

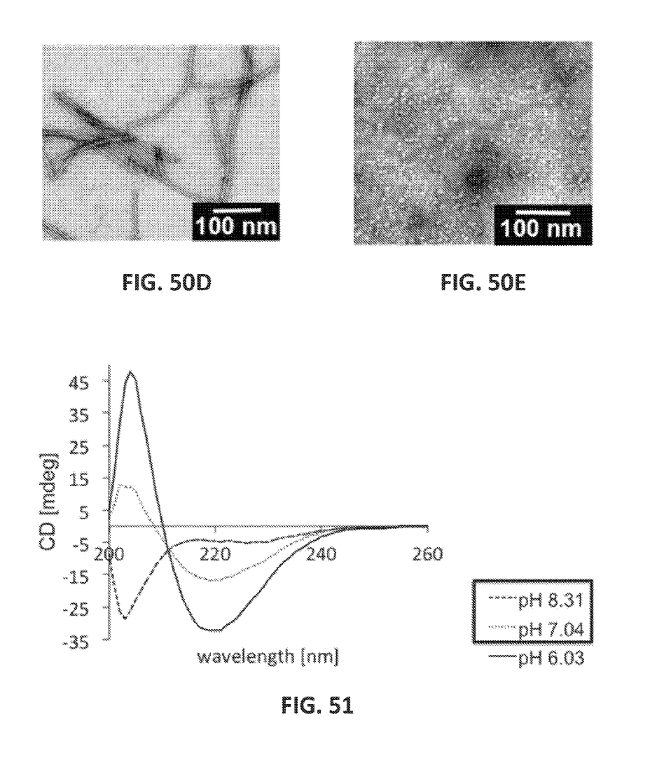

In order to distinguish whether adding a fifth glutamic acid or reducing the alkyl chain length had a greater effect on disrupting nanofiber formation, the concentration-dependent CD and pH-dependent CAC measurements were combined to construct the pH vs. concentration self-assembly phase diagrams for both PA74 and PA75 (FIG. 50A). The self-assembly phase diagrams for PA74 and PA75 were relatively similar, although, PA16 had a greater propensity to form nanofibers at a given concentration than PA75. At all concentrations observed PA75 transitions from micelles to nanofibers at only 0.15-0.63 pH units more basic than PA74. Additionally, at all pH values except at pH 9, the CAC measurements for PA74 were typically higher than PA75 by nearly 500% at pH 6 while only 5% at pH 8. This shows that the increasing repulsive forces by the addition of a glutamic acid has a slightly greater effect on disrupting self-assembly than decreasing the attractive forces by shortening the alkyl tail by one methylene unit. TEM confirm the micelle and nanofiber morphology of PA74 and PA75 at various concentrations and pH values (FIG. 50B-50E). The diameters of the nanofibers for PA74 and PA75 were observed to be 9.7+/−1.2 nm and 8.1±1.3 nm, respectively.

### Example 6: PEGylated Vehicles that Transition at a pH of 6.6-7.4 in Artificial Serum

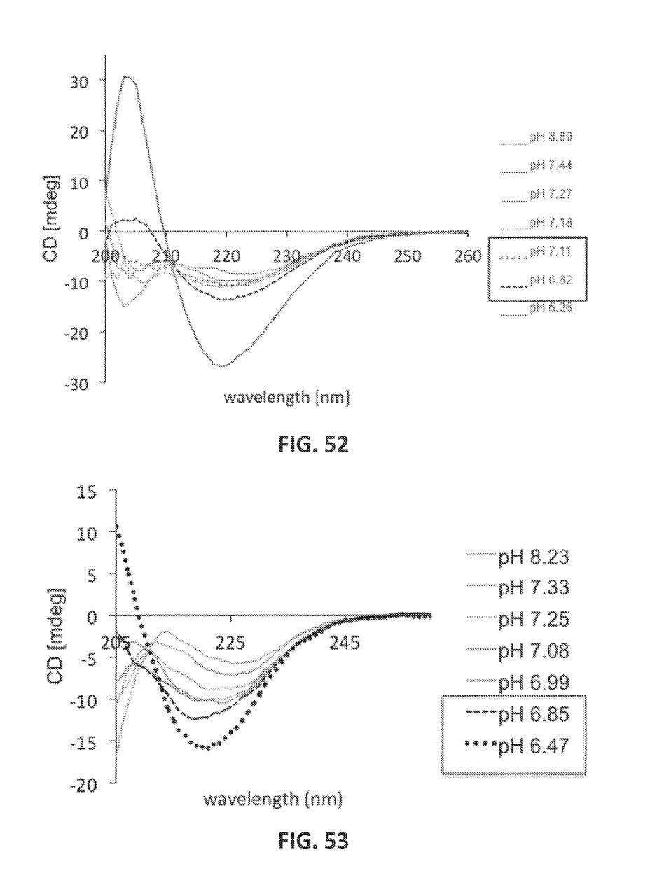

This Example shows that a vehicle that transitions at a pH of 6.6-7.4 in artificial serum (150 mM NaCl, 2.2 mM CaCl₂, and 1.8 mM of 20 kDa PEG) that also displays 2000 Da PEG on the surface can be constructed by mixing a PA that has a 2000 Da PEG termination on the lysine C₁₆-MAAAEEEEK(PEG₂₀₀₀)-NH₂ (PA79) (SEQ ID NO:40, bolded portion, along with a PA that has a DO3A:Gd imaging moiety (for example PA5). Addition of 2000 Da PEG to the lysine ε-amine significantly destabilizes nanofiber formation, making the transition pH shift back to a more acidic pH. For example, pure C₁₆-IAAAEEEEK(DO3A:Gd)—NH₂ (PA5) (SEQ ID NO:5, bolded portion) remains nanofibers across all measured pH values in both pure and artificial mouse serum (150 mM NaCl, 2.2 mM CaCl₂, and 1.8 mM of 20 kDa PEG). By making 100 M PA that is by molar concentration 10-15% PA79 and 80-85% PA5 makes this micelle to nanofiber transition occur at pH values between 6.6-7.4 in artificial mouse serum (FIG. 51-53). FIG. 51 shows the pH-dependent CD data of a 100 μM total PA concentration that is a mixture of 10% PA79 and 85% PA5 in a 150 mM NaCl, 2.2 mM CaCl₂, and 1.8 mM of 20 kDa PEG salt solution. Here, the micelle-to-nanofiber transition pH occurs between 7.04-8.31. FIG. 52 shows the pH-dependent CD data of 100 μM total PA concentration that is a mixture of 13% PA79 and 87% PA5 in the same salt solution. Here, the micelle-to-nanofiber transition pH occurs between 6.82-7.11. Finally, FIG. 53 shows the CD data of 100 μM total PA concentration that is a mixture of 15% PA79 and 85% PA5 in the same solution. Here, the micelle-to-nanofiber transition pH occurs between pH 6.47-6.85.

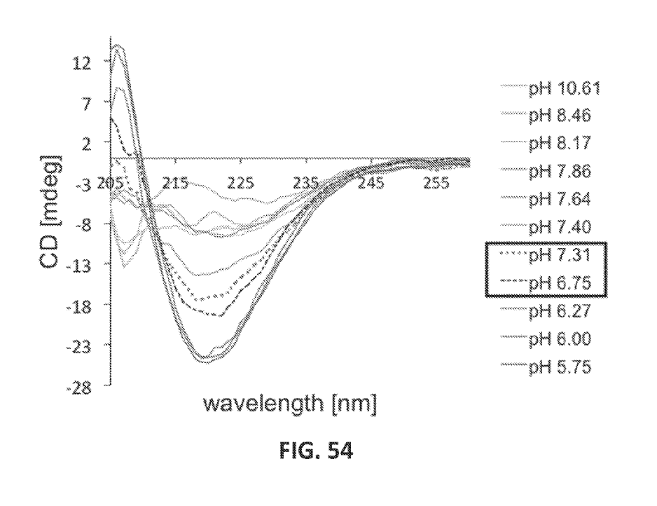

Finally, reducing the attractive forces of the PA by, for example, changing the peptide sequence, can enable the design of a micelle-to-nanofiber transition to occur in the same 6.6-7.4 pH range using a smaller percentage of PEG₂₀₀₀ terminated PA in the mixture. For instance, upon replacing PA5 with PA6, simply by substituting the Isoleucine residue with a Valine residue, which has slightly less β-sheet propensity, the micelle-to-nanofiber can be induced to occur at a pH of 6.6-7.4 with only 5% PA79. FIG. 54 shows the pH-dependent CD data of a 100 μM total PA concentration that is a mixture of 5% PA79 and 95% PA6 in a 150 mM NaCl, 2.2 mM CaCl₂, and 1.8 mM of 20 kDa PEG salt solution. Here, the micelle-to-nanofiber transition pH occurs between pH 6.75-7.31. Conversely, increasing the attractive forces of the PA molecule will result in a greater % of PEG₂₀₀₀ terminated PA to be part of the mixture to enable a transition to occur between pH of 6.6-7.4

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A composition comprising a plurality of biocompatible self-assembling molecules conjugated to a diagnostic or therapeutic agent,
wherein the plurality of peptide amphiphiles form spherical micelles when in a physiological environment having a pH of 7.30 to 7.45,
wherein the spherical micelles transform into cylindrical nanofibers when in a physiological environment having a pH less than 7.3, and
wherein the biocompatible self-assembling molecules are defined by Formula I
Cₙ—Z-A-X (I),
wherein:
Cₙ represents an alkyl, alkenyl, or alkynyl group;
Z represents a conjugate comprising Bⁱₒ, Uₚ, Neg_{q}, and optionally Y arranged any order, with the proviso that Bⁱₒ is positioned between Neg_{q} and Cₙ;
wherein Bⁱ, individually for each occurrence, represents an amino acid with intermediate beta-sheet propensity and o represents an integer from 1 to 2,
U, individually for each occurrence, represents an uncharged amino acid with poor beta-sheet propensity, and p represents an integer from 0 to 20,
Neg, individually for each occurrence, represents an anionic amino acid, and wherein q represents an integer from 3 to 7, and
Y is absent, or represents a spacer group comprising a diagnostic or therapeutic agent;
A is absent, or represents a hydrophilic linking group; and
X represents a terminating residue,
with the proviso that at least one of Y or A is present in the molecules defined by Formula I.

2. (canceled)

3. (canceled)

4. The composition of claim 1, wherein the spherical micelles have a hydrodynamic diameter of from about 8 nm to about 25 nm.

5. The composition of claim 1, wherein the cylindrical nanofibers are greater than about 200 nm in length.

6. The composition of claim 1, wherein the length of the cylindrical nanofibers are at least 10 times greater than the diameter of the cylindrical nanofibers.

7. The composition of claim 1, wherein the spherical micelles transform into cylindrical nanofibers when in a physiological environment having a pH between 5.1 to 7.3, or 6.4 to 7.3.

8. (canceled)

9. (canceled)

10. The composition of claim 1, wherein Y is present and wherein the diagnostic or therapeutic agent comprises a radionuclide, a paramagnetic metal ion, or a combination thereof.

11. The composition of claim 10, wherein the diagnostic or therapeutic agent comprises a chelating agent.

12. (canceled)

13. (canceled)

14. (canceled)

15. The composition of claim 11, wherein the chelating agent is complexed with Gd³⁺, Lu³⁺, Tb³⁺, Ga³⁺, or In³⁺.

16. The composition of claim 15, wherein Y comprises an amino acid conjugated to a metal chelator.

17. (canceled)

18. The composition of claim 1, wherein A comprises a hydrophilic oligo- or polyalkylene oxide.

19. (canceled)

20. (canceled)

21. (canceled)

22. (canceled)

23. (canceled)

24. The composition of claim 1, wherein the biocompatible self-assembling molecules comprise biocompatible self-assembling molecules defined by one of Formulae IIA-VB
Cₙ—Bⁱₒ—Uₚ-Neg_{q}-Y-A-X (IIA)
Cₙ—Bⁱₒ—Uₚ-Neg_{q}-Y—X (IIB)
Cₙ—Bⁱₒ—Uₚ—Y-Neg_{q}-A-X (IIIA)
Cₙ—Bⁱₒ—Uₚ—Y-Neg_{q}-X (IIIB)
Cₙ—Uₚ—Bⁱₒ—Y-Neg_{q}-A-X (IVA),
Cₙ—Uₚ—Bⁱₒ—Y-Neg_{q}-X (IVB),
Cₙ—Uₚ—Bⁱₒ-Neg_{q}-Y-A-X (VA), or
Cₙ—Uₚ-Bio-Neg_{q}-Y—X (VB),
wherein
Cₙ represents an alkyl, alkenyl, or alkynyl group;
Bⁱ, individually for each occurrence, represents an amino acid with high beta-sheet propensity and o represents an integer from 1 to 2;
U, individually for each occurrence, represents an uncharged amino acid with poor beta-sheet propensity, and p represents an integer from 0 to 20;
Neg, individually for each occurrence, represents an anionic amino acid, and wherein q represents an integer from 2 to 7;
Y represents spacer group comprising a diagnostic or therapeutic agent;
A when present, represents a hydrophilic linking group; and
X represents a terminating residue.

25. The composition of claim 1, wherein the biocompatible self-assembling molecules comprise biocompatible self-assembling molecules defined by one of Formulae IIC or IVC
Cₙ—Bⁱₒ—Uₚ-Neg_{q}-A-X (IIC), or
Cₙ—Uₚ—Bⁱₒ-Neg_{q}-A-X (IVC),
wherein
Cₙ represents an alkyl, alkenyl, or alkynyl group;
Bⁱ, individually for each occurrence, represents an amino acid with high beta-sheet propensity and o represents an integer from 1 to 2;
U, individually for each occurrence, represents an uncharged amino acid with poor beta-sheet propensity, and p represents an integer from 0 to 20;
Neg, individually for each occurrence, represents an anionic amino acid, and wherein q represents an integer from 2 to 7;
A represents a hydrophilic linking group; and
X represents a terminating residue.

26. (canceled)

27. (canceled)

28. The composition of claim 1, wherein the biocompatible self-assembling molecules comprise biocompatible self-assembling molecules defined by Formulae VI or VII:
(Cₙ)—(BⁱₒUₚ)-(Neg_{q}Y)-(—O—CH₂—CH₂-)ᵣ-propionic amide (VI), or
(Cₙ)—(BⁱₒUₚ)-(Neg_{q}Y)—NH₂ (VII),
wherein
Cₙ represents an alkyl, alkenyl, or alkynyl group;
Bⁱ, individually for each occurrence, represents an amino acid with high beta-sheet propensity and o represents an integer from 1 to 2;
U, individually for each occurrence, represents an uncharged amino acid with poor beta-sheet propensity, and p represents an integer from 0 to 20;
Neg, individually for each occurrence, represents an anionic amino acid, and wherein q represents an integer from 2 to 7;
r, when present, represents an integer ranging from 0 to 8; and
Y represents a lysine conjugated to a DO3A chelating agent optionally complexed with a trivalent metal ion.

29. The composition of claim 1, wherein the biocompatible self-assembling molecules comprise biocompatible self-assembling molecules defined by Formulae VIII or IX:
(Cₙ)—(BⁱₒUₚ)-(Neg_{q-1}Y)-(—O—CH₂—CH₂-)ᵣ-propionic acid (VIII), or
(Cₙ)—(BⁱₒUₚ)-(Neg_{q-1}Y)—COOH (IX),
wherein
Cₙ represents an alkyl, alkenyl, or alkynyl group;
Bⁱ, individually for each occurrence, represents an amino acid with intermediate beta-sheet propensity and o represents an integer from 1 to 2;
U, individually for each occurrence, represents an uncharged amino acid with poor beta-sheet propensity, and p represents an integer from 0 to 20;
Neg, individually for each occurrence, represents an anionic amino acid, and wherein q represents an integer from 2 to 7;
r, when present, represents an integer ranging from 0 to 8; and
Y represents a lysine conjugated to a DO3A chelating agent optionally complexed with a trivalent metal ion.

30. The composition of claim 28, wherein the ratio of n:o:q is 16-17:1:2-3 or 15-16:2:4-6, wherein n is an integer representing the number of carbon atoms in Cₙ.

31. The composition of claim 1, wherein the biocompatible self-assembling molecules comprise biocompatible self-assembling molecules defined by Formulae X:
(Cₙ)—(BⁱₒUₚ)-(Neg_{q})-(—O—CH₂—CH₂-)ᵣ-propionic amide (X)
wherein
Cₙ represents an alkyl, alkenyl, or alkynyl group;
Bⁱ, individually for each occurrence, represents an amino acid with high beta-sheet propensity and o represents an integer from 1 to 2;
U, individually for each occurrence, represents an uncharged amino acid with poor beta-sheet propensity, and p represents an integer from 0 to 20;
Neg, individually for each occurrence, represents an anionic amino acid, and wherein q represents an integer from 0 to 8; and
r, when present, represents an integer ranging from 1 to 150.

32. (canceled)

33. A composition comprising a plurality of biocompatible self-assembling molecules conjugated to a diagnostic or therapeutic agent,
wherein the plurality of peptide amphiphiles form spherical micelles when in a physiological environment having a pH of 7.30 to 7.45,
wherein the spherical micelles transform into cylindrical nanofibers when in a physiological environment having a pH less than 7.3, and
wherein the plurality of biocompatible self-assembling molecules comprises a mixture comprising a plurality of first biocompatible self-assembling molecules and a plurality of second biocompatible self-assembling molecules,
wherein the plurality of first biocompatible self-assembling molecules are defined by Formula XI
Cₙ-E-A-X (XI),
wherein:
Cₙ represents an alkyl, alkenyl, or alkynyl group;
E represents a conjugate comprising Bₒ, Uₚ, Neg_{q}, and Y arranged any order, with the proviso that Bₒ is positioned between Neg_{q} and Cₙ;
wherein B, individually for each occurrence, represents an amino acid with beta-sheet propensity and o represents an integer from 1 to 2, U, individually for each occurrence, represents an uncharged amino acid with poor beta-sheet propensity, and p represents an integer from 0 to 20, Neg, individually for each occurrence, represents an anionic amino acid, and wherein q represents an integer from 3 to 7, and
Y represents a spacer group comprising a diagnostic or therapeutic agent; A is absent, or represents a hydrophilic linking group; and
X represents a terminating residue; and
wherein the plurality of second biocompatible self-assembling molecules are defined by Formula XII
Cₙ—F-A-X (XII),
wherein:
Cₙ represents an alkyl, alkenyl, or alkynyl group;
F represents a conjugate comprising Bₒ, Uₚ, Neg_{q}, and optionally Y arranged any order, with the proviso that Bₒ is positioned between Neg_{q} and Cₙ;
wherein B, individually for each occurrence, represents an amino acid with beta-sheet propensity and o represents an integer from 1 to 2,
U, individually for each occurrence, represents an uncharged amino acid with poor beta-sheet propensity, and p represents an integer from 0 to 20,
Neg, individually for each occurrence, represents an anionic amino acid, and wherein q represents an integer from 3 to 7, and
Y is absent, or represents a spacer group comprising a diagnostic or therapeutic agent;
A represents a hydrophilic linking group; and
X represents a terminating residue.

34-66. (canceled)

67. A method for diagnosing cancer in a subject, comprising
(a) administering to the subject an effective amount of the composition of claim 1, wherein the biocompatible self-assembling molecules are conjugated to a diagnostic agent, and
(b) imaging the subject for the presence of the diagnostic agent,
wherein detection of an accumulated amount of the diagnostic agent in the subject is an indication of the presence of a tumor.

68. (canceled)

69. (canceled)

70. A method for treating cancer in a subject, comprising administering to the subject the composition of claim 1, wherein the biocompatible self-assembling molecules are conjugated to a therapeutic agent, and wherein therapeutic agent accumulates in the cancer of the subject in a therapeutically effective amount.

71. (canceled)

72. (canceled)

73. The composition of claim 1, consisting essentially of Palmitoyl-MAAAEEEEK(DO3A:Gd)—NH₂ (SEQ ID NO:40, underlined portion).
